SEQUENCE LISTING (141 sequences)

SEQ ID NO: 1 - protein, 115 aa, synthetic construct
QVQLQESGGGLVQAGGSLRLSCVASGSLFRWTAMAWYRQAPGKQRELVAVISPAGSIDYADSVKGRFTISRDNGKNVVYLQMNSLKPEDTAVYYCSIHHSARAYWGQGTQVTVSS

SEQ ID NO: 2 - protein, 111 aa, synthetic construct
QVQLQESGGGLVQPGGSLTLSCAASIHLFSRTAMDWYRQAPGRQRELVATISPVGSTYYADSAKDRFTISRDNANNMVYLQMNSLGPEDTAVYYCISGQLQGQGTQVTVSS

SEQ ID NO: 3 - protein, 119 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSS

SEQ ID NO: 4 - protein, 119 aa, synthetic construct
QVQLQQFGGGLVQAGGSLRLSCTASGSTFSFSNYHMGWYRQAPGKQRERVASISSRGSTYYSDSVKGRFAISRDTARNTVYLQMNSLEPEETAVYYCNARGRATGRDYWGQGTLVTVSS

SEQ ID NO: 5 - protein, 122 aa, synthetic construct
QVQLQQFGGGLVQPGGSLRLSCAVSRSIFSIDAMAWYRQAPGKQRNLVGTITSDGTTNYVDSAKGRFTISRDNAKNTVYLQMNSLKPEDTAVYFCNAGAVSRTGGYRPSGYWGQGTLVTVSS

SEQ ID NO: 6 - protein, 119 aa, synthetic construct
QVQLQESGGGLVQAGGSLRLSCTASGAYFIMGWFRQAPGKQRELVARTIRDGTTDYADSVKGRFTISRNTAENTAYLQMNSLKPEDTGVYYCAAGPLSKSFAPWDPAYWGQGTLVTVSS

SEQ ID NO: 7 - protein, 117 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSS

SEQ ID NO: 8 - protein, 123 aa, synthetic construct
QVQLQQFGGGLVQPGGSLTLSCAASGSIFSINPMGWYRQAPGKRRELVASLTNRGITTYADSVKGRFTISRDNAKNTVYLQMNALKPEDTAVYYCHAYVDTGSHEYPQYENYWGQGTLVTVSS

SEQ ID NO: 9 - protein, 111 aa, synthetic construct
QVQLQESGGGLVQAGGSLRLSCAASGSTFTFSTMAWYRQAPGKQRELVATFFSPSTWYADSVEGRFTVSRNNAKNTMYLHMNSLKPEDTAVYYCTSPSASNGQGTQVTVSS

SEQ ID NO: 10 - protein, 123 aa, synthetic construct
QVQLQESGGGLVQPGGSLTLSCAASGSIVSINPMGWYRQAPGKRRELVASLTNRGITTYADSVKGRFTISRDNAKNTVYLQMNTLKPEDTAVYYCHAYVDTGNYEYPQYENYWGQGTLVTVSS

SEQ ID NO: 11 - protein, 369 aa, synthetic construct
QVQLQESGGGLVQAGGSLRLSCVASGSLFRWTAMAWYRQAPGKQRELVAVISPAGSIDYADSVKGRFTISRDNGKNVVYLQMNSLKPEDTAVYYCSIHHSARAYWGQGTQVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 365 aa, synthetic construct
QVQLQESGGGLVQPGGSLTLSCAASIHLFSRTAMDWYRQAPGRQRELVATISPVGSTYYADSAKDRFTISRDNANNMVYLQMNSLGPEDTAVYYCISGQLQGQGTQVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 373 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 367 aa, synthetic construct
QVQLQQFGGGLVQPGGSLRLSCAVSRSIFSIDAMAWYRQAPGKQRNLVGTITSDGTTNYVDSAKGRFTISRDNAKNTVYLQMNSLKPEDTAVYFCNAGAVSRTGGYRPSGYWGQGTLVTVSSTSGSGGGGSGGGGSGGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 373 aa, synthetic construct
QVQLQESGGGLVQAGGSLRLSCTASGAYFIMGWFRQAPGKQRELVARTIRDGTTDYADSVKGRFTISRNTAENTAYLQMNSLKPEDTGVYYCAAGPLSKSFAPWDPAYWGQGTLVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 16 - protein, 371 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 17 - protein, 377 aa, synthetic construct
QVQLQQFGGGLVQPGGSLTLSCAASGSIFSINPMGWYRQAPGKRRELVASLTNRGITTYADSVKGRFTISRDNAKNTVYLQMNALKPEDTAVYYCHAYVDTGSHEYPQYENYWGQGTLVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 18 - protein, 365 aa, synthetic construct
QVQLQESGGGLVQAGGSLRLSCAASGSTFTFSTMAWYRQAPGKQRELVATFFSPSTWYADSVEGRFTVSRNNAKNTMYLHMNSLKPEDTAVYYCTSPSASNGQGTQVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 19 - protein, 377 aa, synthetic construct
QVQLQESGGGLVQPGGSLTLSCAASGSIVSINPMGWYRQAPGKRRELVASLTNRGITTYADSVKGRFTISRDNAKNTVYLQMNTLKPEDTAVYYCHAYVDTGNYEYPQYENYWGQGTLVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 20 - protein, 117 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 21 - protein, 108 aa, synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 22 - protein, 119 aa, synthetic construct
QVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSS

SEQ ID NO: 23 - protein, 111 aa, synthetic construct
EIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIK

SEQ ID NO: 24 - protein, 593 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRP

SEQ ID NO: 25 - protein, 215 aa, synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 144 aa, synthetic construct
MQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFQGRRASQ

SEQ ID NO: 27 - protein, 590 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSGGSGGGGSGGGGSGGGGSGGGGSGGEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 29 - protein, 218 aa, synthetic construct
EIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 590 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSGGSGGGGSGGGGSGGGGSGGGGSGGQVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 359 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSGGSGGGGSGGGGSGGGGSGGGGSGGEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 446 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 360 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSGGSGGGGSGGGGSGGGGSGGGGSGGEIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 448 aa, synthetic construct
QVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 357 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSGGSGGGGSGGGGSGGGGSGGGGSGGEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 362 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSGGSGGGGSGGGGSGGGGSGGGGSGGEIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 592 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSGGSGGGGSGGGGSGGGGSGGGGSGGQVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 588 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSGGSGGGGSGGGGSGGGGSGGGGSGGEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 360 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 40 - protein, 358 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 41 - protein, 224 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 573 aa, synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGQVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSS

SEQ ID NO: 43 - protein, 218 aa, synthetic construct
EIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 580 aa, synthetic construct
QVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSGGGGSGGGGSGGQVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSS

SEQ ID NO: 46 - protein, 571 aa, synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGQVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSS

SEQ ID NO: 47 - protein, 582 aa, synthetic construct
QVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSGGGGSGGGGSGGQVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSS

SEQ ID NO: 48 - protein, 359 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSGGSGGGGSGGGGSGGGGSGGGGSGGEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 368 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSGGSGGGGSGGGGSGGGGSGGGGSGGQVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 456 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSC

SEQ ID NO: 51 - protein, 442 aa, synthetic construct
EIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 52 - protein, 477 aa, synthetic construct
EMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIVNSSGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGSGGLNDIFEAQKIEWHE

SEQ ID NO: 53 - protein, 489 aa, synthetic construct
SQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFSYYVHYWEKGGIQQVKGPFRSNSISLDNLKPSRVYCLQVQAQLLWNKSNIFRVGHLSNISCYETMADASTELQQGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGSGGGSAWSHPQFEKGGGSGGGSGGSAWSHPQFEK

SEQ ID NO: 54 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 55 - protein, 244 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 56 - protein, 242 aa, synthetic construct
GLNDIFEAQKIEWHEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 244 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKSGGLNDIFEAQKIEWHE

SEQ ID NO: 58 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 59 - protein, 373 aa, synthetic construct
QVQLQQFGGGLVQAGGSLRLSCTASGSTFSFSNYHMGWYRQAPGKQRERVASISSRGSTYYSDSVKGRFAISRDTARNTVYLQMNSLEPEETAVYYCNARGRATGRDYWGQGTLVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 117 aa, synthetic construct
QVQLAESGGGLVQPGGSLTLSCVASGSILEMSRMAWYRQAPGKMRELVASITSIGSIVYADSVKGRFTLSRDNTKNTVYLQMNSLKPEDTAVYRCNALGTWSDETYWGQGTQVTVSS

SEQ ID NO: 61 - protein, 117 aa, synthetic construct
QVQLQQFGGGLVQPGGSLTLSCVASESISEMSRMAWYRQAPGKQRELVASITMHGGVVYADAVKGRFTISRDNTKNTVYLQMNSLKPEDTAVYRCNALGTWSDETYWGQGTLVTVSS

SEQ ID NO: 62 - protein, 117 aa, synthetic construct
QVQLQESGGGLVQPGGSLTLSCVASGSILEMSRMAWYRQAPGKQRELVASITSIGSKVYADSVKGRFTLSRDNTKNTIYLQMNSLKPEDTAVYRCNLLGTWSDETYWGQGTLVTVSS

SEQ ID NO: 63 - protein, 121 aa, synthetic construct
QVQLQASGGGLVQSGGSLRLSCVASGSTFSINGMGWYRQAPGKERELVATLTAGGNADYAVSVAGRFIISRGDKKNTRILQMNDLKPEDTAVYYCMADIYTGDSYSGVDYWGKGTLVTVSS

SEQ ID NO: 64 - protein, 117 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSS

SEQ ID NO: 65 - protein, 115 aa, synthetic construct
QVQLQQFGGGSVQAGGSLRLSCSAPGIIFEDIVMGWYRQGPGKQRELVALINSAVTDYADSVKGRFTISRDNAKNLVYLQMNSLRSEDTAVYYCTAIDTNWAQYWGQGTQVTVSS

SEQ ID NO: 66 - protein, 117 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSS

SEQ ID NO: 67 - protein, 120 aa, synthetic construct
QVQLQESGGGLVQPGGSLRLSCAASGSIFSGNAMGWYRQAPGKQRELVASITSGGDTHYVDSVKGRFTISRDNAKNMVYLQMSSLKPEDTAVYYCNAQERVYSDYAFASWGPGTQVTVSS

SEQ ID NO: 68 - protein, 115 aa, synthetic construct
QVQLQESGGGLVQPGGSLRLSCAASGFTFSTYVMNWVRQAPGKGLEWVSGIDSDGDSTTYTDSVKGRFTISRDNAKNTLYLQMNSLKPEDTAVYYCSMFRMATTGSQGTQVTVSS

SEQ ID NO: 69 - protein, 119 aa, synthetic construct
QVQLQESGGGLVQAGGSLRLSCAASGITSSIYAMGWYRQAPGNEREPVALITSGDNTNYPDSVKGRFTISRDTAKNTVYLQMSSLKPEDTAVYYCYADVVIGTTYYTSWGQGTLVTVSS

SEQ ID NO: 70 - protein, 371 aa, synthetic construct
QVQLAESGGGLVQPGGSLTLSCVASGSILEMSRMAWYRQAPGKMRELVASITSIGSIVYADSVKGRFTLSRDNTKNTVYLQMNSLKPEDTAVYRCNALGTWSDETYWGQGTQVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 371 aa, synthetic construct
QVQLQQFGGGLVQPGGSLTLSCVASESISEMSRMAWYRQAPGKQRELVASITMHGGVVYADAVKGRFTISRDNTKNTVYLQMNSLKPEDTAVYRCNALGTWSDETYWGQGTLVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 371 aa, synthetic construct
QVQLQESGGGLVQPGGSLTLSCVASGSILEMSRMAWYRQAPGKQRELVASITSIGSKVYADSVKGRFTLSRDNTKNTIYLQMNSLKPEDTAVYRCNLLGTWSDETYWGQGTLVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 371 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 371 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 75 - protein, 374 aa, synthetic construct
QVQLQESGGGLVQPGGSLRLSCAASGSIFSGNAMGWYRQAPGKQRELVASITSGGDTHYVDSVKGRFTISRDNAKNMVYLQMSSLKPEDTAVYYCNAQERVYSDYAFASWGPGTQVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 76 - protein, 369 aa, synthetic construct
QVQLQESGGGLVQPGGSLRLSCAASGFTFSTYVMNWVRQAPGKGLEWVSGIDSDGDSTTYTDSVKGRFTISRDNAKNTLYLQMNSLKPEDTAVYYCSMFRMATTGSQGTQVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 77 - protein, 373 aa, synthetic construct
QVQLQESGGGLVQAGGSLRLSCAASGITSSIYAMGWYRQAPGNEREPVALITSGDNTNYPDSVKGRFTISRDTAKNTVYLQMSSLKPEDTAVYYCYADVVIGTTYYTSWGQGTLVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 78 - protein, 366 aa, synthetic construct
QVQLQASGGGLVQSGGSLRLSCVASGSTFSINGMGWYRQAPGKERELVATLTAGGNADYAVSVAGRFIISRGDKKNTRILQMNDLKPEDTAVYYCMADIYTGDSYSGVDYWGKGTLVTVSSTSGSGGGGSGGGGSGGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 79 - protein, 369 aa, synthetic construct
QVQLQQFGGGSVQAGGSLRLSCSAPGIIFEDIVMGWYRQGPGKQRELVALINSAVTDYADSVKGRFTISRDNAKNLVYLQMNSLRSEDTAVYYCTAIDTNWAQYWGQGTQVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 80 - protein, 120 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSS

SEQ ID NO: 81 - protein, 111 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIK

SEQ ID NO: 82 - protein, 124 aa, synthetic construct
EVQLLESGGGLVQPGGSLRLSCAASGFTFSSYAMSWVRQAPGKGLEWVSAITGSGGSTYYADSVKGRFTISRDNSRNTLYLQMNSLRAEDTAVYYCAKGEGYAGSSYFRASDIWGQGTMVTVSS

SEQ ID NO: 83 - protein, 107 aa, synthetic construct
DIQMTQSPSSLSASVGDRVTITCRASQSISSYLNWYQQKPGKAPKLLIYTASSLQSGVPSRFSGSGSGTDFTLTISSLQPEDFATYYCQQSYSTPLTFGGGTKVEIK

SEQ ID NO: 84 - protein, 461 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGAQDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 85 - protein, 470 aa, synthetic construct
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAGAQDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 86 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 87 - protein, 218 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 89 - protein, 450 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 90 - protein, 594 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSTSGSGGGGSGGGGSGGGGSGGGGSGGGGEVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 91 - protein, 359 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSTSGSGGGGSGGGGSGGGGSGGGGSGGGGDIQMTQSPSSLSASVGDRVTITCRASQSISSYLNWYQQKPGKAPKLLIYTASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 453 aa, synthetic construct
EVQLLESGGGLVQPGGSLRLSCAASGFTFSSYAMSWVRQAPGKGLEWVSAITGSGGSTYYADSVKGRFTISRDNSRNTLYLQMNSLRAEDTAVYYCAKGEGYAGSSYFRASDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 93 - protein, 214 aa, synthetic construct
DIQMTQSPSSLSASVGDRVTITCRASQSISSYLNWYQQKPGKAPKLLIYTASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 598 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSTSGSGGGGSGGGGSGGGGSGGGGSGGGGEVQLLESGGGLVQPGGSLRLSCAASGFTFSSYAMSWVRQAPGKGLEWVSAITGSGGSTYYADSVKGRFTISRDNSRNTLYLQMNSLRAEDTAVYYCAKGEGYAGSSYFRASDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 95 - protein, 363 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSTSGSGGGGSGGGGSGGGGSGGGGSGGGGDIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 449 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 97 - protein, 345 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSGGSGGQVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 98 - protein, 338 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSGGSGGEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 99 - protein, 454 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGEIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 450 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNAYTQKSLSLSPG

SEQ ID NO: 101 - protein, 340 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSGGSGGEIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 348 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSGGSGGEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 459 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGQVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 104 - protein, 439 aa, synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNAYTQKSLSLSPG

SEQ ID NO: 105 - protein, 337 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSGGSGGEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 341 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSGGSGGEIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 107 - protein, 457 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 108 - protein, 452 aa, synthetic construct
QVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNAYTQKSLSLSPG

SEQ ID NO: 109 - protein, 367 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSGGSGGGGSGGGGSGGGGSGGGGSGGQVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 110 - protein, 356 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSGGSGGGGSGGGGSGGGGSGGGGSGGEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 111 - protein, 339 aa, synthetic construct
QVQLQESGGGLVQAGGSLRLSCTASGAYFIMGWFRQAPGKQRELVARTIRDGTTDYADSVKGRFTISRNTAENTAYLQMNSLKPEDTGVYYCAAGPLSKSFAPWDPAYWGQGTLVTVSSGGSGGEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 353 aa, synthetic construct
QVQLQQFGGGLVQPGGSLRLSCAVSRSIFSIDAMAWYRQAPGKQRNLVGTITSDGTTNYVDSAKGRFTISRDNAKNTVYLQMNSLKPEDTAVYFCNAGAVSRTGGYRPSGYWGQGTLVTVSSGGSGGQVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 442 aa, synthetic construct
EIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNAYTQKSLSLSPG

SEQ ID NO: 114 - protein, 360 aa, synthetic construct
QVQLQQFGGGLVQPGGSLRLSCAVSRSIFSIDAMAWYRQAPGKQRNLVGTITSDGTTNYVDSAKGRFTISRDNAKNTVYLQMNSLKPEDTAVYFCNAGAVSRTGGYRPSGYWGQGTLVTVSSGGSGGGGSGGGGSGGQVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 115 - protein, 348 aa, synthetic construct
QVQLQESGGGLVQAGGSLRLSCTASGAYFIMGWFRQAPGKQRELVARTIRDGTTDYADSVKGRFTISRNTAENTAYLQMNSLKPEDTGVYYCAAGPLSKSFAPWDPAYWGQGTLVTVSSGGSGGGGSGGGGSGGEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 116 - protein, 342 aa, synthetic construct
QVQLQQFGGGLVQPGGSLRLSCAVSRSIFSIDAMAWYRQAPGKQRNLVGTITSDGTTNYVDSAKGRFTISRDNAKNTVYLQMNSLKPEDTAVYFCNAGAVSRTGGYRPSGYWGQGTLVTVSSGGSGGEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 341 aa, synthetic construct
QVQLQESGGGLVQAGGSLRLSCTASGAYFIMGWFRQAPGKQRELVARTIRDGTTDYADSVKGRFTISRNTAENTAYLQMNSLKPEDTGVYYCAAGPLSKSFAPWDPAYWGQGTLVTVSSGGSGGEIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 118 - protein, 5 aa, synthetic construct
GGGGS

SEQ ID NO: 119 - protein, 10 aa, synthetic construct
GGGGSGGGGS

SEQ ID NO: 120 - protein, 15 aa, synthetic construct
GGGGSGGGGSGGGGS

SEQ ID NO: 121 - protein, 25 aa, synthetic construct
GGGGSGGGGSGGGGSGGGGSGGGGS

SEQ ID NO: 122 - protein, 360 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGDIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 459 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGEVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSC

SEQ ID NO: 124 - protein, 438 aa, synthetic construct
DIQMTQSPSSLSASVGDRVTITCRASQSISSYLNWYQQKPGKAPKLLIYTASSLQSGVPSRFSGSGSGTDFTLTISSLQPEDFATYYCQQSYSTPLTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 125 - protein, 373 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGEVQLLESGGGLVQPGGSLRLSCAASGFTFSSYAMSWVRQAPGKGLEWVSAITGSGGSTYYADSVKGRFTISRDNSRNTLYLQMNSLRAEDTAVYYCAKGEGYAGSSYFRASDIWGQGTMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 442 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 127 - protein, 369 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGEVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 463 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGEVQLLESGGGLVQPGGSLRLSCAASGFTFSSYAMSWVRQAPGKGLEWVSAITGSGGSTYYADSVKGRFTISRDNSRNTLYLQMNSLRAEDTAVYYCAKGEGYAGSSYFRASDIWGQGTMVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSC

SEQ ID NO: 129 - protein, 356 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGDIQMTQSPSSLSASVGDRVTITCRASQSISSYLNWYQQKPGKAPKLLIYTASSLQSGVPSRFSGSGSGTDFTLTISSLQPEDFATYYCQQSYSTPLTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 595 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGEVQLLESGGGLVQPGGSLRLSCAASGFTFSSYAMSWVRQAPGKGLEWVSAITGSGGSTYYADSVKGRFTISRDNSRNTLYLQMNSLRAEDTAVYYCAKGEGYAGSSYFRASDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 131 - protein, 356 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGDIQMTQSPSSLSASVGDRVTITCRASQSISSYLNWYQQKPGKAPKLLIYTASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 356 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGDIQMTQSPSSLSASVGDRVTITCRASQSISSYLNWYQQKPGKAPKLLIYTASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 356 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGDIQMTQSPSSLSASVGDRVTITCRASQGISSWLAWYQQKPGKAPKLLIYEASSLQSGVPSRFSGSGSGTDFTLTISSLQPEDFATYYCQQANQFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 446 aa, synthetic construct
EVQLLESGGGLVQPGGSLRLSCAASGFTFSSYWMSWVRQAPGKGLEWVSAISGSGGSRYYAESVKGRFTISRDNSKNTLYLQMNSLRAEDTAVYYCASSPLQWIDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 135 - protein, 356 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGDIQMTQSPSSLSASVGDRVTITCRASQGISSWLAWYQQKPGKAPKLLIYEASSLQSGVPSRFSGSGSGTDFTLTISSLQPEDFATYYCQQANQFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 595 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGEVQLLESGGGLVQPGGSLRLSCAASGFTFSSYAMSWVRQAPGKGLEWVSAITGSGGSTYYADSVKGRFTISRDNSRNTLYLQMNSLRAEDTAVYYCAKGEGYAGSSYFRASDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 137 - protein, 214 aa, synthetic construct
DIQMTQSPSSLSASVGDRVTITCRASQGISSWLAWYQQKPGKAPKLLIYEASSLQSGVPSRFSGSGSGTDFTLTISSLQPEDFATYYCQQANQFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 588 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGEVQLLESGGGLVQPGGSLRLSCAASGFTFSSYWMSWVRQAPGKGLEWVSAISGSGGSRYYAESVKGRFTISRDNSKNTLYLQMNSLRAEDTAVYYCASSPLQWIDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 139 - protein, 588 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGEVQLLESGGGLVQPGGSLRLSCAASGFTFSSYWMSWVRQAPGKGLEWVSAISGSGGSRYYAESVKGRFTISRDNSKNTLYLQMNSLRAEDTAVYYCASSPLQWIDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 140 - protein, 117 aa, synthetic construct
EVQLLESGGGLVQPGGSLRLSCAASGFTFSSYWMSWVRQAPGKGLEWVSAISGSGGSRYYAESVKGRFTISRDNSKNTLYLQMNSLRAEDTAVYYCASSPLQWIDVWGQGTTVTVSS

SEQ ID NO: 141 - protein, 107 aa, synthetic construct
DIQMTQSPSSLSASVGDRVTITCRASQGISSWLAWYQQKPGKAPKLLIYEASSLQSGVPSRFSGSGSGTDFTLTISSLQPEDFATYYCQQANQFPFTFGPGTKVDIK

(19) United States
(12) Patent Application Publication
(10) Pub. No.: US 2026/0085113 A1
(43) Pub. Date: Mar. 26, 2026

(54) ALL-IN-ONE AGONISTIC ANTIBODIES

(71) Applicant: Hoffmann-La Roche Inc., Little Falls, NJ (US)

(72) Inventors: Petra Elisabeth Marilena BALBI, Schlieren (CH); Jack Anthony BATES, Oxford (GB); Samuele CALABRO, Schlieren (CH); Laura CODARRI DEAK, Au (CH); Stephan GASSER, Fislisbach (CH); Guy GEORGES, Habach (DE); Xavier GUERIPEL, Zurich (CH); Ralf Joerg HOSSE, Mettmenstetten (CH); Christian KLEIN, Bonstetten (CH); Stéphane Gérard Alain LECLAIR, Gauting (DE); Ekkehard MOESSNER, Kreuzlingen (CH); Christian MUELLER, Wettswil (CH); Diana Angela PIPPIG, Muenchen (DE); Laurene POUSSE, Schlieren (CH); Camilla Elizabeth TREVOR, Bergdietikon (CH); Pablo UMAÑA FERNÁNDEZ, Lachen (CH)

(73) Assignee: Hoffmann-La Roche Inc., Little Falls, NJ (US)

(21) Appl. No.: 19/348,515

(22) Filed: Oct. 2, 2025

Related U.S. Application Data
(63) Continuation of application No. PCT/EP2024/058833, filed on Apr. 2, 2024.

(30) Foreign Application Priority Data
Apr. 3, 2023 (EP) 23166348.5
Apr. 3, 2023 (EP) 23166351.9

(51) Int. Cl. C07K 16/24; C07K 16/40

(52) U.S. Cl. CPC C07K 16/246; C07K 16/40; C07K 2317/31; C07K 2317/35; C07K 2317/522; C07K 2317/55; C07K 2317/569

(57) ABSTRACT

The application relates to antigen binding molecules comprising a pair of biparatopic target-binding domains, a pair of cytokine receptor-binding domains and an Fc domain, wherein the target-binding domains simultaneously bind the target antigen and the cytokine receptor-binding domains bind subunits of a cytokine receptor complex. Biparatopic assembly of the cytokine receptor-binding domains in presence of the target antigen allows to selectively activate cytokine receptors and effectively mimic cytokine activity in a targeted manner.

### CROSS-REFERENCE TO RELATED APPLICATIONS

This application is a continuation of International Application No. PCT/EP2024/058833 filed Apr. 2, 2024, which claims the benefit of and priority to European Patent Application No. 23166351.9, filed Apr. 3, 2023 and European Patent Application No. 23166348.5, filed Apr. 3, 2023, each of which is incorporated herein by reference in its entirety.

### REFERENCE TO AN ELECTRONIC SEQUENCE LISTING

The instant application contains a Sequence Listing submitted electronically in XML format and is hereby incorporated herein by reference in its entirety. Said XML copy, created on Sep. 15, 2025, is named P38388-US-1_SEQ_LISTING.xml, and is 167,797 bytes in size.

### FIELD OF THE INVENTION

The application relates to antigen binding molecules comprising a pair of biparatopic target-binding domains, a pair of cytokine receptor-binding domains and an Fc domain, wherein the target-binding domains simultaneously bind the target antigen and the cytokine receptor-binding domains bind subunits of a cytokine receptor complex. Biparatopic assembly of the cytokine receptor-binding domains in presence of the target antigen allows to selectively activate cytokine receptors and effectively mimic cytokine activity in a targeted manner.

### BACKGROUND OF THE INVENTION

In recent years, immunotherapy treatments for cancer have grown dramatically, and cancer immunotherapy is becoming a major strategy for combating disease. For many cancer types, immune checkpoint modulators, including anti-PD-1, have become the standard of care. However, despite all the advances made in the field of cancer immunotherapy treatment in recent years, a significant proportion of patients still fail to respond to available immunotherapies because of intrinsic or adaptive mechanisms of resistance. Cancer immunotherapy patients with non-inflamed immune conditions are more likely to not respond to immunotherapies. Immune cell infiltration in tumors has been shown to correlate with the ability of patients to respond to immunotherapy treatments. Developing new therapies aimed at increasing immune cell infiltration and enhancing immunogenicity is essential for patients.

In parallel with the above developments, cytokines have gained great interest as potential cancer treatments. IFNγ (interferon gamma or IFN-γ) is a cytokine that is mainly produced by activated lymphocytes, such as CD4⁺ and CD8⁺ T cells, and natural killer cells (NK cells) in response to immune or inflammatory stimuli. IFNγ is a homodimer and its receptors (IFNγR1 and IFNγR2) are expressed across hematopoietic and non-hematopoietic cells. On the cell surface, IFNγR1 is stably expressed whereas IFNγR2 is differentially expressed and regulates IFNγ. When IFNγ binds to its receptors, Janus kinases JAK1 and JAK2 are recruited and activated, which phosphorylate and activate STAT1. A phosphorylated STAT1 translocates to the nucleus, binds promoters, and modulates gene transcription under the control of IFNγ.

In contrast to other cancer therapies currently on the market or in development, IFNγ can act on both tumor cells and immune cells including T cells and dendritic cells. The effects of IFNγ on different cell types have several benefits, which include 1) enhanced expression of MHC-I molecules on the surface of both tumor cells and antigen-presenting cells, 2) the recruitment of immune cells to the tumor site through the induction of CXCL9, CXCL10 and CXCL11 production and 3) the ability to increase tumor antigen cross-presentation with a subsequent enhancement of an anti-tumor immune response. In addition to these effects, IFNγ promotes the formation of a Th1 environment, monocyte differentiation, macrophage polarization, and angiogenesis. As IFNγ receptors are expressed on a wide range of cell types, sink effects may diminish its activity.

IFNγ can also show undesirable side effects. In addition, problems with administration, bioavailabilty and short half-life may arise. Thus, there is a need for new molecules able to selectively activate the IFNγ pathway at the tumor site.

Targeting the Interleukin 2 (IL-2) pathway as a cancer immunotherapy strategy has a long and ramified history, serving as testament to both the triumphs and the complexities of this clinical approach. IL-2 is a cytokine that activates lymphocytes and natural killer (NK) cells. Efficacy in the clinic is often overshadowed by reports of IL-2-associated toxicity linked to peripheral T cell activity and CD25-mediated complications.

Cytokines are powerful immune modulators that initiate signaling through receptor dimerization, but natural cytokines have some limitations as therapeutics. Low stability and difficulties in the production process are just some of them. It is known for some time that antibodies can induce signaling on cells, and therefore substitute a natural ligand.

In the recent literature there is growing evidence that bringing together heterodimeric cytokine receptors using both antibody and non-antibody based protein scaffolds is a viable strategy to mimic the activity of native cytokines. For instance, Moraga and colleagues provide an early example of using diabodies as surrogate ligands for Erythropoietin receptor (EpoR) (Moraga et al. Cell 160, 1196-1208 (2015)). Researchers at Teneobio combined heavy chain-only antibodies (VHHs fused to an Fc domain) against different epitopes on the Interleukin-2 receptors (IL-2Rβ and IL-2Rγ) into a bispecific effectorless IgG₄ Fc (CHI deleted) using knob-in-hole technology. While monospecific anti-IL-2Rβ or anti-IL-2Rγ alone or in mixture did not induce STAT5 phosphorylation on human CD8⁺ T cells, bispecific anti-IL-2Rβγ antibodies showed varying levels of agonist activity (Harris, K. E., et al. Sci Rep 11 (1): 10592 (2021)). A similar approach as described by Teneobio, was undertaken by scientists at Synthekine, which described the functional induction of signaling of the two interleukin receptors (IL-2Rβ and IL-2Rγ) via single domain antibodies (sdAbs: WO 2022/032040 A1), and reviewed by Saxton and colleagues (Saxton, R. A., et al. Nat Rev Drug Discov. 22, 21-37 (2022)). This approach was further expanded by researchers at Stanford University where the authors presented a strategy to discovery cytokine surrogate agonists by using modular ligands like VHH or scFv human for interleukin-2/15, type-I interferon, and interleukin-10 receptors. Interestingly, they also identified functional, non-natural assemblies like the IL-2Rβ/IL-10Rβ heterodimer (Yen, M., et al. Cell 185 (8): 1414-1430 e1419 (2022)). The same authors also discuss a structure-based approach for the decoupling of the pro- and anti-inflammatory functions of interleukin-10 (Saxton, R. A., et al. Science 371 (6535) (2021)). Two academic research groups from Czech Republic and Israel reported together the discovery of non-antibody based scaffolds that mimic the cytokine IFNλ. Combinatorial libraries derived from several established small protein scaffolds were used to identify variants capable of binding to the IFNλR1 or IL-10Rβ and induce functional signaling (Kolarova, L., et al. FEBS J 289 (9): 2672-2684 (2022)). Reducing the size of the agonistic modules was addressed by researchers at Medikine. They obtained molecules selected from peptide libraries by screens designed to identify molecules binding simultaneously to the Rα and γc subunits of the human IL-7 receptor. Those peptides, with an molecular weight of less than 5 kDa fused to an IgG₁-Fc domain exhibits biological properties similar to those of IL-7 in vitro, and when administered to non-human primates (Dower, W., et al. Journal for Immuno Therapy of Cancer 8 (Suppl 3): A341-A342 (2020)).

Due to the pleiotropic effects of cytokines, there is a need for novel approaches to selectively activate cytokine receptors and effectively mimic cytokine activity.

### SUMMARY OF THE INVENTION

The present invention relates to novel antigen binding molecules comprising cytokine receptor-binding domains, which, under the desired conditions of biparatopic assembly on a target antigen, serve as cytokine mimetics selectively activating the receptor pathway.

The present invention relates to an antigen binding molecule comprising i) a first target-binding domain, ii) a second target-binding domain, iii) a first cytokine receptor-binding domain, iv) a second cytokine receptor-binding domain, and v) a Fc domain, wherein the first target-binding domain is capable of binding a first epitope on the target antigen and the second target-binding domain is capable of binding a second epitope on the target antigen, wherein the first and the second target-binding domains do not compete for binding on the tumor-associated antigen; and wherein the first cytokine receptor-binding domain is capable of binding a first cytokine receptor subunit and the second cytokine receptor-binding domain is capable of binding a second cytokine receptor subunit.

In one aspect, first and second target-binding domains are antibody fragments, particularly Fv, Fab, scFv, scFab molecules or single domain antibodies. In one aspect, the first and second target-binding domain are Fab molecules. In one aspect, the first target-binding domain comprises a heavy chain variable domain (VH₁), a light chain variable domain (VL₁), a heavy chain constant domain (CH1₁) and a light chain constant domain (CL₁) and the second target-binding domain comprises a heavy chain variable domain (VH₂), a light chain variable domain (VL₂), a heavy chain constant domain (CH1₂) and a light chain constant domain (CL₂). In one aspect, the first target-binding domain and/or the second target-binding domain is a cross-Fab molecule. In one aspect, the first target-binding domain and/or the second target-binding domain comprise charge mutations. In one aspect, the first target-binding domain is a cross-Fab and the second target-binding domain comprises charge mutations or wherein the second target-binding domain is a cross-Fab and the first target-binding domain comprises charge mutations.

In one aspect, the first target-binding domain and the second target-binding domain specifically bind to a tumor-associated antigen or a T cell antigen.

In one aspect, the first target-binding domain and the second target-binding domain specifically bind to FAP, PD-1, Her2, Her3, LAG-3, CEA or EGFR. In one aspect, a) the first target-binding domain comprises a VH₁ of SEQ ID NO: 20 and a VL₁ of SEQ ID NO: 21 and the second target-binding domain comprises a VH₂ of SEQ ID NO: 22 and a VL₂ of SEQ ID NO: 23, or b) the first target-binding domain comprises a VH₁ of SEQ ID NO: 22 and a VL₁ of SEQ ID NO: 23 and the second target-binding domain comprises a VH₂ of SEQ ID NO: 20 and a VL₂ of SEQ ID NO: 21, or c) the first target-binding domain comprises a VH₁ of SEQ ID NO: 80 and a VL₁ of SEQ ID NO: 81 and the second target-binding domain comprises a VH₂ of SEQ ID NO: 82 and a VL₂ of SEQ ID NO: 83, or d) the first target-binding domain comprises a VH₁ of SEQ ID NO: 82 and a VL₁ of SEQ ID NO: 83 and the second target-binding domain comprises a VH₂ of SEQ ID NO: 80 and a VL₂ of SEQ ID NO: 81, e) the first target-binding domain comprises a VH₁ of SEQ ID NO: 82 and a VL₁ of SEQ ID NO: 83 and the second target-binding domain comprises a VH₂ of SEQ ID NO: 140 and a VL₂ of SEQ ID NO: 141: or f) the first target-binding domain comprises a VH₁ of SEQ ID NO: 140 and a VL₁ of SEQ ID NO: 141 and the second target-binding domain comprises a VH₂ of SEQ ID NO: 82 and a VL₂ of SEQ ID NO: 83.

In one aspect, both the first and second cytokine receptor subunits are subunits of the IFNγ receptor complex or IL-2 receptor complex. In one aspect, a) the first cytokine receptor-binding domain is capable of binding IFNγR1 and the second cytokine receptor-binding domain is capable of binding IFNγR2, or b) the first cytokine receptor-binding domain is capable of binding IFNγR2 and the second cytokine receptor-binding domain is capable of binding IFNγR1: or c) the first cytokine receptor-binding domain is capable of binding IL-2Rβ and the second cytokine receptor-binding domain is capable of binding IL-2Rγ, or d) the first cytokine receptor-binding domain is capable of binding IL-2Rγ and the second cytokine receptor-binding domain is capable of binding IL-2Rβ.

In one aspect, the first and second cytokine receptor-binding domains are antibody fragments, particularly Fv, Fab, scFv, scFab, single domain antibodies or VHH domains. In one aspect, the first and second cytokine receptor-binding domains are VHH domains. In one aspect, a) the first cytokine receptor-binding domain comprises an amino acid sequence selected from SEQ ID NO: 1, SEQ ID NO: 2, SEQ ID NO: 3 and SEQ ID NO: 5, and the second cytokine receptor-binding domain comprises an amino acid sequence selected from SEQ ID NO: 6, SEQ ID NO: 7 and SEQ ID NO: 9, or b) the first cytokine receptor-binding domain comprises an amino acid sequence selected from SEQ ID NO: 6, SEQ ID NO: 7 and SEQ ID NO: 9, and the second cytokine receptor-binding domain comprises an amino acid sequence selected from SEQ ID NO: 1, SEQ ID NO: 2, SEQ ID NO: 3 and SEQ ID NO: 5: or c) the first cytokine receptor-binding domain comprises an amino acid sequence selected from SEQ ID NO: 60, SEQ ID NO: 61, SEQ ID NO: 62 and SEQ ID NO: 64, and the second cytokine receptor-binding domain comprises an amino acid sequence selected from SEQ ID NO: 66, SEQ ID NO: 67, SEQ ID NO: 68 and SEQ ID NO: 69, or d) the first cytokine receptor-binding domain comprises an amino acid sequence selected from SEQ ID NO: 66, SEQ ID NO: 67, SEQ ID NO: 68 and SEQ ID NO: 69, and the second cytokine receptor-binding domain comprises an amino acid sequence selected from SEQ ID NO: 60, SEQ ID NO: 61, SEQ ID NO: 62 and SEQ ID NO: 64.

In one aspect, the Fc domain comprises a first Fc domain subunit and a second Fc domain subunit. In one aspect, the Fc domain is an IgG, particularly an IgG₁, Fc domain. In one aspect, the Fc domain is a human Fc domain. 19. The antigen binding molecule according to any one of the preceding claims, where In one aspect, in the Fc domain comprises a modification promoting the association of the first and the second subunit of the Fc domain. In one aspect, the Fc domain comprises one or more amino acid substitution that reduces binding to an Fc receptor and/or effector function.

In one aspect, the first cytokine receptor-binding domain is fused at its C-terminus to the N-terminus of VH₁ or VL₁ of the first target-binding domain, and the first target-binding domain is fused at its N-terminus of VH₁ or VL₁ to the C-terminus of the first Fc domain subunit, and the second cytokine receptor-binding domain is fused at its C-terminus to the N-terminus of VH₂ or VL₂ of the second target-binding domain, and the second target-binding domain is fused at its C-terminus of CH1₂ or CL₁ to the N-terminus of the second Fc domain subunit. In one aspect, the antigen binding molecules comprise a) a first polypeptide comprising in order from the N-terminus to C-terminus a first Fc domain subunit, a VH₁ and a CH1₁, a second polypeptide comprising in order from the N-terminus to C-terminus a the first cytokine receptor-binding domain, a VL₁ and a CL₁, a third polypeptide comprising in order from the N-terminus to C-terminus a VL₂, a CH1₂ and a second Fc domain subunit, and a fourth polypeptide comprising in order from the N-terminus to C-terminus a the second cytokine receptor-binding domain, a VH₂ and a CL₂: or b) a first polypeptide comprising in order from the N-terminus to C-terminus a first Fc domain subunit, a VL₁ and a CH1₁, a second polypeptide comprising in order from the N-terminus to C-terminus a the first cytokine receptor-binding domain, a VH₁ and a CL₁, a third polypeptide comprising in order from the N-terminus to C-terminus a VH₂, a CH1₂ and a second Fc domain subunit, and a fourth polypeptide comprising in order from the N-terminus to C-terminus a the second cytokine receptor-binding domain, a VL₂ and a CL₂: or c) a first polypeptide comprising in order from the N-terminus to C-terminus a first Fc domain subunit, a VL₁ and a CL₁, a second polypeptide comprising in order from the N-terminus to C-terminus a the first cytokine receptor-binding domain, a VH₁ and a CH1₁, a third polypeptide comprising in order from the N-terminus to C-terminus a VL₂, a CH1₂ and a second Fc domain subunit, and a fourth polypeptide comprising in order from the N-terminus to C-terminus a the second cytokine receptor-binding domain, a VH₂ and a CL₂: or d) a first polypeptide comprising in order from the N-terminus to C-terminus a first Fc domain subunit, a VH₁ and a CL₁, a second polypeptide comprising in order from the N-terminus to C-terminus a the first cytokine receptor-binding domain, a VL₁ and a CH1₁, a third polypeptide comprising in order from the N-terminus to C-terminus a VH₂, a CH1₂ and a second Fc domain subunit, and a fourth polypeptide comprising in order from the N-terminus to C-terminus a the second cytokine receptor-binding domain, a VL₂ and a CL₂: or e) a first polypeptide comprising in order from the N-terminus to C-terminus a first Fc domain subunit, a VH₁ and a CH1₁, a second polypeptide comprising in order from the N-terminus to C-terminus a the first cytokine receptor-binding domain, a VL₁ and a CL₁, a third polypeptide comprising in order from the N-terminus to C-terminus a the second cytokine receptor-binding domain, a VL₂, a CH1₂ and a second Fc domain subunit, and a fourth polypeptide comprising in order from the N-terminus to C-terminus a VH₂ and a CL₂: or f) a first polypeptide comprising in order from the N-terminus to C-terminus a first Fc domain subunit, a VL₁ and a CH1₁, a second polypeptide comprising in order from the N-terminus to C-terminus a the first cytokine receptor-binding domain, a VH₁ and a CL₁, a third polypeptide comprising in order from the N-terminus to C-terminus a the second cytokine receptor-binding domain, a VH₂, a CH1₂ and a second Fc domain subunit, and a fourth polypeptide comprising in order from the N-terminus to C-terminus a VL₂ and a CL₂: or g) a first polypeptide comprising in order from the N-terminus to C-terminus a first Fc domain subunit, a VL₁ and a CL₁, a second polypeptide comprising in order from the N-terminus to C-terminus a the first cytokine receptor-binding domain, a VH₁ and a CH1₁, a third polypeptide comprising in order from the N-terminus to C-terminus a the second cytokine receptor-binding domain, a VL₂, a CH1₂ and a second Fc domain subunit, and a fourth polypeptide comprising in order from the N-terminus to C-terminus a VH₂ and a CL₂; or h) a first polypeptide comprising in order from the N-terminus to C-terminus a first Fc domain subunit, a VH₁ and a CL₁, a second polypeptide comprising in order from the N-terminus to C-terminus a the first cytokine receptor-binding domain, a VL₁ and a CH1₁, a third polypeptide comprising in order from the N-terminus to C-terminus a the second cytokine receptor-binding domain, a VH₂, a CH1₂ and a second Fc domain subunit, and a fourth polypeptide comprising in order from the N-terminus to C-terminus a VL₂ and a CL₂; wherein VH₁, VL₁, CH1₁, and CL₁ form the first target-binding domain and VH₂, VL₂, CH1₂, and CL₂ form the second target-binding domain.

In one aspect, the first target-binding domain and the second target-binding domain specifically bind to FAP and the first and second cytokine receptor subunits are subunits of the IFNγ receptor complex. In one aspect, the antigen binding molecule comprises a) a first polypeptide comprising the amino acid sequence of SEQ ID NO: 50, a second polypeptide comprising the amino acid sequence of SEQ ID NO: 48, a third polypeptide comprising the amino acid sequence of SEQ ID NO: 51 and a fourth polypeptide comprising the amino acid sequence of 25 SEQ ID NO: 49; or b) a first polypeptide comprising an amino acid sequence of SEQ ID NO: 99, a second polypeptide comprising an amino acid sequence of SEQ ID NO: 97, a third polypeptide comprising an amino acid sequence of SEQ ID NO: 100, and a fourth polypeptide comprising an amino acid sequence of SEQ ID NO: 98; or c) a first polypeptide comprising an amino acid sequence of SEQ ID NO: 103, a second polypeptide comprising an amino acid sequence of SEQ ID NO: 101, a third polypeptide comprising an amino acid sequence of SEQ ID NO: 104, and a fourth polypeptide comprising an amino acid sequence of SEQ ID NO: 102; or d) a first polypeptide comprising an amino acid sequence of SEQ ID NO: 107, a second polypeptide comprising an amino acid sequence of SEQ ID NO: 105, a third polypeptide comprising an amino acid sequence of SEQ ID NO: 108, and a fourth polypeptide comprising an amino acid sequence of SEQ ID NO: 106; or e) a first polypeptide comprising an amino acid sequence of SEQ ID NO: 99, a second polypeptide comprising an amino acid sequence of SEQ ID NO: 109, a third polypeptide comprising an amino acid sequence of SEQ ID NO: 100, and a fourth polypeptide comprising an amino acid sequence of SEQ ID NO: 110; or f) a first polypeptide comprising an amino acid sequence of SEQ ID NO: 107, a second polypeptide comprising an amino acid sequence of SEQ ID NO: 111, a third polypeptide comprising an amino acid sequence of SEQ ID NO: 113, and a fourth polypeptide comprising an amino acid sequence of SEQ ID NO: 112; or g) a first polypeptide comprising an amino acid sequence of SEQ ID NO: 99, a second polypeptide comprising an amino acid sequence of SEQ ID NO: 114, a third polypeptide comprising an amino acid sequence of SEQ ID NO: 100, and a fourth polypeptide comprising an amino acid sequence of SEQ ID NO: 115; or h) a first polypeptide comprising an amino acid sequence of SEQ ID NO: 107, a second polypeptide comprising an amino acid sequence of SEQ ID NO: 116, a third polypeptide comprising an amino acid sequence of SEQ ID NO: 108, and a fourth polypeptide comprising an amino acid sequence of SEQ ID NO: 117.

In one aspect, the first target-binding domain and the second target-binding domain specifically bind to PD-1 and the first and second cytokine receptor subunits are subunits of the IL-2 receptor complex.

In one aspect, the antigen binding molecule comprises a first polypeptide comprising an amino acid sequence of SEQ ID NO: 123, a second polypeptide comprising an amino acid sequence of SEQ ID NO: 122, a third polypeptide comprising an amino acid sequence of SEQ ID NO: 124, and a fourth polypeptide comprising an amino acid sequence of SEQ ID NO: 125.

### BRIEF DESCRIPTION OF THE FIG.S

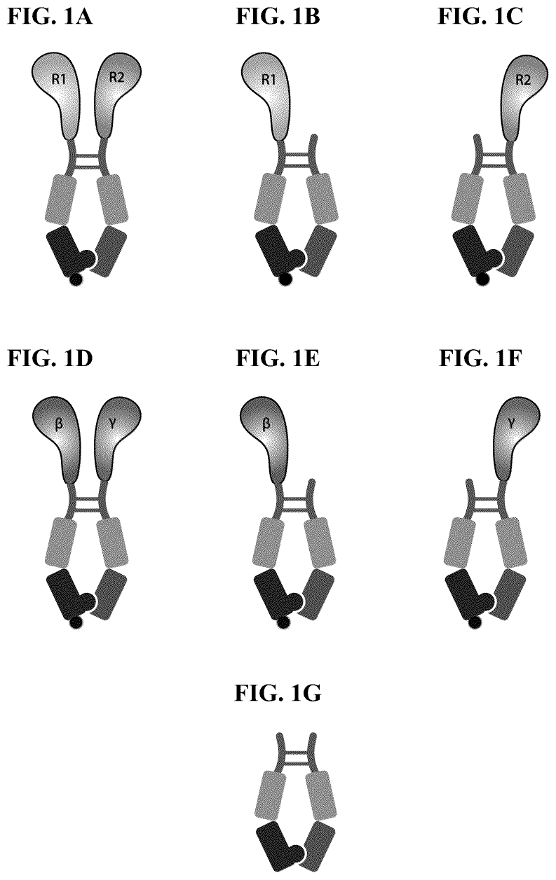

FIG. 1A-1G. Schematic representations of antigens used in llama immunization, phage display and screening for the isolation of human IFNγR1, IFNγR2, IL-2Rβ and IL-2Rγ-specific single domain antibodies. For llama immunization a heterodimer formed of the extracellular domain (ECD) of human IFNγR1 fused to biotinylated Fc knob and human IFNγR2 ECD fused to Fc hole (FIG. 1A) and a heterodimer formed of the ECD of human IL-2Rβ fused to biotinylated Fc knob and human IL-2Rγ ECD fused to Fc hole (FIG. 1D) were generated. For phage display monovalent human IFNγR1 fused to biotinylated Fc (FIG. 1B), monovalent human IFNγR2 fused to biotinylated Fc (FIG. 1C), monovalent human IL-2Rβ fused to biotinylated Fc (FIG. 1E), monovalent human IL-2Rγ fused to biotinylated Fc (FIG. 1F) and soluble Fc (FIG. 1G) were generated.

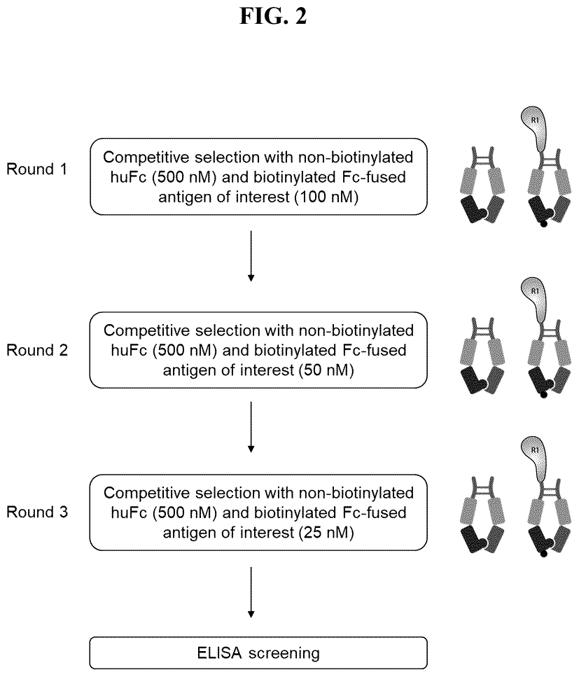

FIG. 2. Workflow for the enrichment of single domain antibodies with binding specificity for human cytokine receptor subunits by phage display.

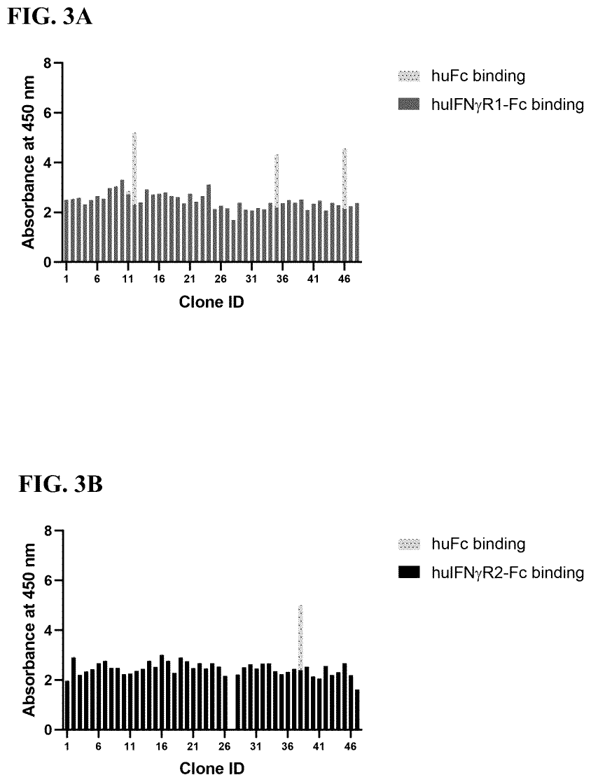

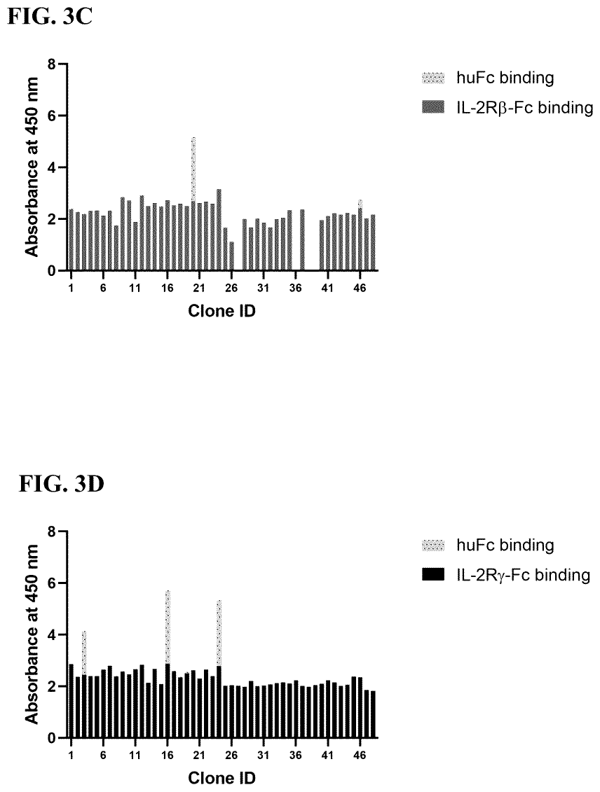

FIG. 3A-FIG. 3D. Specificity screening of single domain antibodies by ELISA. A random set of soluble VHH domains selected for human IFNγR1 specificity following three rounds of phage display were tested for binding to immobilized human IFNγR1-Fc and immobilized human Fc (FIG. 3A). VHH domains with human IFNγR2 specificity following three rounds of phage display were also tested for antigen specificity by ELISA (FIG. 3B). VHH domains selected for human IL-2Rβ specificity following three rounds of phage display were tested for binding to immobilized human IL-2Rβ-Fc and immobilized human Fc (FIG. 3C). VHH domains with human IL-2Rγ specificity following three rounds of phage display were also tested for antigen specificity by ELISA (FIG. 3D). Absorbance at 450 nm indicating the binding response to each target is represented as a stacked bar chart.

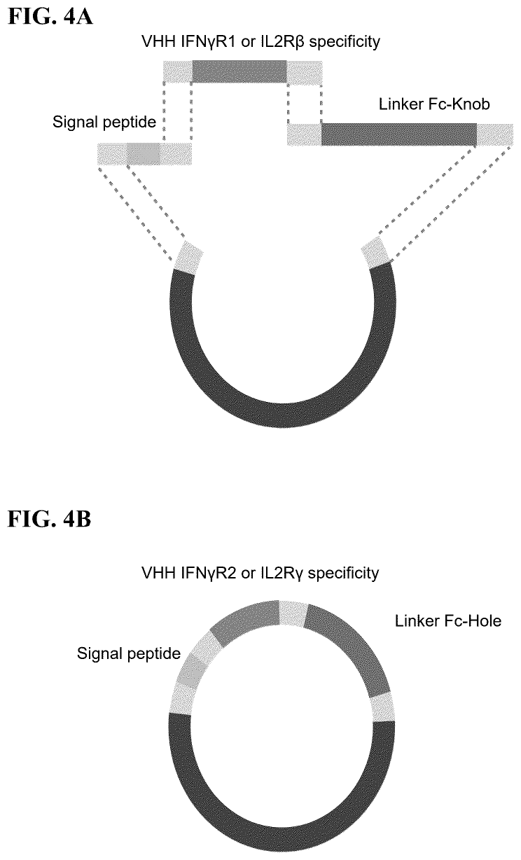

FIG. 4A-FIG. 4B. Format conversion of single domain antibody fragments. Round 3 VHH library variants with IFNγR1 or IL-2Rβ specificity were fused to Fc-Knob and cloned into a mammalian cell expression vector using Gibson cloning method (FIG. 4A). Round 3 VHH library variants with IFNγR2 or IL-2Rγ specificity were fused to Fc-hole using the same method (FIG. 4B).

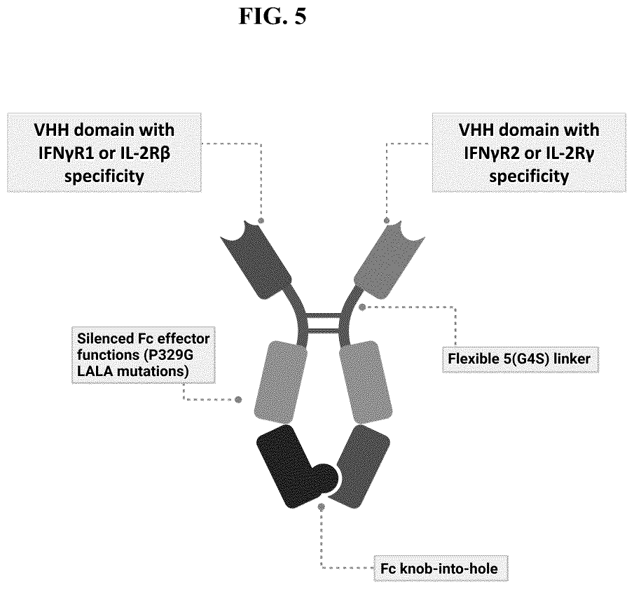

FIG. 5. Bispecific heavy chain antibody comprising IFNγR1-IFNγR2 or IL-2Rβ-IL-2Rγ VHH domain pairs. VHH domains with IFNγR1 or IL-2Rβ specificity were fused to Fc-knob and VHH domains targeting IFNγR2 or IL-2Rγ were fused to Fc-hole. Flexible 5 (G4S) linkers were used to fuse VHH moieties to Fc chains. Bispecific heavy chain antibodies were generated via knob-into-hole assembly of Fc chains. Effector functions of the Fc were silenced via incorporation of P329G LALA mutations in the CH2 domain.

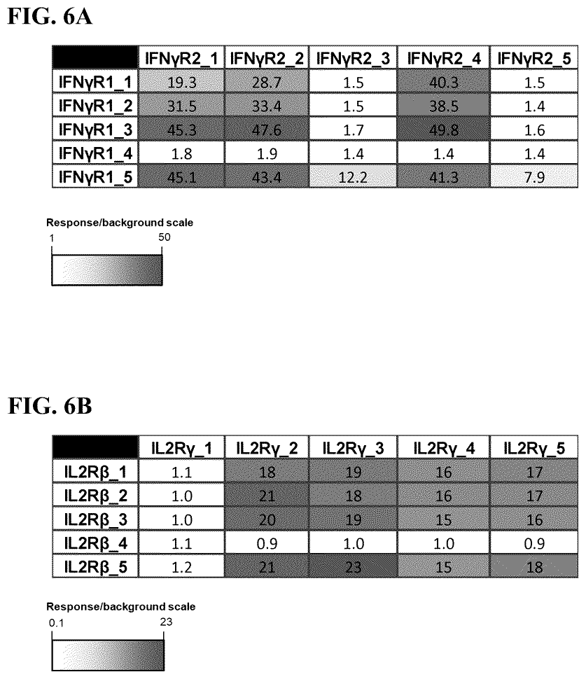

FIG. 6A-FIG. 6B. Functional screening of bispecific heavy chain antibodies comprising IFNγR1-IFNγR2 or IL-2Rβ-IL-2Rγ VHH domain pairs. HEK-Blue IFNγ cells were incubated with IFNγ agonistic bispecific heavy chain antibodies comprising IFNγR1 and IFNγR2 VHH domain pairs generated using a 5×5 bispecificity matrix (FIG. 6A). HEK-Blue IL-2 reporter cells were incubated with IL-2 agonistic bispecific heavy chain antibodies comprising IL-2Rβ and IL-2Rγ VHH pairs generated using a 5×5 bispecificity matrix (FIG. 6B). IFNγR activity and IL-2R activity was quantified by absorbance at 650 nm and fold response/background is shown for each treatment.

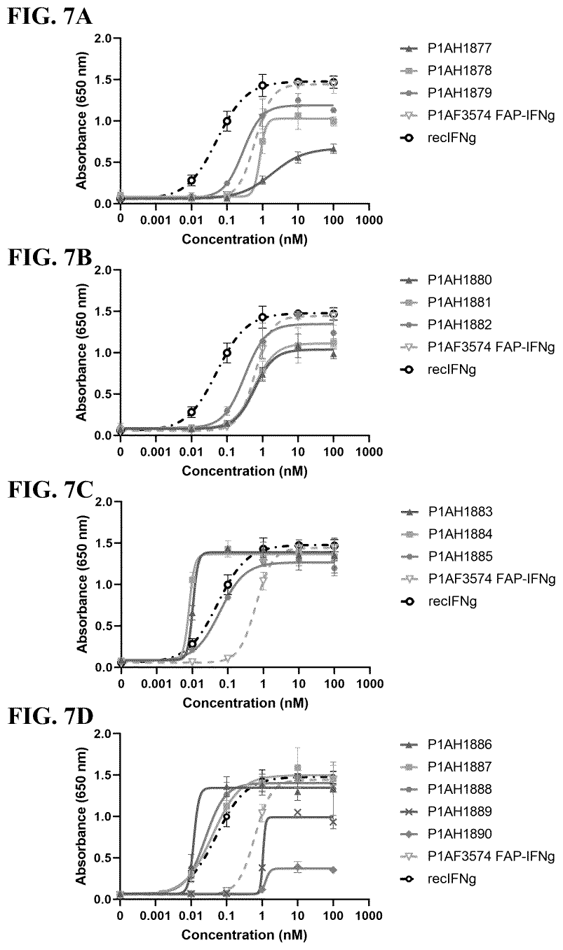

FIG. 7A-FIG. 7D. Characterisation of dose-dependent response for IFNγ agonistic heavy chain antibodies. Previously identified IFNγR agonists (FIG. 6A, Table 1) were screened for dose-dependent IFNγR activity in HEK-Blue IFNγ cells, characterized by absorbance at 650 nm. The responses of different IFNγ agonistic heavy chain antibodies, are grouped according to the anti-IFNγR1 VHH clone, i.e. within each graph the anti-IFNγR2 VHH clone is variable while the anti-IFNγR1 VHH clone remains constant: IFNγR1_1 clone (FIG. 7A), IFNγR1_2 clone (FIG. 7B), IFNγR1_3 clone (FIG. 7C) and IFNγR1_5 clone (FIG. 7D). Responses were compared to recombinant human IFNγ (black dashed line) and FAP-IFNγ (P1AF3574: light grey dashed line).

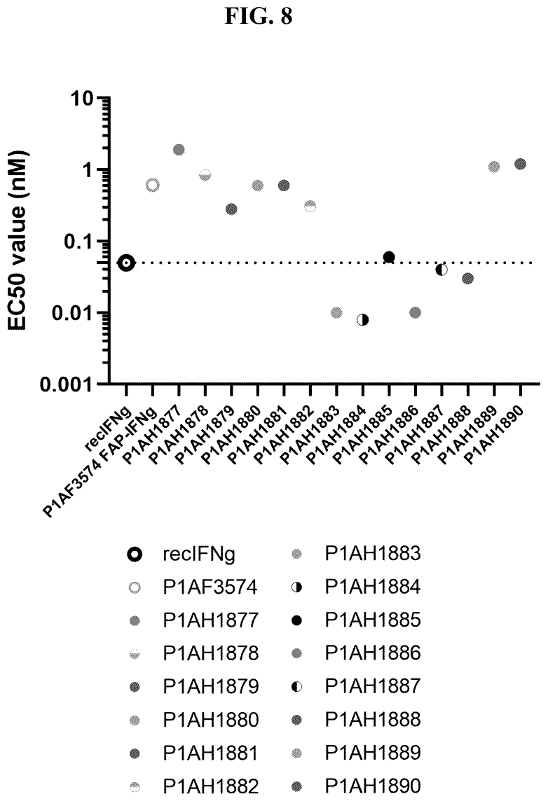

FIG. 8. Comparison of EC50 values for IFNγ agonistic heavy chain antibodies. EC50 values were derived from HEK-Blue assays (FIG. 7) using GraphPad Prism software. Values are depicted in nanomolar concentration with recombinant human IFNγ highlighted as a reference (dotted line).

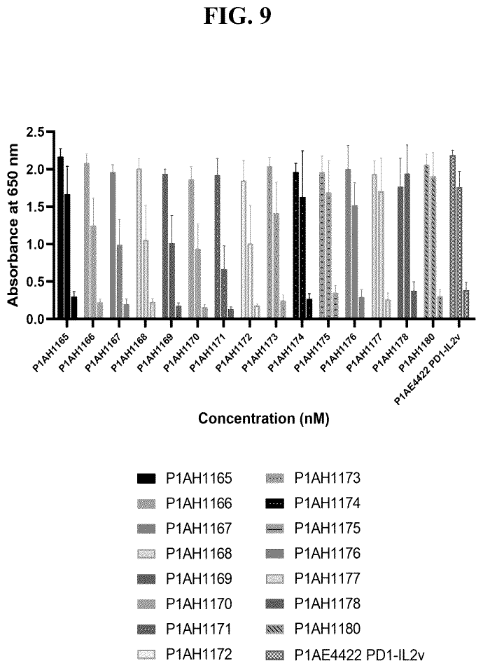

FIG. 9. Characterization of dose-dependent response for IL-2R agonistic heavy chain antibodies. Previously identified IL-2R agonists (FIG. 6B, Table 2) were screened for dose-dependent IL-2R agonism in HEK-Blue IL-2 cells. Anti-PD1-IL2v (P1AE4422) was used as a reference molecule and each molecule was incubated with reporter cells at the following concentrations: 20 nM, 0.8 nM, 0.032 nM shown from left to right. IL-2R activity was quantified by absorbance at 650 nm: mean values from technical triplicates are shown with error bars representing standard deviation.

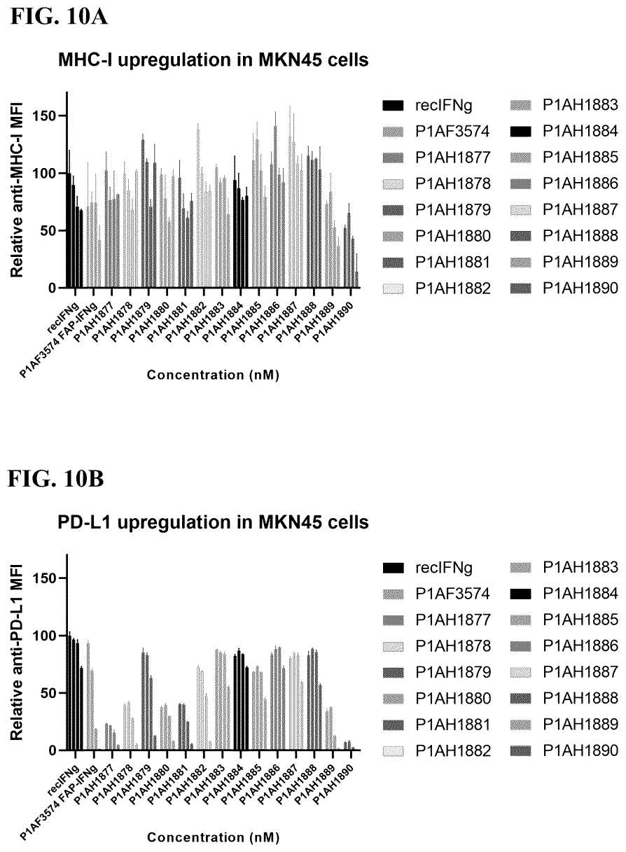

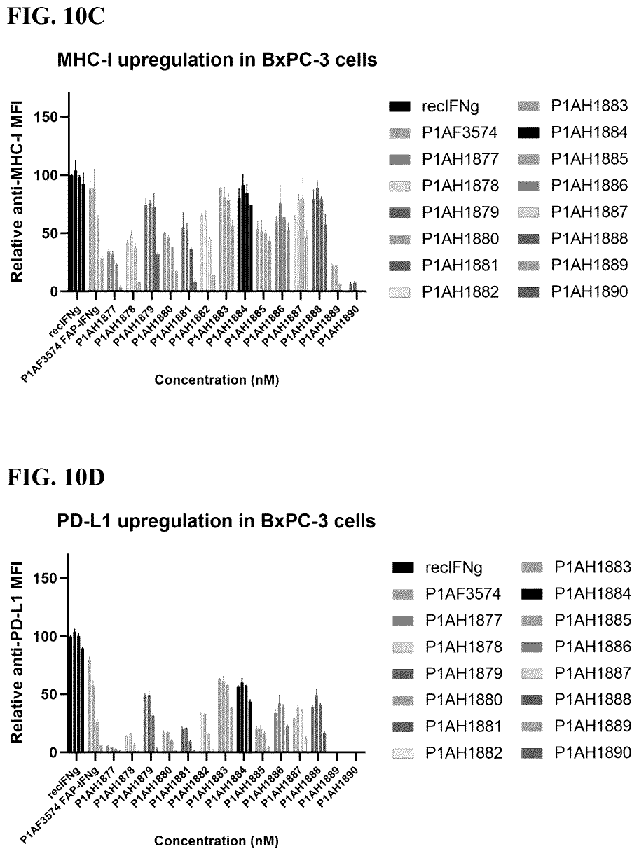

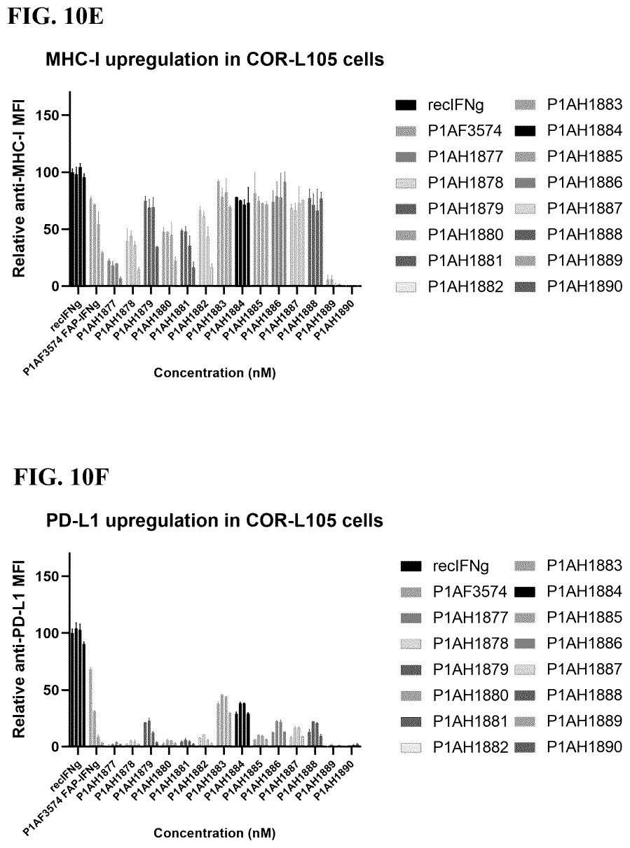

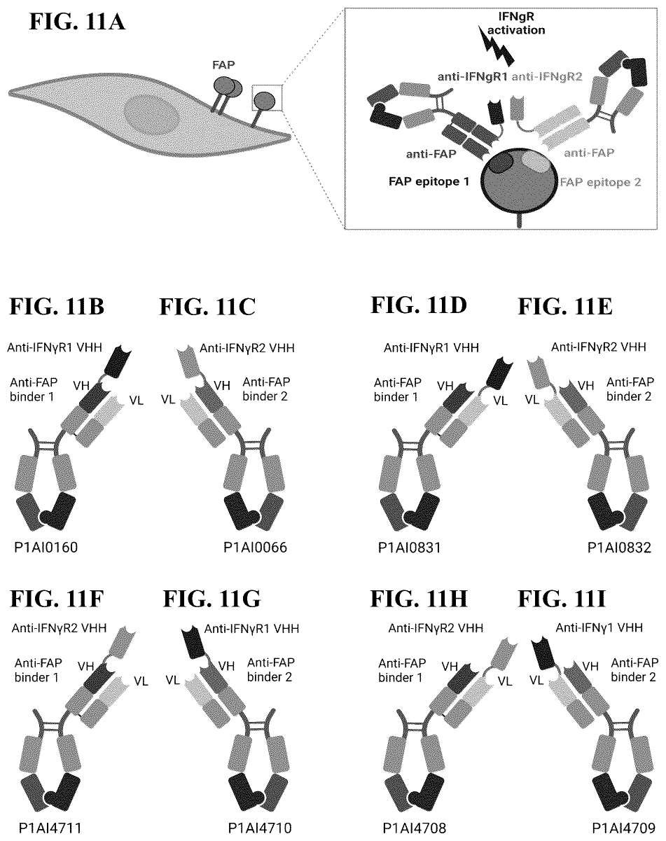

FIG. 10A-FIG. 10F. MHC-I and PD-L1 expression on tumor cells following 72 h treatment of IFNγ agonists. Previously identified IFNγR agonists (P1AH1877-P1AH1890) and reference molecules (recombinant human IFNγ and FAP-IFNγ) were incubated with tumor cells at the following concentration of treatment: 100, 10, 1 and 0.1 nM shown from left to right for each treatment. Measurements were blank-subtracted and normalized to recombinant human IFNγ response at the highest concentration (100) nM). Responses are depicted for MHC-I expression on MKN45 cells (FIG. 10A), PD-L1 expression on MKN45 cells (FIG. 10B), for MHC-I expression on Bxpc3 cells (FIG. 10C), PD-L1 expression on Bxpc3 cells (FIG. 10D), MHC-I expression on CorL105 cells (FIG. 10E) and PD-L1 expression on CorL105 cells (FIG. 10F).

FIG. 11A-FIG. 11I. Concept of split dual-targeted IFNγR agonists. FAP-dependent biparatopic assembly of split IFNγ mimetics (FIG. 11A). Note that for illustrative simplification, only one monomer of the FAP dimer is represented in the inset. VHH domains with IFNγR1 and IFNγR2 specificity were fused to the N-terminus of two different anti-FAP binding domains, via VH or VL fusion points, resulting in eight configurations within the permutation space: IFNγR1-VHH fused to the VH of FAP binder 1 (FIG. 11B), IFNγR2-VHH fused to the VH of FAP binder 2 (FIG. 11C), IFNγR1-VHH fused to the VL of FAP binder 1 (FIG. 11D), IFNγR2-VHH fused to the VL of FAP binder 2 (FIG. 11E), IFNγR2-VHH fused to the VH of FAP binder 1 (FIG. 11F), IFNγR1-VHH fused to the VH of FAP binder 2 (FIG. 11G), IFNγR2-VHH fused to the VL of FAP binder 1 (FIG. 11H) and IFNγR1-VHH fused to the VL of FAP binder 2 (FIG. 11I). All molecules have the same Fc characteristics as described in FIG. 5.

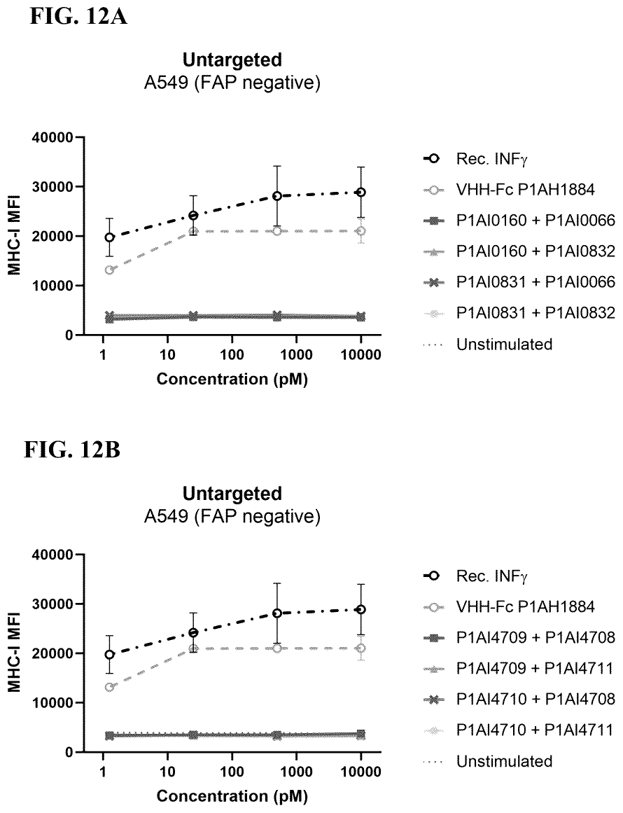

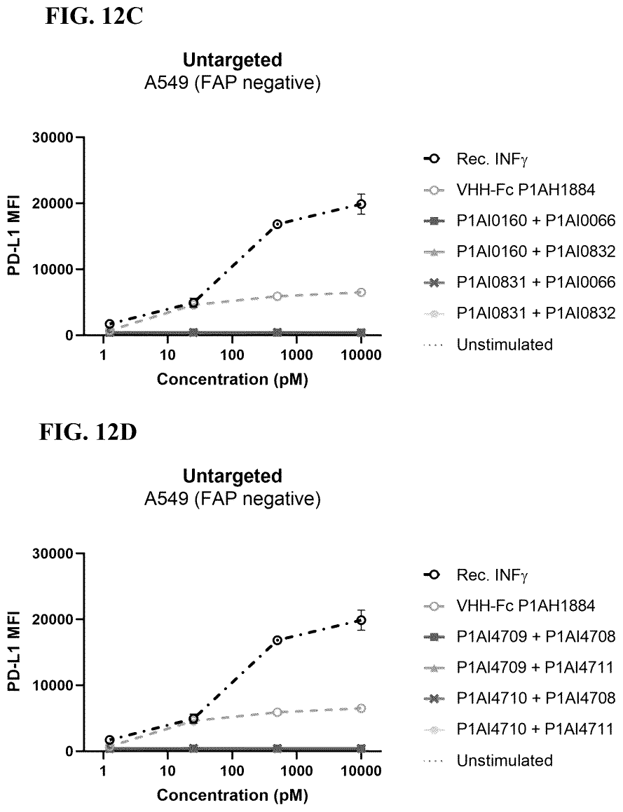

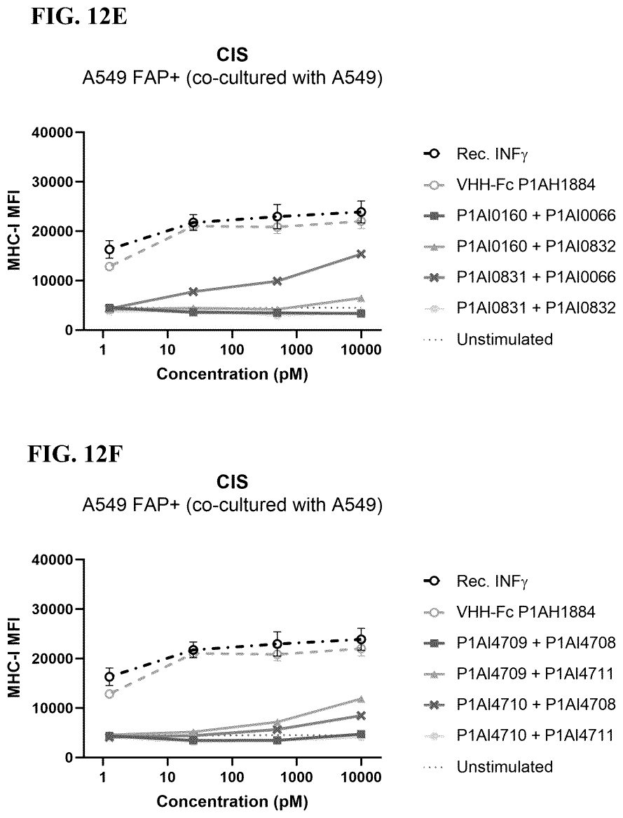

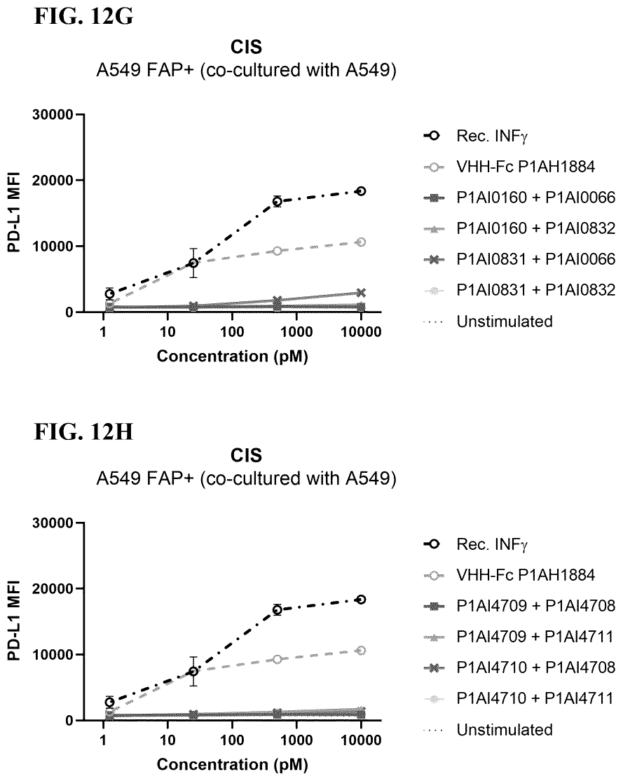

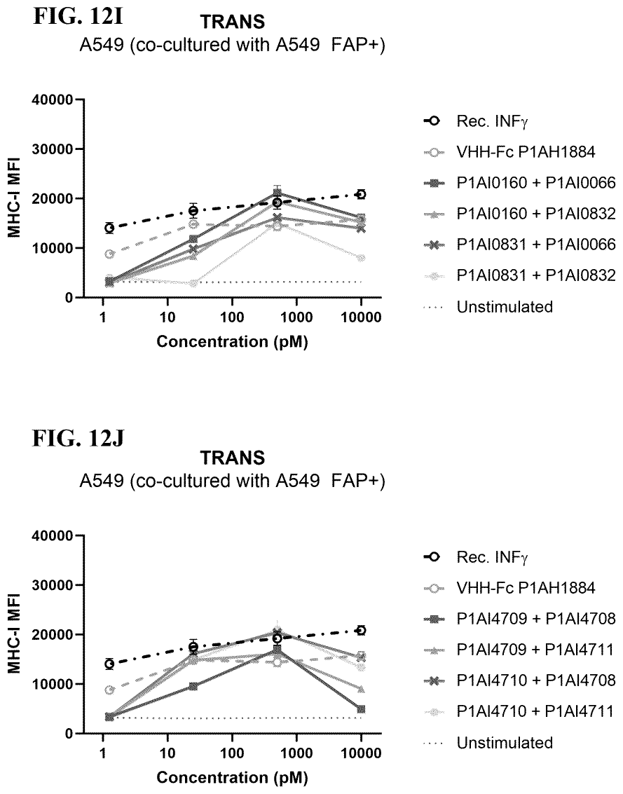

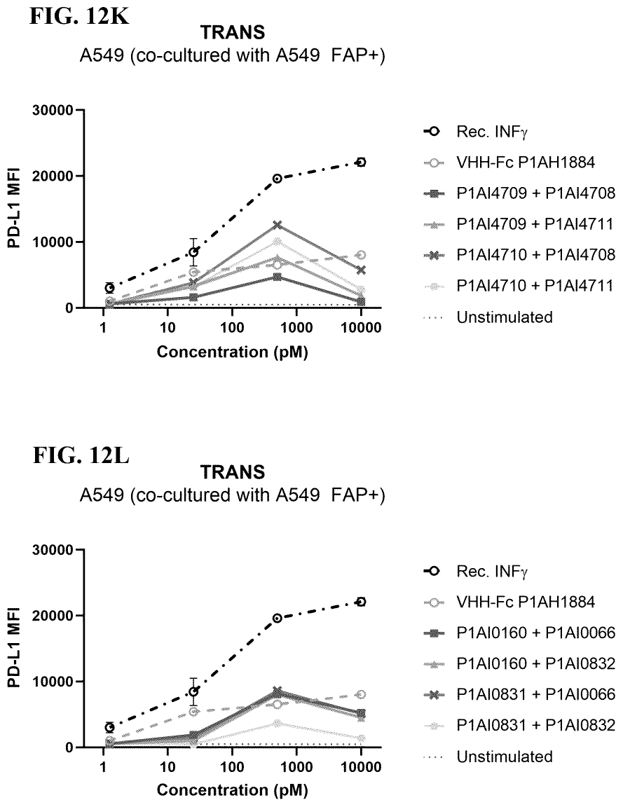

FIG. 12A-FIG. 12L. Assessment of IFNγR activity mediated by FAP-dependent split IFNγR agonists. The eight split IFNγ mimetics (FIG. 11B-I) were paired in biparatopic assembly combinations and tested for IFNγR agonism characterized by MHC-I and PD-L1 upregulation. FAP-negative A549 cells were tested for untargeted, non-specific MHC-I (FIG. 12A, FIG. 12B) and PD-L1 upregulation (FIG. 12C, FIG. 12D) by split IFNγ mimetics. A549 FAP-positive cells were co-cultured with differentially labeled A549 FAP-negative cells to assess FAP-dependent activity of IFNγ mimetics in cis and trans: MHC-I upregulation in cis (FIG. 12E and FIG. 12F), PD-L1 upregulation in cis (FIG. 12G and FIG. 12H), MHC-I upregulation in trans (FIG. 12I and FIG. 12J) and PD-L1 upregulation in trans (FIG. 12K and FIG. 12L). Recombinant IFNγ and the IFNγ agonistic heavy chain antibody P1AH1884 (as depicted in FIG. 5) were used as a reference. Median fluorescent intensity (MFI) of MHC-I and PD-L1 expression levels were analyzed in FlowJo: mean values from technical duplicates are shown with error bars representing standard deviation.

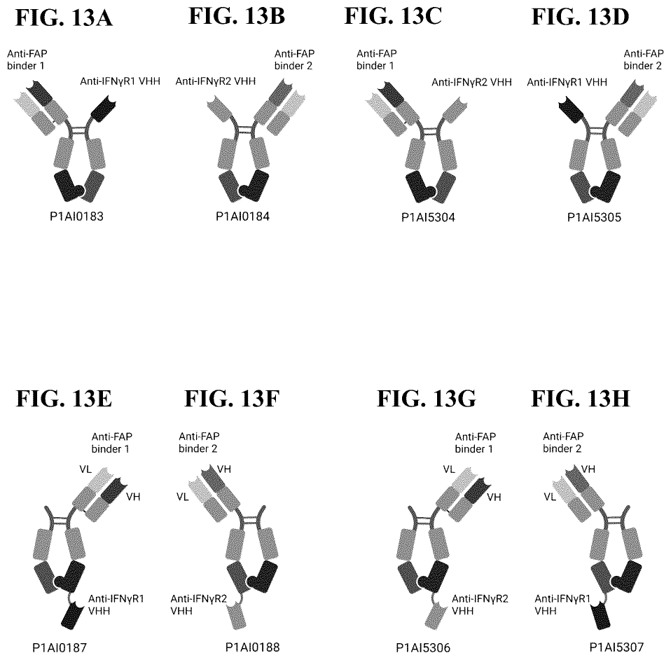

FIG. 13A-FIG. 13H. Additional formats of split dual-targeted IFNγR agonists. VHH domains with IFNγR1 and IFNγR2 specificity were distanced from the anti-FAP binders via fusion to the N-terminus of the opposite Fc chain, resulting in four configurations: IFNγR1 and FAP binder 1 (FIG. 13A), IFNγR2 and FAP binder 2 (FIG. 13B), IFNγR2 and FAP binder 1 (FIG. 13C) and IFNγR1 and FAP binder 2 (FIG. 13D). Alternatively, the VHH domains with IFNγR1 and IFNγR2 specificity were distanced from the anti-FAP binders via fusion to the C-terminus of the same Fc chain, resulting in four configurations: IFNγR1 and FAP binder 1 (FIG. 13E), IFNγR2 and FAP binder 2 (FIG. 13F), IFNγR2 and FAP binder 1 (FIG. 13G) and IFNγR1 and FAP binder 2 (FIG. 13H). The anti-FAP binders in FIG. 13E-H comprise CrossFab VH/VL engineering (binder 1) and CH1-CL charges (binder 2) to subsequently allow for additional pairings. All molecules have the same Fc characteristics as described in FIG. 5.

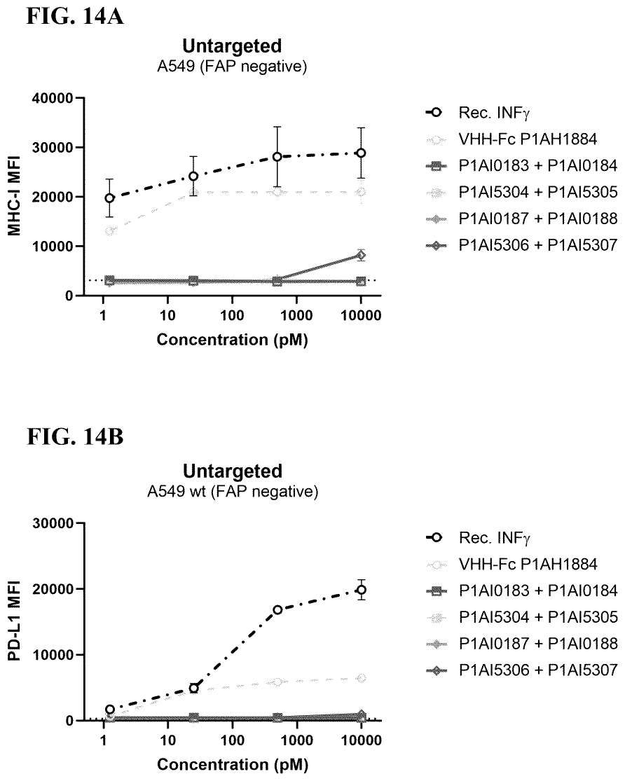

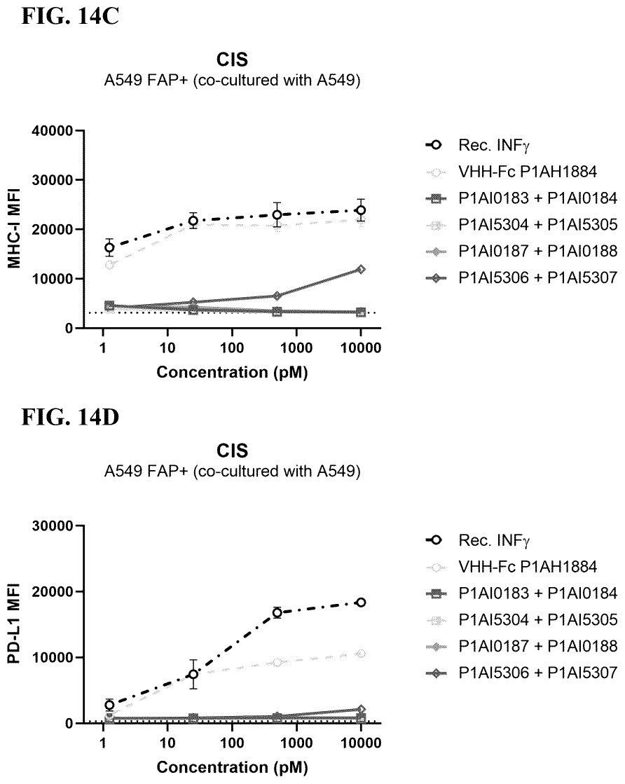

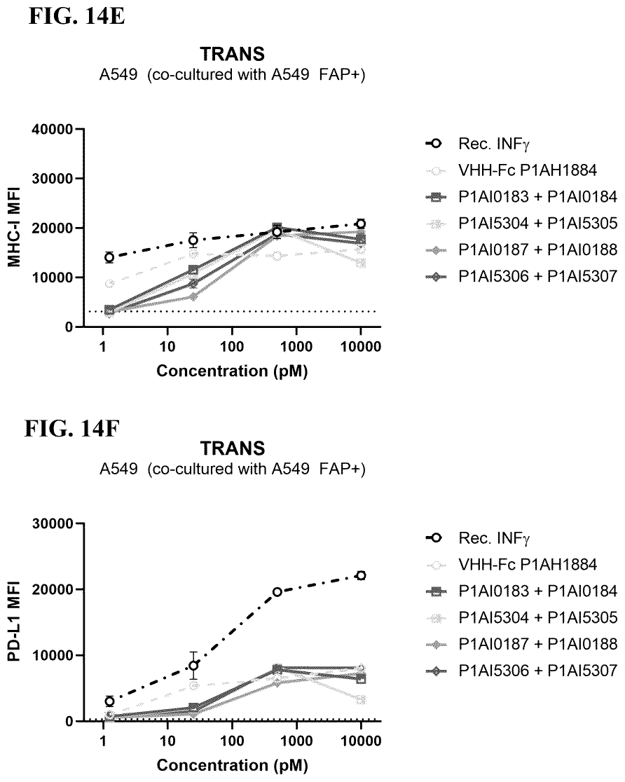

FIG. 14A-FIG. 14F. Assessment of IFNγR activity mediated by additional FAP-dependent split IFNγR agonists. Four combinations of molecules depicted in FIG. 13 and reference molecules were tested for IFNγR agonism characterized by MHC-I and PD-L1 upregulation. A549 cells lacking FAP expression were tested for FAP-independent MHC-I upregulation (FIG. 14A) and PD-L1 upregulation (FIG. 14B) of split IFNγ mimetics. A549 FAP-positive cells were co-cultured with differentially labeled A549 FAP-negative cells to assess FAP-dependent activity of IFNγ mimetics by MHC-I upregulation in cis (FIG. 14C), PD-L1 upregulation in cis (FIG. 14D), MHC-I upregulation in trans (FIG. 14E) and PD-L I upregulation in trans (FIG. 14F). Recombinant IFNγ and an IFNγ agonistic heavy chain antibody (P1AH1884) were used as reference molecules. Median fluorescent intensity (MFI) of MHC-I and PD-L1 expression levels were analyzed in FlowJo: mean values from technical duplicates are shown with error bars representing standard deviation.

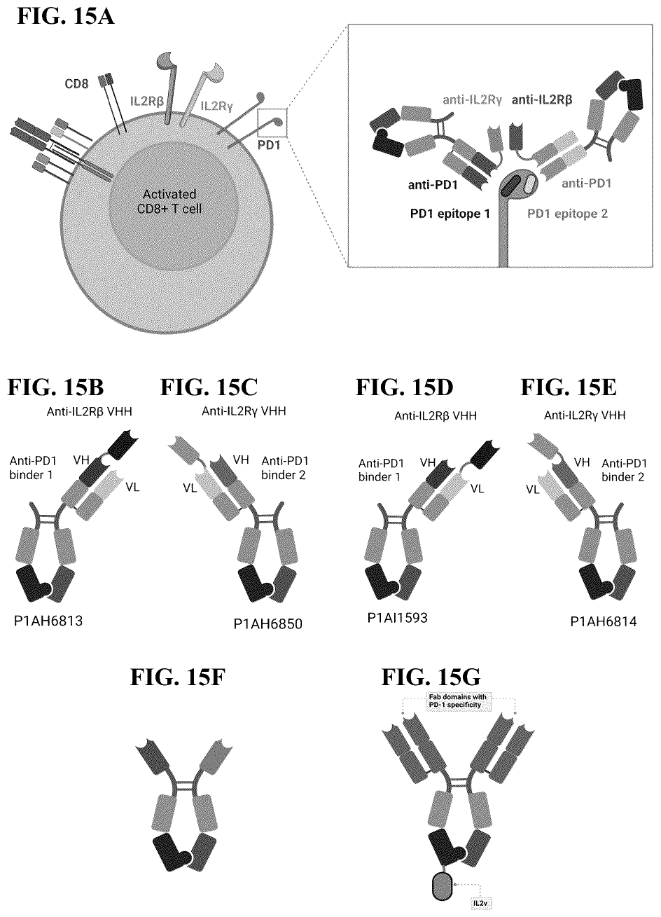

FIG. 15A-FIG. 15G. Concept of split dual-targeted IL-2 agonists. PD-1-dependent biparatopic assembly of split IL-2 mimetics (FIG. 15A). VHH domains with IL-2Rβ and IL-2Rγ specificity were fused to the N-terminus of two different anti-PD-1 binding domains, via VH or VL fusion points: IL-2Rβ-VHH fused to the VH of PD-1 binder 1 (FIG. 15B), IL-2Rγ-VHH fused to the VL of PD-1 binder 2 (FIG. 15C), IL-2Rβ-VHH fused to the VL of PD-1 binder 1 (FIG. 15D) and IL-2Rγ-VHH fused to the VH of PD-1 binder 2 (FIG. 15E). An IL-2 agonistic heavy chain antibody (FIG. 15F) and PD-1-IL2v (FIG. 15G) served as reference molecules. All molecules have the same Fc characteristics as described in FIG. 5.

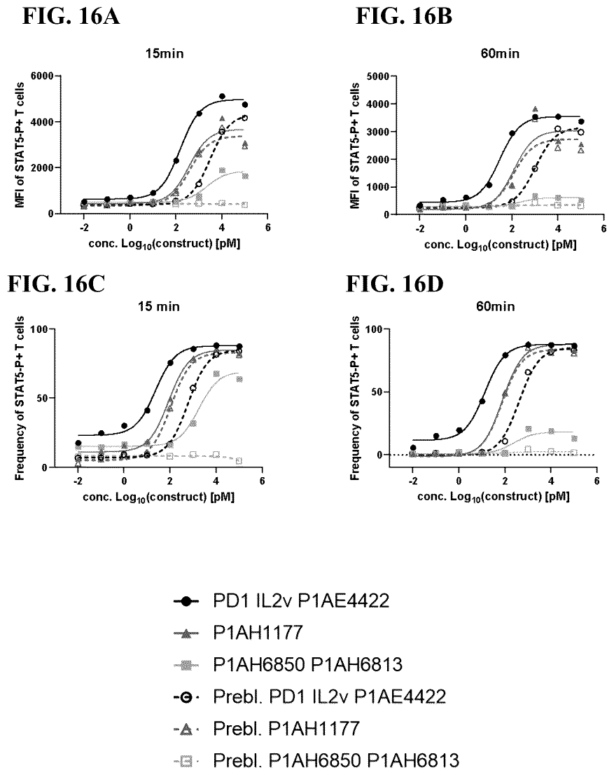

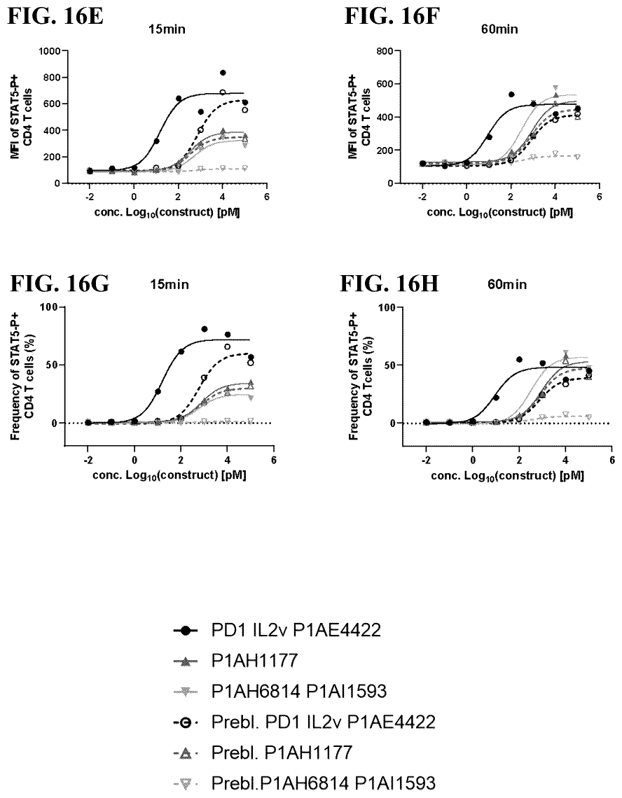

FIG. 16A-FIG. 16H. Phosphorylation of STAT5 on CD4 T cells after 15 min and 60 min incubation with IL-2R agonists. Activated T cells expressing PD-1 (PD-1⁺ subset) and activated T cells preblocked with anti-PD-1 antibodies (PD-1⁻ subset) were differentially labeled and treated with two combinations of split dual-targeted IL-2 mimetics: P1AH6850+P1AH6813 (FIG. 16A-FIG. 16D), P1AH6814+P1AI1593 (FIG. 16E-FIG. 16H). The IL-2 mimetic heavy chain antibody (P1AH1177) and PD1-IL2v (P1AE4422) were used as reference molecules. MFI and frequency of STAT5-P⁺ cells were measured by FACS and responses were shown for PD-1⁺ subset (solid line) and PD-1-subset (dashed line).

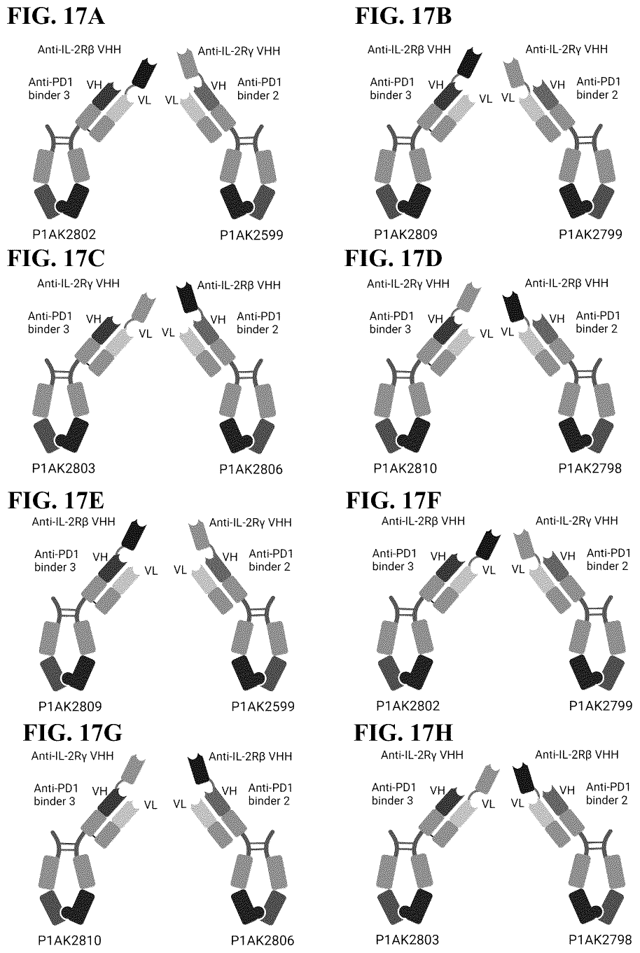

FIG. 17A-FIG. 17H. Concept of split PD-1-targeted IL-2R agonists with alternative anti-PD-1 Fab binders. VHH domains with IL-2Rβ and IL-2Rγ specificity were fused to the N-terminus of two different anti-PD-1 binding domains, via VH or VL fusion points, resulting in eight individual format configurations (P1AK2599; P1AK2798: P1AK2799: P1AK2802; P1AK2803: P1AK2806: P1AK2809; P1AK2810) that can be paired to produce the eight functional assemblies depicted: P1AK2802+P1AK2599 (FIG. 17A), P1AK2809+P1AK2799 (FIG. 17B), P1AK2803+P1AK2806 (FIG. 17C), P1AK2810+P1AK2798 (FIG. 17D), P1AK2809+P1AK2599 (FIG. 17E), P1AK2802 P1AK2 799 (FIG. 17F), P1AK2810+P1AK2806 (FIG. 17G) and P1AK2803+P1AK2798 (FIG. 17H).

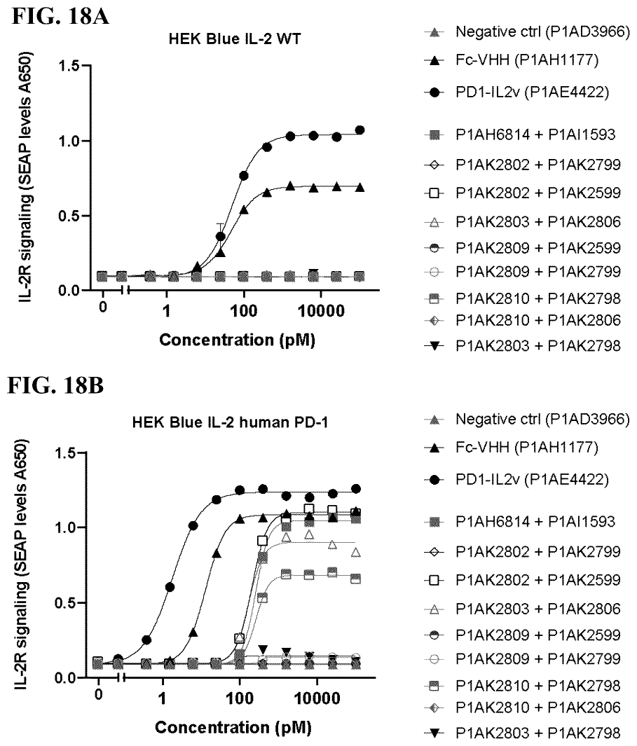

FIG. 18A-FIG. 18B. Functional activity of PD-1-targeted IL-2R agonists with alternative anti-PD-1 Fab binders. Test compounds were incubated with HEK Blue IL-2 wt cells (FIG. 18A) or HEK Blue IL-2 human PD-1 cells (FIG. 18B) for 21 h at 37° C. and 5% CO₂. PD1-IL2v (P1AE4422) and Fc-VHH (P1AH1177) were used as reference. A non-binding DP47 antibody (P1AD3966) was used as negative control. IL-2R signaling was measured via absorbance at 650 nm using QUANTI-Blue reagent. Shown are mean absorbance values+/−SEM of technical duplicates for each tested molecule concentration.

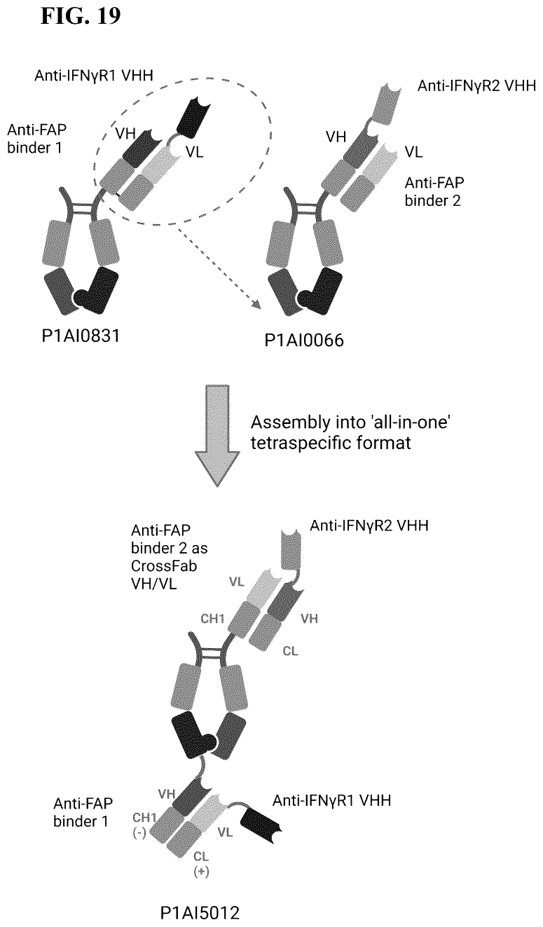

FIG. 19. Schematic representation of the ‘all-in-one’ format design. A pair of biparatopic split IFNγ mimetic molecules (P1AI0831 and P1AI0066) showing both potent cis and trans activities was formatted into a single molecule format to test for FAP-specific activity. One of the anti-FAP binding domains was grafted onto the C-terminus of the Fc in a bid to achieve sufficient molecular spacing between the IFNγ mimetic VHH pair to avoid FAP-independent IFNγR activation. CrossFab VH/VL engineering and CH1-CL charges were incorporated to generate the tetraspecific format (P1AI5012), with the Fc displaying the same characteristics described in FIG. 5.

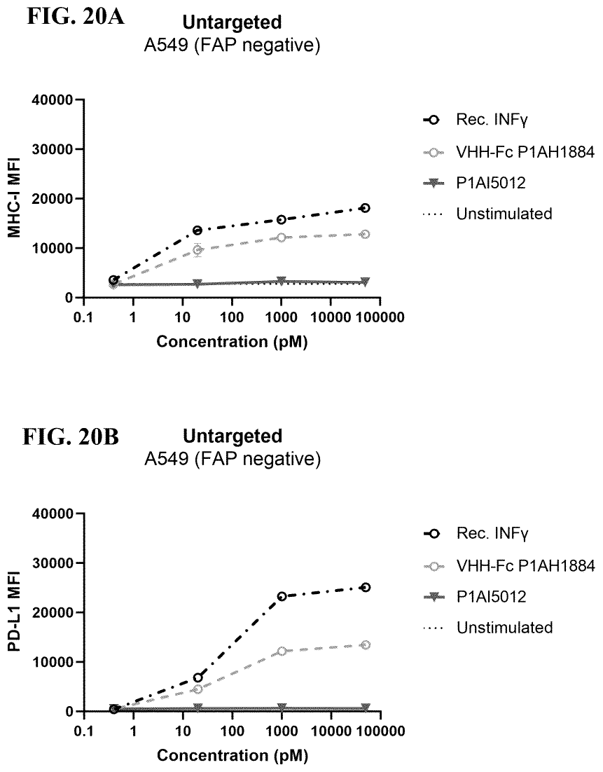

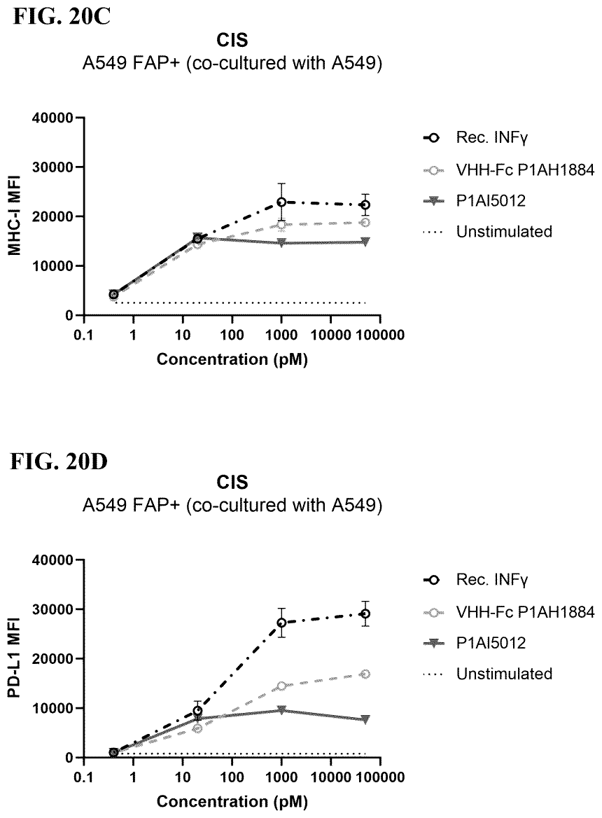

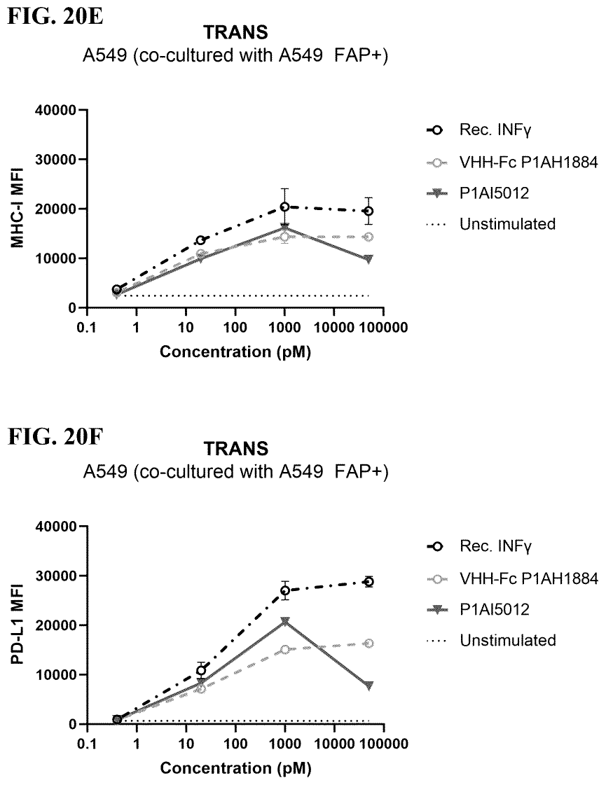

FIG. 20-FIG. 20F. Assessment of FAP-specific IFNγR activity mediated by the ‘all-in-one’ IFNγ mimetic. FAP-negative A549 cells were tested for untargeted, non-specific MHC-I upregulation (FIG. 20A) and PD-L1 upregulation (FIG. 20B) of an ‘all-in-one’ IFNγ mimetic (P1AI5012). A549 FAP-positive cells were co-cultured with differentially labeled A549 FAP-negative cells to assess FAP-dependent MHC-I upregulation of the IFNγ mimetic in cis (FIG. 20C), PD-L1 upregulation in cis (FIG. 20D), MHC-I upregulation in trans (FIG. 20E) and PD-L1 upregulation in trans (FIG. 20F). Recombinant IFNγ and the IFNγ agonistic heavy chain antibody P1AH1884 were used as a reference. Median fluorescent intensity (MFI) of MHC-I and PD-L1 expressions were analyzed in FlowJo: mean values from technical duplicates are shown with error bars representing standard deviation.

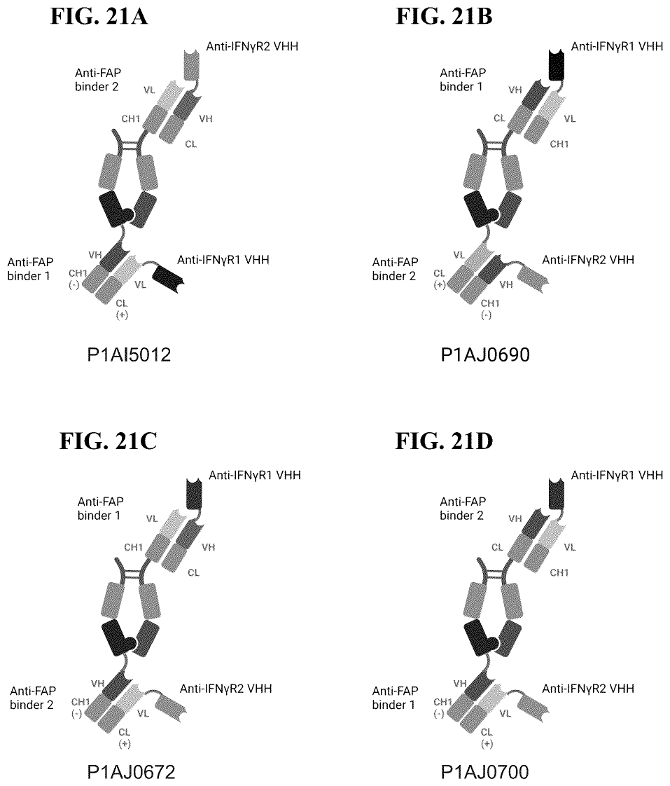

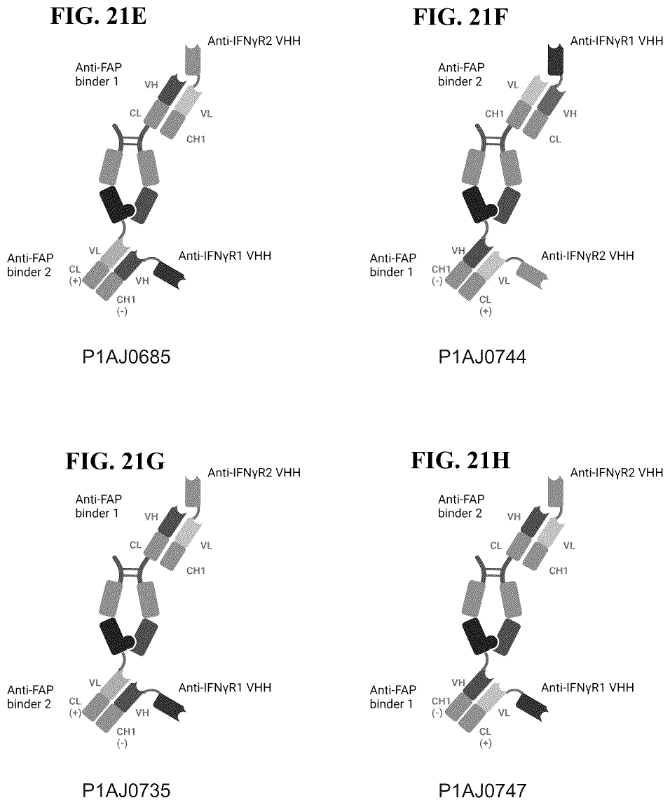

FIG. 21A-FIG. 21H. Schematic representation of additional formats of “all-in-one” molecules. Variants of the all-in-one molecule P1AI5012 (FIG. 21A) which differ in agonistic VHH pair, the position of the VHHs, respectively on the VH or VL of the Fab domains, different position for the FAP binders. P1AJ0690 (FIG. 21B), P1AJ0672 (FIG. 21C), P1AJ0700 (FIG. 21D), P1AJ0685 (FIG. 21E), P1AJ0744 (FIG. 21F), P1AJ0735 (FIG. 21G) and P1AJ0747 (FIG. 21H).

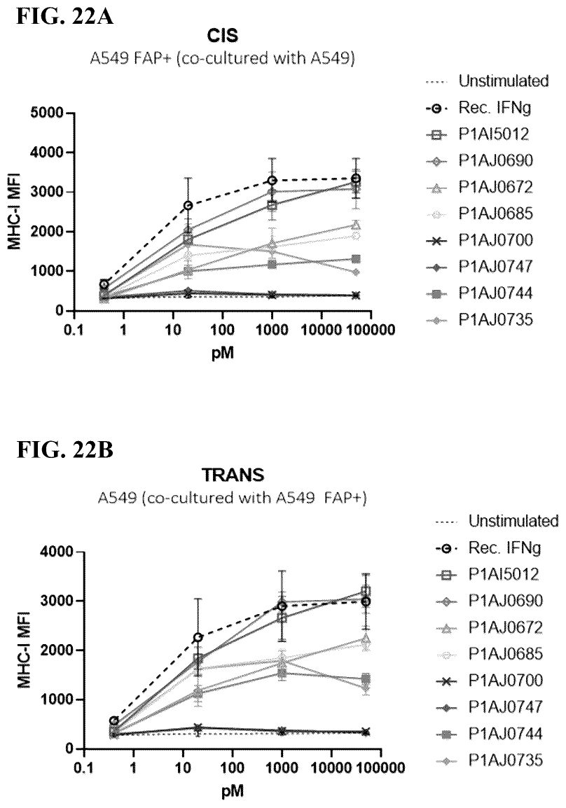

FIG. 22A-FIG. 22B. MHC-I expression on tumor cells following 72 h treatment of IFNγ agonists. Previously identified IFNγR agonists (P1AI5012, P1AJ0690, P1AJ0672, P1AJ0685, P1AJ0700, P1AJ0747, P1AJ0744, and P1AJ0735 and reference molecule (recombinant human IFNγ) were incubated with tumor cells. A549 FAP-positive cells were co-cultured with differentially labeled A549 FAP-negative cells to assess FAP-dependent activity of IFNγ mimetics in cis and trans: MHC-I upregulation in cis (FIG. 22A) and MHC-I upregulation in trans (FIG. 22B). Median fluorescent intensity (MFI) of MHC-I expression levels were analyzed in FlowJo; mean values from technical duplicates are shown with error bars representing standard deviation.

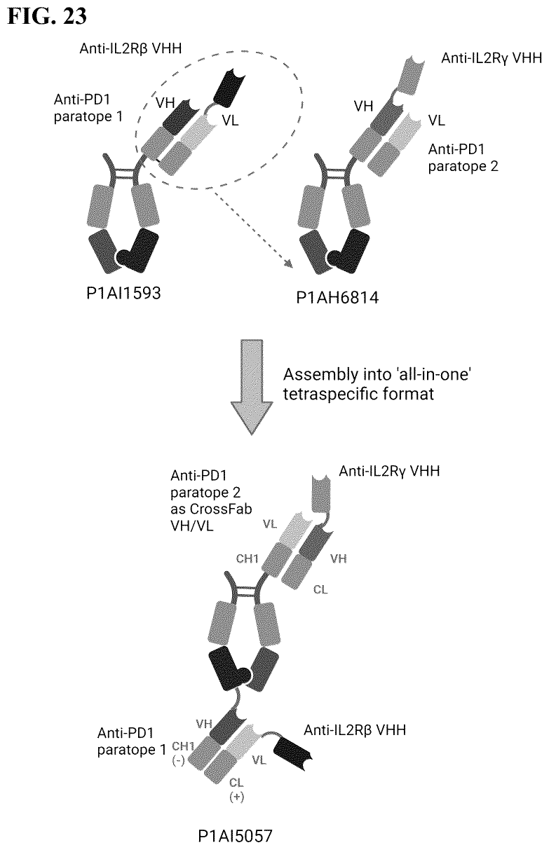

FIG. 23. Schematic representation of the ‘all-in-one’ format design. A pair of biparatopic split IL-2 mimetic molecules (P1AI1593 and P1AH6814) with potent PD1-mediated cis activity was formatted into a single molecule format (P1AI5057) to test for PD1-restricted activity. One set of the anti-PD1 and anti-IL2Rβ binding domains was grafted onto the C-terminus of the Fc in a bid to achieve sufficient molecular spacing between the IL-2 mimetic VHH pair to impair PD1-independent IL-2R activation. To generate the tetraspecific format, crossFab VH/VL engineering and CH1-CL charges were incorporated in the Fab domains and knob-into-hole mutations were present in the Fc. Effector functions of the Fc were silenced via incorporation of P329G LALA mutations in the CH2 domain.

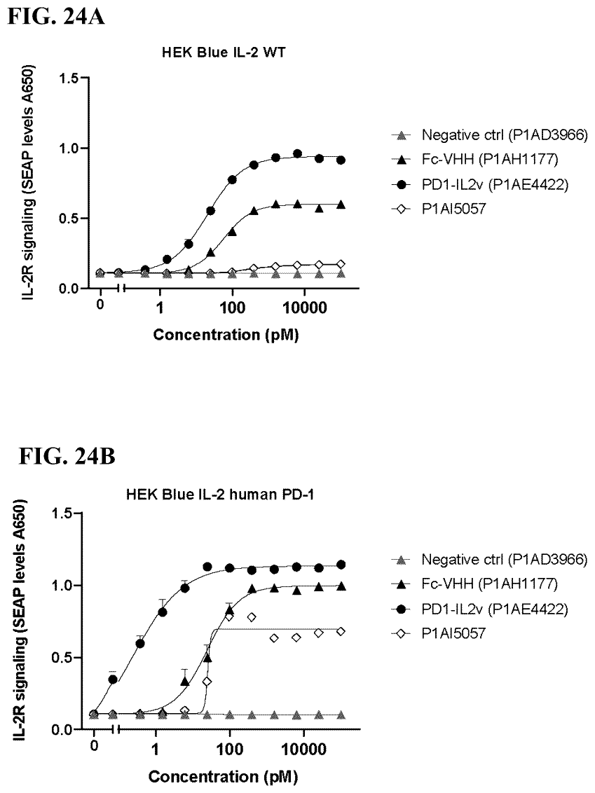

FIG. 24A-FIG. 24B. Functional activity of all-in-one PD-1 targeted IL2R agonist. Test compounds were incubated with HEK Blue IL-2 wt (FIG. 24A) or HEK Blue IL-2 human PD-1 (FIG. 24B) cells for 20 h at 37° C. and 5% CO₂. PD1-IL2v (P1AE4422) and Fc-VHH (P1AH1177) were used as reference. A non-binding DP47 antibody (P1AD3966) was used as negative control. IL2R signaling was measured via absorbance at 650 nm using QUANTI-Blue reagent. Shown are mean absorbance values+/−SEM of technical duplicates for each tested molecule concentration.

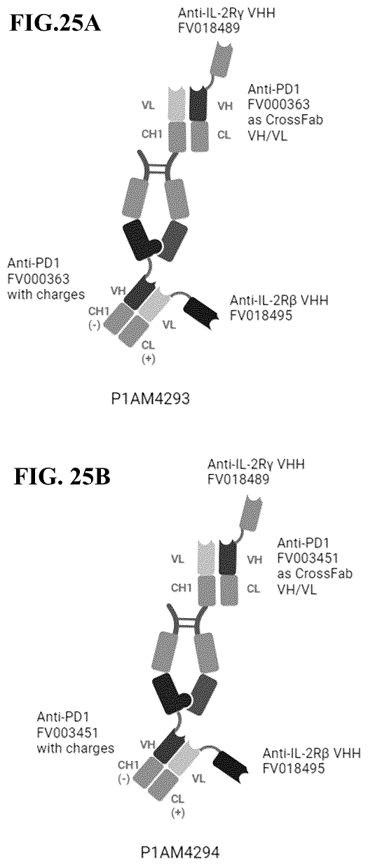

FIG. 25A-FIG. 25B: Concept of PD-1-targeted IL-2R agonists with monoparatopic assembly onto PD-1. VHH domains with IL-2Rβ and IL-2Rγ specificity were fused to the N-termini of two anti-PD1 binding domains with the same paratopes, via VH or VL fusion points. P1AM4293 (FIG. 25A) comprises two anti-PD1 binding domains with the FV000363 paratope (PD1 binder 1:0376 binder), P1AM4294 (FIG. 25B) comprises two anti-PD1 binding domains with the FV003451 paratope (PD1 binder 2:1040 binder).

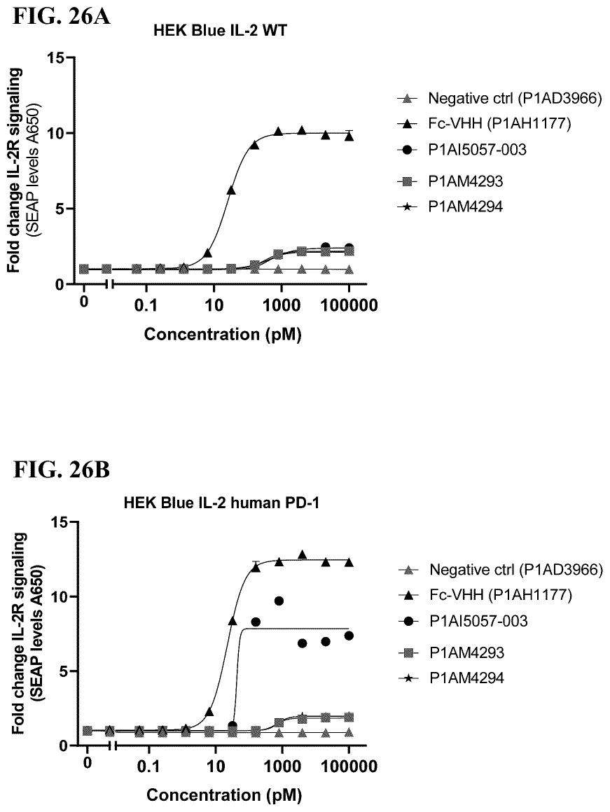

FIG. 26A-FIG. 26B: Functional activity of monoparatopic vs biparatopic PD-1-targeted IL-2R agonists. Test compounds were incubated with HEK Blue IL-2 wt (FIG. 26A) or HEK Blue IL-2 human PD-1 (FIG. 26B) cells for 18 h at 37° C. and 5% CO₂. Fc-VHH (P1AH1177) was used as reference. A non-binding DP47 antibody (P1AD3966) was used as negative control. IL-2R signaling was measured via absorbance at 650 nm using QUANTI-Blue reagent. Shown are fold change in IL-2R signaling over background+/−SEM of technical duplicates for each tested molecule concentration.

### DETAILED DESCRIPTION OF THE INVENTION

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well known in the art. Generally, nomenclatures used in connection with, and techniques of biochemistry, enzymology, molecular, and cellular biology, microbiology, genetics and protein and nucleic acid chemistry and hybridization described herein are those well-known and commonly used in the art.

The terms “a”, “an” and “the” generally include plural referents, unless the context clearly indicates otherwise.

As used herein, the terms “first”, “second”, “third” or “fourth” with respect to binding molecules, epitopes, polypeptides etc., are used for convenience of distinguishing when there is more than one of each type of moiety. Use of these terms is not intended to confer a specific order or orientation of the moiety unless explicitly so stated.

The term “antigen binding molecule” as used herein refers to a polypeptide molecule (composed of one or more polypeptide chains) that is capable of binding to an antigen. A binding molecule may be derived from an antibody, and typically comprises an antigen binding domain.

The term “antibody” herein is used in the broadest sense and encompasses various antibody structures, including but not limited to monoclonal antibodies, polyclonal antibodies, multispecific antibodies (e.g. bispecific antibodies), heavy-chain antibodies, antibody fragments and antigen binding molecules so long as they exhibit the desired antigen-binding activity.

The term “heavy chain antibody” and “heavy chain-only antibody” and “HCAb” as used herein refer to antibodies devoid of light chains.

The terms “full-length antibody,” “intact antibody,” and “whole antibody” are used herein interchangeably to refer to an antibody having a structure substantially similar to a native antibody structure.

An “antibody fragment” refers to a molecule other than an intact antibody that comprises a portion of an intact antibody that binds the antigen to which the intact antibody binds. Examples of antibody fragments include but are not limited to Fv, Fab, cross-Fab, Fab′, Fab-SH, F (ab′)2, diabodies, linear antibodies, single-chain antibody molecules (e.g. scFv and scFab), single-domain antibodies, and multispecific antibodies formed from antibody fragments. For a review of certain antibody fragments, see Hollinger and Hudson, Nature Biotechnology 23:1126-1136 (2005).

A “single-domain antibody” refers to an antibody fragment consisting of a single monomeric antibody variable domain such as VHHs, nanobodies, VNARs derived from sharks, autonomous VH domains or autonomous VL domains. Single-domain antibodies provide an antigen-binding site which specifically binds to an epitope, i.e. the antigen binding-site is formed solely by the single-domain antibody.

A “VHH” or “VHH domain” or “nanobody” refers to a single-domain antibody derived from the variable domains of heavy chain antibodies from camelids, e.g. camel, dromedary, llama, alpaca, etc. (See Nguyen V. K. et al., 2000, The EMBO Journal, 19, 921-930; Muyldermans S., 2001, J Biotechnol., 74, 277-302 and for review Vanlandschoot P. et al., 2011, Antiviral Research 92, 389-407). The antigen-binding site of VHHs is devoid of light chain variable domain. A VHH domain may be humanized.

An “antigen binding domain” as used herein refers to a domain that specifically binds to a target antigen. The term in particular refers to an antigen binding domain of an antibody, i.e. the part that comprises the area which binds to and is complementary to part or all of an antigen. Accordingly, in particular aspects, an antigen binding domain herein is an antigen binding domain of an antibody. Such an antigen binding domain may be provided by an antibody fragment, for example by a Fab molecule, a single-chain antibody molecule or single-domain antibodies, such as a VHH domain.

The term “variable region” or “variable domain” refers to the domain of an antibody heavy or light chain that is involved in binding the antibody to the antigen. The term includes VHH domains. The variable domains of the heavy chain and light chain (VH and VL, respectively) of a native antibody generally have similar structures, with each domain comprising four conserved framework regions (FRs) and complementarity determining regions (CDRs). See, e.g., Kindt et al., Kuby Immunology, 6th ed., W.H. Freeman & Co., page 91 (2007). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993): Clarkson et al., Nature 352:624-628 (1991). As used herein in connection with variable region sequences, “Kabat numbering” refers to the numbering system set forth by Kabat et al., Sequences of Proteins of Immunological Interest, 5th Ed. Public Health Service, National Institutes of Health, Bethesda, MD (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991), referred to as “numbering according to Kabat” or “Kabat numbering” herein.

Specifically the Kabat numbering system (see pages 647-660 of Kabat, et al., Sequences of Proteins of Immunological Interest, 5th ed., Public Health Service, National Institutes of Health, Bethesda, MD (1991)) is used for the light chain constant domain CL of kappa and lambda isotype and the Kabat EU index numbering system (see pages 661-723) is used for the heavy chain constant domains (CH1, hinge, CH2 and CH3), which is herein further clarified by referring to “numbering according to Kabat EU index” or “Kabat EU index numbering” in this case.

The terms “binding site” or “antigen-binding site” as used herein refers to the site, i.e. one or more amino acid residues, of a binding molecule which provides interaction with the antigen. For example, the antigen binding site of an antigen binding domain comprises amino acid residues from the complementarity determining regions (CDRs). An antigen-binding site may be provided by, for example, one or more variable domains (also called variable regions). In single domain antibodies the antigen-binding site is provided by a single variable domain. Whereas, in a Fab fragment the antigen-binding site is provided by the VH and VL domains.

The term “hypervariable region” or “HVR”, as used herein, refers to each of the regions of an antigen binding domain which are hypervariable in sequence and which determine antigen binding specificity, for example “complementarity determining regions” (“CDRs”). Generally, variable domains comprise three CDRs. Thus antibodies comprising a VH and a VL comprise six CDRs: three in the VH (HCDR1, HCDR2, HCDR3), and three in the VL (LCDR1, LCDR2, LCDR3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementarity determining regions (CDRs). The FR of a variable domain generally consists of four FR domains: FR1, FR2, FR3, and FR4. Accordingly, the HVR and FR sequences generally appear in the following order in VH and VHH domains (or VL): FR1-HCDR1 (LCDR1)-FR2-HCDR2 (LCDR2)-FR3-HCDR3 (LCDR3)-FR4. Unless otherwise indicated, CDR residues and other residues in the variable domain (e.g., FR residues) are numbered herein according to Kabat et al., supra.

The term “immunoglobulin molecule” herein refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g. γ1 (IgG₁), γ2 (IgG2), γ3 (IgG3), γ4 (IgG₄), α1 (IgA1) and α2 (IgA2). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG2, IgG3, IgG₄, IgA1, and IgA2. The heavy chain constant domains that correspond to the different classes of immunoglobulins are called α, δ, ε, γ, and μ, respectively.

A “Fab molecule” or “Fab” or “Fab fragment” refers to a protein consisting of the VH and CH1 domain of the heavy chain (the “Fab heavy chain”) and the VL and CL domain of the light chain (the “Fab light chain”) of an immunoglobulin.

A “cross-Fab molecule” or “cross-Fab” or “crossover Fab molecule” refers to a Fab molecule, wherein either the variable regions or the constant regions of the heavy and light chain are exchanged. Cross-Fab engineering enables two different chain compositions of a cross-Fab molecules. On the one hand, the variable regions of the Fab heavy and light chain are exchanged, i.e. the cross-Fab molecule comprises a peptide chain composed of the light chain variable region (VL) and the heavy chain constant region (CH1), wherein the CH1 may be fused to an Fc domain, and a peptide chain composed of the heavy chain variable region (VH) and the light chain constant region (CL). On the other hand, when the constant regions of the Fab heavy and light chain are exchanged, the cross-Fab molecule comprises a peptide chain composed of the heavy chain variable region (VH) and the light chain constant region (CL), wherein the CL may be fused to an Fc domain, and a peptide chain composed of the light chain variable region (VL) and the heavy chain constant region (CH1).

The term “conventional Fab molecule” refers to a Fab molecule composed of a Fab heavy chain comprising a VH and CH1 domain and a Fab light chain comprising a VL and CL domain.

The term “multispecific” means that a binding molecule (e.g. an antibody) is able to specifically bind to at least two distinct antigens. A multispecific binding molecule (e.g. antibody) can be, for example, a bispecific binding molecule. Typically, a bispecific binding molecule comprises two antigen binding sites, each of which is specific for a different antigens. In certain aspects, the multispecific (e.g. bispecific) binding molecule is capable of simultaneously binding two antigens, particularly two antigens expressed on the same cell, on neighbouring cells, or cells in the same tissue.

The term “valent” as used herein denotes the presence of a specified number of antigen binding sites in a binding molecule. As such, the term “monovalent binding to an antigen” denotes the presence of one (and not more than one) antigen binding site specific for the antigen in the binding molecule.

As used herein, the term “antigen” refers to a molecule, such as a protein, to which an antigen binding molecule binds. Useful antigens can be found, for example, on the surfaces of tumor cells, on the surfaces of tumor stroma cells, on the surfaces of virus-infected cells, on the surfaces of other diseased cells, on the surface of immune cells, free in blood serum, and/or in the extracellular matrix (ECM). In particular aspects, the antigen is a human protein.

As used herein, the term “epitope” refers to a site on an antigen to which an antigen-binding site binds. Epitopes can be formed from a contiguous stretch of amino acids or a conformational configuration made up of different regions of noncontiguous amino acids. Epitopes often include chemically active surface groupings of antigens such as amino acids, glycan side chains, phosphoryl, or sulfonyl, and may have specific three dimensional structural characteristics, and/or specific charge characteristics. Two distinct antigen-binding domains capable of binding the same antigen may bind different epitopes of said antigen. Such distinct antigen-binding domains are said to not compete for binding if both antigen-binding domains can simultaneously bind to the antigen. In this case two distinct antigen binding domains are non-competing. Alternatively, antigen-binding domains may compete for binding, i.e. show competitive binding. This may be due to partially overlapping epitopes or simultaneous binding of the two antigen binding domains is hampered due to steric hindrance. Assays to determine whether antigen-binding domains show non-competitive or competitive binding are well known in the art, for example competitive binding analysis using ELISA, RIA, surface plasmon resonance, flow cytometry or any other quantitative or qualitative antibody-binding assay available in the art.

A “target antigen” as used herein refers to an antigen presented on the surface of a target cell, for example an antigen on a cell in a tumor, such as a cancer cell or a cell of the tumor stroma, or an antigen on a T cell.

A “tumor-associated antigen” as used herein refers to any antigen presented on the surface of a cell in a tumor such as a cancer cell or a cell of the tumor stroma. Particular tumor-associated antigens are CEA, FAP, Her2, Her3 or EGFR.

The term “Fibroblast activation protein (FAP)”, also known as Prolyl endopeptidase FAP or Seprase (EC 3.4.21), refers to any native FAP from any vertebrate source, including mammals such as primates (e.g. humans) non-human primates (e.g. cynomolgus monkeys) and rodents (e.g. mice and rats), unless otherwise indicated. FAP is expressed on Cancer-associated fibroblasts (CAFs) in the tumor stroma. The term encompasses “full-length,” unprocessed FAP as well as any form of FAP which results from processing in the cell. The term also encompasses naturally occurring variants of FAP, e.g., splice variants or allelic variants. In one embodiment, the antigen binding molecule of the invention is capable of specific binding to human, mouse and/or cynomolgus FAP. The amino acid sequence of human FAP is shown in UniProt (www.uniprot.org) accession no. Q12884 (version 149), or NCBI (www.ncbi.nlm.nih.gov/) RefSeq NP_004451.2. The extracellular domain (ECD) of human FAP extends from amino acid position 26 to 760. The amino acid sequence of mouse FAP is shown in UniProt accession no. P97321 (version 126), or NCBI RefSeq NP_032012.1. The extracellular domain (ECD) of mouse FAP extends from amino acid position 26 to 761. Preferably, an anti-FAP binding molecule binds to the extracellular domain of FAP. Exemplary anti-FAP binding molecules are described in International Patent Application No. WO 2012/020006 A2 and WO 2020/070041 A1.

The term “Carcinoembroynic antigen (CEA)”, also known as Carcinoembryonic antigen-related cell adhesion molecule 5 (CEACAM5), refers to any native CEA from any vertebrate source, including mammals such as primates (e.g. humans) non-human primates (e.g. cynomolgus monkeys) and rodents (e.g. mice and rats), unless otherwise indicated. The amino acid sequence of human CEA is shown in UniProt accession no. P06731 (version 151). CEA has long been identified as a tumor-associated antigen (Gold and Freedman, J Exp Med., 121:439-462, 1965; Berinstein N. L., J Clin Oncol., 20:2197-2207, 2002). Originally classified as a protein expressed only in fetal tissue, CEA has now been identified in several normal adult tissues. These tissues are primarily epithelial in origin, including cells of the gastrointestinal, respiratory, and urogential tracts, and cells of colon, cervix, sweat glands, and prostate (Nap et al., Tumour Biol., 9 (2-3): 145-53, 1988: Nap et al., Cancer Res., 52 (8): 2329-23339, 1992). Tumors of epithelial origin, as well as their metastases, contain CEA as a tumor associated antigen. While the presence of CEA itself does not indicate transformation to a cancerous cell, the distribution of CEA is indicative. In normal tissue, CEA is generally expressed on the apical surface of the cell (Hammarström S., Semin Cancer Biol. 9 (2): 67-81 (1999)), making it inaccessible to antibody in the blood stream. In contrast to normal tissue, CEA tends to be expressed over the entire surface of cancerous cells (Hammarström S., Semin Cancer Biol. 9 (2): 67-81 (1999)). This change of expression pattern makes CEA accessible to antibody binding in cancerous cells. In addition, CEA expression increases in cancerous cells. Furthermore, increased CEA expression promotes increased intercellular adhesions, which may lead to metastasis (Marshall J., Semin Oncol., 30 (a Suppl. 8): 30-6, 2003). The prevalence of CEA expression in various tumor entities is generally very high. In concordance with published data, own analyses performed in tissue samples confirmed its high prevalence, with approximately 95% in colorectal carcinoma (CRC), 90% in pancreatic cancer, 80% in gastric cancer, 60% in non-small cell lung cancer (NSCLC, where it is co-expressed with HER3), and 40% in breast cancer: low expression was found in small cell lung cancer and glioblastoma.

“HER2” (also known as erbB-2 or CD340) refers to any native HER2 from any vertebrate source, including mammals such as primates (e.g. humans), non-human primates (e.g. cynomolgus monkeys) and rodents (e.g. mice and rats), unless otherwise indicated. The term encompasses “full-length,” unprocessed HER2 as well as any form of HER2 that results from processing in the cell. The term also encompasses naturally occurring variants of HER2, e.g., splice variants or allelic variants. In one aspect, HER2 is human HER2. The amino acid sequence of human HER2 is shown in UniProt (www.uniprot.org) entry no. Q9UK79 (version 95).

“Epidermal Growth Factor Receptor (EGFR)”, also named Proto-oncogene c-ErbB-1 or Receptor tyrosine-protein kinase erbB-1, refers to any native EGFR from any vertebrate source, including mammals such as primates (e.g. humans) non-human primates (e.g. cynomolgus monkeys) and rodents (e.g. mice and rats), unless otherwise indicated. The amino acid sequence of human EGFR is shown in UniProt accession no. P00533.

A “T cell antigen” as used herein refers to any antigen present on the surface of a T lymphocyte. The term “PD-1”, also known as CD279, PD1 or programmed cell death protein 1, refers to any native PD-1 from any vertebrate source, including mammals such as primates (e.g. humans) non-human primates (e.g. cynomolgus monkeys) and rodents (e.g. mice and rats), in particular to the human protein PD-1 with the amino acid sequence as shown in UniProt (www.uniprot.org) accession no. Q15116.

The term “interferon gamma” or “IFNγ” or “IFN-γ” as used herein, refers to any native IFNγ from any vertebrate source, including mammals such as primates (e.g. humans) and rodents (e.g., mice and rats), unless otherwise indicated. The term encompasses unprocessed IFNγ as well as any form of IFNγ that results from processing in the cell. The term also encompasses naturally occurring variants of IFNγ, e.g. splice variants or allelic variants.

The term “interleukin-2” or “IL-2” as used herein, refers to any native IL-2 from any vertebrate source, including mammals such as primates (e.g. humans) and rodents (e.g., mice and rats), unless otherwise indicated. The term encompasses unprocessed IL-2 as well as any form of IL-2 that results from processing in the cell. The term also encompasses naturally occurring variants of IL-2, e.g. splice variants or allelic variants.

Cytokine receptors are cell-surface glycoproteins that specifically bind cytokines and transduce their signals. Generally, cytokine receptors function as oligomeric complexes consisting of typically two to four receptor chains, also termed subunits, that may be the same or different. Thus, the term “cytokine receptor complex” refers to cytokine receptors composed of at least two subunits.

The IFNγ receptor complex comprises the IFNγR1 subunit and the IFNγR2 subunit. The term “Interferon gamma receptor 1” or “IFNγR1”, also referred to as CD119 (Cluster of differentiation 119) or Interferon gamma receptor α-chain (IFNγRa), refers to any native IFNγR1 from any vertebrate source, including mammals such as primates (e.g. humans) and rodents (e.g., mice and rats), unless otherwise indicated. The term encompasses “full-length”, unprocessed IFNγR1 as well as any form of IFNγR1 that results from processing in the cell. The term also encompasses naturally occurring variants of IFNγR1, e.g. splice variants or allelic variants. In certain embodiments IFNγR1 is human IFNγR1.

The term “Interferon gamma receptor 2” or “IFNγR2”, also referred to as Interferon gamma receptor β-chain (IFNγRB), refers to any native IFNγR2 from any vertebrate source, including mammals such as primates (e.g. humans) and rodents (e.g., mice and rats), unless otherwise indicated. The term encompasses “full-length”, unprocessed IFNγR2 as well as any form of IFNγR2 that results from processing in the cell. The term also encompasses naturally occurring variants of IFNγR2, e.g. splice variants or allelic variants. In certain embodiments IFNγR2 is human IFNγR2.

The term “IL-2Rα” or “α-subunit of the IL-2 receptor” also known as CD25, as used herein, refers to any native IL-2Rα from any vertebrate source, including mammals such as primates (e.g. humans) and rodents (e.g., mice and rats), unless otherwise indicated. The term encompasses “full-length”, unprocessed IL-2Rα as well as any form of IL-2Rα that results from processing in the cell. The term also encompasses naturally occurring variants of IL-2Rα, e.g. splice variants or allelic variants. In certain embodiments IL-2Rα is human IL-2Rα.

The term “IL-2Rβ” or “β-subunit of the IL-2 receptor”, also known as CD122 or p70, refers to any native IL-2Rβ from any vertebrate source, including mammals such as primates (e.g. humans) and rodents (e.g., mice and rats), unless otherwise indicated. The term encompasses “full-length”, unprocessed IL-2Rβ as well as any form of IL-2Rβ that results from processing in the cell. The term also encompasses naturally occurring variants of IL-2Rβ, e.g. splice variants or allelic variants. In certain embodiments IL-2Rβ is human IL-2Rβ.

The term “IL-2Rγ” or “γ-subunit of the IL-2 receptor”, also known as common cytokine receptor γ-subunit, common γ-chain, γc, or CD132, refers to any native IL-2Rγ from any vertebrate source, including mammals such as primates (e.g. humans) and rodents (e.g., mice and rats), unless otherwise indicated. The term encompasses “full-length”, unprocessed IL-2Rγ as well as any form of IL-2Rγ that results from processing in the cell. The term also encompasses naturally occurring variants of IL-2Rγ, e.g. splice variants or allelic variants. In certain embodiments IL-2Rγ is human IL-2Rγ.

Different associations of the individual IL-2R subunits IL-2Rα, IL-2Rβ and IL-2Rγ, can produce three IL-2R forms that differ in their affinity to IL-2. The high-affinity IL-2R refers to the heterotrimeric form of the IL-2R, consisting of IL-2Rα, IL-2Rβ and IL-2Rγ. The intermediate-affinity IL-2R refers to the heterodimeric form of the IL-2R, consisting of IL-2Rβ and IL-2Rγ. Whereas, the low affinity IL-2R refers to the monomeric form of the IL-2R, consisting solely of IL-2Rα (for a review see e.g. Olejniczak and Kasprzak, Med Sci Monit 14, RA179-189 (2008)).

The term “IL-2 receptor complex” as used herein refers to the high-affinity IL-2 receptor or the intermediate-affinity IL-2 receptor.

The term “Fc domain” or “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (Lys447), of the Fc region may or may not be present. In one aspect, a heavy chain including an Fc region (subunit) as specified herein, comprised in a binding molecule according to the invention, comprises an additional C-terminal lysine (K447, numbering according to Kabat EU index). In one aspect, a heavy chain including an Fc region (subunit) as specified herein, comprised in a binding molecule according to the invention, comprises a C-terminal glycine residue (G446, numbering according to Kabat EU index) and does not comprise a C-terminal lysine (Lys447). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein preferably includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which may be non-identical in the sense that further components fused to each of the subunits (e.g. antigen binding domains) are not the same. In one aspect, the modification promoting the association of the first and the second subunit of the Fc domain comprises an amino acid mutation in the Fc domain, specifically an amino acid substitution. In particular aspects, the modification promoting the association of the first and the second subunit of the Fc domain comprises a separate amino acid mutation, specifically an amino acid substitution, in each of the two subunits of the Fc domain.

One heterodimerization approach known in the art is the so-called “knobs-into-holes” technology, which is described in detail providing several examples in e.g. WO 96/027011, Ridgway, J. B., et al., Protein Eng. 9 (1996) 617-621: Merchant, A. M., et al., Nat. Biotechnol. 16 (1998) 677-681; and WO98/050431. In the “knobs-into-holes” technology, within the interface formed between two CH3 domains in the tertiary structure of the antibody, particular amino acids on each CH3 domain are engineered to produce a protuberance (“knob”) in one of the CH3 domains and a cavity (“hole”) in the other one of the CH3 domains, respectively. In the tertiary structure of the multispecific antibody the introduced protuberance in the one CH3 domain is positionable in the introduced cavity in the other CH3 domain.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B-cell receptor), and B-cell activation.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

Antibody-dependent cell-mediated cytotoxicity (ADCC) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example, the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT publication no. WO 2006/082515 or PCT publication no. WO 2012/130831).

“Reduced binding”, for example reduced binding to an Fc receptor, refers to a decrease in affinity for the respective interaction, as measured for example by SPR. For clarity, the term includes also reduction of the affinity to zero (or below the detection limit of the analytic method), i.e. complete abolishment of the interaction. Conversely, “increased binding” refers to an increase in binding affinity for the respective interaction.

“Affinity.” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., an antibody and an antigen). The affinity of a molecule X for its partner Y can generally be represented by the dissociation constant (KD). Affinity can be measured by well-established methods known in the art, including those described herein. A preferred method for measuring affinity is Surface Plasmon Resonance (SPR).

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor, or increased association with another peptide. Amino acid sequence deletions and insertions include amino- and/or carboxy-terminal deletions and insertions of amino acids. Preferred amino acid mutations are amino acid substitutions. For the purpose of altering e.g. the binding characteristics of an Fc region, nonconservative amino acid substitutions, i.e. replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation. For example, a substitution from proline at position 329 of the Fc domain to glycine can be indicated as 329G, G329, G329, P329G, or Pro329Gly.

By “fused” is meant that the components (e.g. a Fab molecule and an Fc domain subunit) are linked by peptide bonds, either directly or via one or more peptide linkers.

The term “linker” or “peptide linker” refers to a peptide comprising one or more amino acids, typically about 2 to 30 amino acids. Peptide linkers are known in the art or are described herein. Suitable, non-immunogenic linker peptides are, for example, (G4S) n peptide linkers, wherein G-glycine, S=serine, and wherein “n” is generally a number between 1 and 10. Additionally, linkers may comprise (a portion of) an immunoglobulin hinge region. Particularly where a Fab molecule is fused to the N-terminus of an Fc domain subunit, it may be fused via an immunoglobulin hinge region or a portion thereof.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, % amino acid sequence identity values are generated using the ggsearch program of the FASTA package version 36.3.8c or later with a BLOSUM50 comparison matrix. The FASTA program package was authored by W. R. Pearson and D. J. Lipman (“Improved Tools for Biological Sequence Analysis”, PNAS 85 (1988) 2444-2448), W. R. Pearson (“Effective protein sequence comparison” Meth. Enzymol. 266 (1996) 227-25 258), and Pearson et. al. (Genomics 46 (1997) 24-36) and is publicly available from www.fasta.bioch.virginia.edu/fasta_www2/fasta_down.shtml or www.ebi.ac.uk/Tools/sss/fasta.

Alternatively, a public server accessible at fasta.bioch.virginia.edu/fasta_www2/index.cgi can be used to compare the sequences, using the ggsearch (global protein: protein) program and default options (BLOSUM50: open: −10: ext: −2: Ktup=2) to ensure a global, rather than local, alignment is performed. Percent amino acid identity is given in the output alignment header.

### Cytokine Mimetics

The present inventors have found that a pair of antigen binding domains capable of binding different cytokine receptor subunits of a cytokine receptor complex, hereinafter referred to as cytokine receptor-binding domains, can act as cytokine agonists, i.e. mimic a naturally occurring cytokine. Cytokine agonists are also termed cytokine mimetics. This has been shown by combining a pair of cytokine receptor-binding domains capable of binding different cytokine receptor subunits in a single antigen binding molecule. The antibody format of these molecules may provide advantageous properties compared to natural or recombinant cytokines. To achieve conditional activation of a cytokine receptor, the pair of cytokine receptor-binding domains were split in two distinct antigen binding molecules, i.e. split molecules or split antibodies. These antigen binding molecules further comprise an antigen binding domain capable of binding a target antigen, hereinafter referred to as target-binding domain. By target-dependent assembly of the pair of cytokine receptor-binding domains, the molecules can act as cytokine mimetics. Using a target-dependent approach further allows to direct the agonistic activity to a site of interest, such as cells or tissues expressing the target. The target-binding domains of the molecules are capable of binding the same target antigen simultaneously, i.e. they bind different epitopes of said antigen and are non-competing. Such a pair of target-binding domains are also referred to as biparatopic target-binding domains. The assembly of the cytokine receptor-binding domains by the biparatopic target-binding domains allows to target the cytokine receptor-binding domains specifically to a site of interest, i.e. a cell or environment where the target antigen is expressed, to act as cytokine mimetics. The pair of split antigen binding molecules are thus a biparatopic pair of antigen binding molecules. Finally, the pair of cytokine receptor-binding domains and target-binding domains were incorporated into a single molecule, hereinafter referred to as the all-in-one format. Target-independent assembly of the cytokine receptor-binding domains, i.e. assembly in absence of the target antigen, is prevented by distancing the cytokine receptor-binding domains from each other. While in presence of the target antigen, biparatopic assembly of the cytokine receptor-binding domains results in activation of the cytokine receptor.

### All-In-One Format

The all-in-one format provides an antigen binding molecule comprising i) a first target-binding domain, ii) a second target-binding domain, iii) a first cytokine receptor-binding domain, iv) a second cytokine receptor-binding domain, and v) a Fc domain, herein the first target-binding domain is capable of binding a first epitope on the target antigen and second target-binding domain is capable of binding a second epitope on the target antigen, wherein the first and second target-binding domains do not compete for binding on the target antigen, and wherein the first cytokine receptor-binding domain is capable of binding a first cytokine receptor subunit and the second cytokine receptor-binding domain is capable of binding a second cytokine receptor subunit.

### Cytokine Receptor-Binding Domain

According to the invention, the all-in-one antigen binding molecule comprises a first cytokine receptor-binding domain and a second cytokine receptor-binding domain. The cytokine receptor-binding domains comprised in the antigen binding molecules bind different cytokine receptor subunits. Each cytokine receptor-binding domain of antigen binding molecule binds a distinct cytokine receptor subunit of a cytokine receptor complex. Thus, the all-in-one antigen binding molecule comprises a first cytokine receptor-binding domain capable of binding a first cytokine receptor subunit and a second cytokine receptor-binding domain capable of binding a second cytokine receptor subunit. The first and the second cytokine receptor subunit are subunits of a cytokine receptor complex.

The cytokine receptor complex may be IFNγ receptor complex, IL-2 receptor complex, IL-7 receptor complex, IL-12 receptor complex or IL-18 receptor complex. Thus, the first and the second cytokine receptor-binding domain may bind different subunits of the IFNγ receptor complex, IL-2 receptor complex, IL-7 receptor complex, IL-12 receptor complex or IL-18 receptor complex. In one embodiment, the first and second cytokine receptor-binding domains bind subunits of the IFNγ receptor complex. Thus, in one embodiment the first cytokine receptor-binding domain is capable of binding IFNγR1 and the second cytokine receptor-binding domain is capable of binding IFNγR2. Alternatively, the first cytokine receptor-binding domain is capable of binding IFNγR2 and the second cytokine receptor-binding domain is capable of binding IFNγR1. In a further embodiment, the first and second cytokine receptor-binding domains bind subunits of the IL-2 receptor complex. Thus, in one embodiment the first cytokine receptor-binding domain is capable of binding IL-2Rβ and the second cytokine receptor-binding domain is capable of binding IL-2Rγ. In one embodiment, the first cytokine receptor-binding domain is capable of binding IL-2Rγ and the second cytokine receptor-binding domain is capable of binding IL-2Rβ. In another embodiment, the first and second cytokine receptor-binding domains bind subunits of the IL-7 receptor complex. Thus, in one embodiment the first cytokine receptor-binding domain is capable of binding IL-7Ra and the second cytokine receptor-binding domain is capable of binding IL-2Rγ. In one embodiment, the first cytokine receptor-binding domain is capable of binding IL-2Rγ and the second cytokine receptor-binding domain is capable of binding IL-7Ra. In a further embodiment, the first and second cytokine receptor-binding domains bind subunits of the IL-12 receptor complex. Thus, in one embodiment the first cytokine receptor-binding domain is capable of binding IL-12Rβ1 and the second cytokine receptor-binding domain is capable of binding IL-12Rβ2. In one embodiment, the first cytokine receptor-binding domain is capable of binding IL-12Rβ2 and the second cytokine receptor-binding domain is capable of binding IL-12Rβ1.

The first and second cytokine receptor-binding domains may be antibody fragments. In one embodiment, the first and second cytokine receptor-binding domains are single-domain antibodies. In a particular embodiment, the first and second cytokine receptor-binding domains are VHH domains.

In one aspect, the first cytokine receptor-binding domain comprises an amino acid sequence selected from SEQ ID NO: 1, SEQ ID NO: 2, SEQ ID NO: 3, SEQ ID NO: 4 and SEQ ID NO: 5 and the second cytokine receptor-binding domain comprises an amino acid sequence selected from SEQ ID NO: 6, SEQ ID NO: 7, SEQ ID NO: 8, SEQ ID NO: 9 and SEQ ID NO: 10. In one aspect, the first cytokine receptor-binding domain comprises an amino acid sequence selected from SEQ ID NO: 6, SEQ ID NO: 7, SEQ ID NO: 8, SEQ ID NO: 9 and SEQ ID NO: 10, and the second cytokine receptor-binding domain comprises an amino acid sequence selected from SEQ ID NO: 1, SEQ ID NO: 2, SEQ ID NO: 3, SEQ ID NO: 4 and SEQ ID NO: 5. In one aspect, the first cytokine receptor-binding domain comprises the sequence of SEQ ID NO: 3 and the second cytokine receptor-binding domain comprises the sequence of SEQ ID NO: 7. In one aspect, the first cytokine receptor-binding domain comprises the sequence of SEQ ID NO: 7 and the second cytokine receptor-binding domain comprises the sequence of SEQ ID NO: 3.

In one aspect, the first cytokine receptor-binding domain comprises an amino acid sequence selected from SEQ ID NO: 60, SEQ ID NO: 61, SEQ ID NO: 62, SEQ ID NO: 63, SEQ ID NO: 64, and the second cytokine receptor-binding domain comprises an amino acid sequence selected from SEQ ID NO: 65, SEQ ID NO: 66, SEQ ID NO: 67, SEQ ID NO: 68, SEQ ID NO: 69. In one aspect, the first cytokine receptor-binding domain comprises an amino acid sequence selected from SEQ ID NO: 65, SEQ ID NO: 66, SEQ ID NO: 67, SEQ ID NO: 68, SEQ ID NO: 69, and the second cytokine receptor-binding domain comprises an amino acid sequence selected from SEQ ID NO: 60, SEQ ID NO: 61, SEQ ID NO: 62, SEQ ID NO: 63, SEQ ID NO: 64. In one aspect, the first cytokine receptor-binding domain comprises the sequence of SEQ ID NO: 64 and the second cytokine receptor-binding domain comprises the sequence of SEQ ID NO: 66. In one aspect, the first cytokine receptor-binding domain comprises the sequence of SEQ ID NO: 66 and the second cytokine receptor-binding domain comprises the sequence of SEQ ID NO: 64.

### Target Antigen-Binding Domain

According to the invention, the antigen binding molecule comprises a first target-binding domain and a second target-binding domain. Both the first and the second target-binding domain bind the same target, i.e. the same antigen. Yet, the first and the second target-binding domains bind distinct epitopes of said target. The first target-binding domain is capable of binding a first epitope on the target antigen and the second target-binding domain is capable of binding a second epitope on the target antigen. The first and the second target-binding domain are capable of binding the target simultaneously, i.e. the target-binding domains are non-competing domains. The first target-binding domain and the second target-binding domain do not compete for binding on the target antigen.

The target-binding domains of the antigen binding molecule may be antibody fragments. In one embodiment, the first target-binding domain is an antibody fragment and the second target-binding domain is an antibody fragment. The first and/or second target-binding domains may be a Fv, Fab, scFv, scFab molecule or a single domain antibody. In one embodiment, the target binding domain is a Fv, Fab, scFv, scFab molecule or a single domain antibody, and the target-binding domain is a Fv, Fab, scFv, scFab molecule or a single domain antibody.

In one embodiment, the first and second target-binding domains are Fab molecules. In one embodiment, the first target-binding domain is a Fab molecule and the second target-binding domain is a Fab molecule. In one embodiment, the first target-binding domain comprises a heavy chain variable domain (VH₁), a light chain variable domain (VL₁), a heavy chain constant domain (CH1₁) and a light chain constant domain (CL₁). In one embodiment, the second target-binding domain comprises a heavy chain variable domain (VH₂), a light chain variable domain (VL₂), a heavy chain constant domain (CH1₂) and a light chain constant domain (CL₂).

The target-binding domain may be a cross-Fab. In one embodiment, the first target-binding domain is a cross-Fab. In another embodiment, the second target-binding domain is a cross-Fab. In one embodiment, the first target-binding domain is a Fab molecule, wherein the Fab molecule is a cross-Fab, and the second target-binding domain is a Fab molecule, wherein the Fab molecule is not a cross-Fab. In one embodiment, the first target-binding domain is Fab molecule, wherein the Fab molecule is a conventional Fab molecule, and the second target-binding domain is a Fab molecule, wherein the Fab molecule is a cross-Fab.

In one aspect, a Fab molecule comprises charged modifications. To reduce mispairing of heavy and light chains from the different Fab molecules and thus to increase the purity and yield of the desired antigen binding molecule, Fab molecules can contain different charged amino acid substitutions (so-called “charged modifications”). These modifications are introduced in the cross-Fab CH1 and CL domains or in the conventional Fab CH1 and CL domains. In a particular aspect, the Fab molecule is one, wherein in the CL domain the amino acid at position 123 (EU numbering) has been replaced by arginine (R) and/or wherein the amino acid at position 124 (EU numbering) has been substituted by lysine (K) and wherein in the CH1 domain the amino acids at position 147 (EU numbering) and/or at position 213 (EU numbering) have been substituted by glutamic acid (E). Preferably, the charge modifications are made in a conventional Fab molecule.

The charged modifications may be introduced in the CH1 and CL domains of either the Fab molecule of the first target-binding domain or the Fab molecule of the second target-binding domain. In one aspect, the first target-binding domain comprises charge modifications. In one aspect, the second target-binding domain comprises charge modifications. In another aspect, the first target-binding domain comprises charge modifications and the second target-binding domain comprises a cross-Fab. In a further aspect, the first target-binding domain comprises a cross-Fab and the second target-binding domain comprises charge modifications.

Both the first and the second target-binding domains specifically bind the same target antigen. The target antigen may be a tumor-associated antigen or a T cell antigen. The target antigen may be a tumor-associated antigen such as CEA, FAP, Her2, Her3 or EGFR. Thus, in one aspect the first target-binding domain is capable of binding a first epitope of CEA and the second target-binding domain is capable of binding a second epitope of CEA. In one aspect the first target-binding domain is capable of binding a first epitope of FAP and the second target-binding domain is capable of binding a second epitope of FAP. In one aspect the first target-binding domain is capable of binding a first epitope of Her2 and the second target-binding domain is capable of binding a second epitope of Her2. In one aspect the first target-binding domain is capable of binding a first epitope of Her3 and the second target-binding domain is capable of binding a second epitope of Her3. In one aspect the first target-binding domain is capable of binding a first epitope of EGFR and the second target-binding domain is capable of binding a second epitope of EGFR.

In one aspect, the first target-binding domain comprises a VH₁ of SEQ ID NO: 20 and a VL₁ of SEQ ID NO: 21 and the second target-binding domain comprises a VH₂ of SEQ ID NO: 22 and a VL₂ of SEQ ID NO: 23. In one aspect, the first target-binding domain comprises a VH₁ of SEQ ID NO: 22 and a VL₁ of SEQ ID NO: 23 and the second target-binding domains comprises a VH₂ of SEQ ID NO: 20 and a VL₂ of SEQ ID NO: 21.

The target antigen may be a T cell antigen such as PD-1 or LAG-3. The target antigen may be human PD-1 or human LAG-3. Thus, in one aspect the first target-binding domain is capable of binding a first epitope of PD-1 and the second target-binding domain is capable of binding a second epitope of PD-1. In one aspect the first target-binding domain is capable of binding a first epitope of LAG-3 and the second target-binding domain is capable of binding a second epitope of LAG-3. In one aspect, the first target-binding domain comprises a VH₁ of SEQ ID NO: 80 and a VL₁ of SEQ ID NO: 81 and the second target-binding domain comprises a VH₂ of SEQ ID NO: 82 and a VL₂ of SEQ ID NO: 83. In one aspect, the first target-binding domain comprises a VH₁ of SEQ ID NO: 82 and a VL₁ of SEQ ID NO: 83 and the second target-binding domains comprises a VH₂ of SEQ ID NO: 80 and a VL₂ of SEQ ID NO: 81. In one aspect, the first target-binding domain comprises a VH₁ of SEQ ID NO: 82 and a VL₁ of SEQ ID NO: 83 and the second target-binding domain comprises a VH₂ of SEQ ID NO: 140 and a VL₂ of SEQ ID NO: 141. In one aspect, the first target-binding domain comprises a VH₁ of SEQ ID NO: 140 and a VL₁ of SEQ ID NO: 141 and the second target-binding domain comprises a VH₂ of SEQ ID NO: 82 and a VL₂ of SEQ ID NO: 83.

### Fc Domain

According to the invention, the antigen binding molecule comprise an Fc domain.

The Fc domain of the binding molecule consists of a pair of polypeptide chains, a first Fc domain subunit and a second Fc domain subunit. The first and second Fc domain subunits may comprise heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other.

In one aspects, the antigen binding molecule comprises a Fc domain composed of a first and a second Fc domain subunit. In a particular aspects, the antigen binding molecule comprises not more than one Fc domain.

In one aspect, the Fc domain of the antigen binding molecule is an IgG Fc domain. In particular aspects, the Fc domain is an IgG Fc domain. In other aspects, the Fc domain is an IgG₄ Fc domain.

In a more specific aspect, the Fc domain is an IgG₄ Fc domain comprising an amino acid substitution at position S228 (Kabat EU index numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In further aspects, the Fc domain is a human Fc domain. In particular aspects, the Fc domain is a human IgG₁ Fc domain.

In one aspect, the Fc domain comprises a modification promoting the association of the first and the second subunit of the Fc domain. Fc domain modifications promoting heterodimerization are further described hereinbelow.

In one aspect, the Fc domain comprises one or more amino acid substitution that reduces binding to an Fc receptor and/or effector function. Fc domain modifications reducing Fc receptor binding and/or effector function are further described hereinbelow.

### Fc Domain Modifications Promoting Heterodimerization

The antigen binding molecule according to the invention comprises two target-binding domains and two cytokine receptor-binding domains, which may be fused to the first or the second Fc domain subunit, thus the two Fc domain subunits are typically comprised in two non-identical polypeptide chains. Recombinant co-expression of these polypeptides and subsequent dimerization leads to several possible combinations of the two polypeptides. To improve the yield and purity of the antigen binding molecule in recombinant production, it will thus be advantageous to introduce in the Fc domain of the antigen binding molecule a modification promoting the association of the desired polypeptides.

Accordingly, in particular aspects, the Fc domain of the antigen binding molecule according to the invention comprises a modification promoting the association of the first and the second subunit of the Fc domain. The site of most extensive protein-protein interaction between the two subunits of a human IgG Fc domain is in the CH3 domain of the Fc domain. Thus, in one aspect, said modification is in the CH3 domain of the Fc domain.

There exist several approaches for modifications in the CH3 domain of the Fc domain in order to enforce heterodimerization, which are well described e.g. in WO 96/27011, WO 98/050431, EP 1870459, WO 2007/110205, WO 2007/147901, WO 2009/089004, WO 2010/129304, WO 2011/90754, WO 2011/143545, WO 2012058768, WO 2013157954, WO 2013096291. Typically, in all such approaches the CH3 domain of the first subunit of the Fc domain and the CH3 domain of the second subunit of the Fc domain are both engineered in a complementary manner so that each CH3 domain (or the heavy chain comprising it) can no longer homodimerize with itself but is forced to heterodimerize with the complementarily engineered other CH3 domain (so that the first and second CH3 domain heterodimerize and no homdimers between the two first or the two second CH3 domains are formed).

In specific aspects, said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan).

Compensatory cavities of identical or similar size to the protuberances are created in the interface of the second polypeptide by replacing large amino acid side chains with smaller ones (e.g. alanine or threonine).

Accordingly, in preferred aspects, in the CH3 domain of the first subunit of the Fc domain of the antigen binding molecule an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine(S), threonine (T), and valine (V). The protuberance and cavity can be made by altering the nucleic acid encoding the polypeptides, e.g. by site-specific mutagenesis, or by peptide synthesis.

In specific aspects, in (the CH3 domain of) the first subunit of the Fc domain (the “knobs” subunit) the threonine residue at position 366 is replaced with a tryptophan residue (T366W), and in (the CH3 domain of) the second subunit of the Fc domain (the “hole” subunit) the tyrosine residue at position 407 is replaced with a valine residue (Y407V). In one aspect, in the second subunit of the Fc domain additionally the threonine residue at position 366 is replaced with a serine residue (T366S) and the leucine residue at position 368 is replaced with an alanine residue (L368A) (numberings according to Kabat EU index).

In yet further aspects, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numberings according to Kabat

EU index). Introduction of these two cysteine residues results in formation of a disulfide bridge between the two subunits of the Fc domain, further stabilizing the dimer (Carter, J Immunol Methods 248, 7-15 (2001)).

In particular aspects, the first subunit of the Fc domain comprises the amino acid substitutions S354C and T366W, and the second subunit of the Fc domain comprises the amino acid substitutions Y349C, T366S, L368A and Y407V (numbering according to Kabat EU index).

Other techniques of CH3-modification for enforcing the heterodimerization are contemplated as alternatives according to the invention and are described e.g. in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291.

In one aspect, the heterodimerization approach described in EP 1870459, is used alternatively. This approach is based on the introduction of charged amino acids with opposite charges at specific amino acid positions in the CH3/CH3 domain interface between the two subunits of the Fc domain.

A particular aspect for the antigen binding molecule of the invention are amino acid mutations R409D: K370E in one of the two CH3 domains (of the Fc domain) and amino acid mutations D399K: E357K in the other one of the CH3 domains of the Fc domain (numbering according to Kabat EU index).

In one aspect, the antigen binding molecule of the invention comprises amino acid mutation T366W in the CH3 domain of the first subunit of the Fc domain and amino acid mutations T366S, L368A, Y407V in the CH3 domain of the second subunit of the Fc domain, and additionally amino acid mutations R409D: K370E in the CH3 domain of the first subunit of the Fc domain and amino acid mutations D399K: E357K in the CH3 domain of the second subunit of the Fc domain (numberings according to Kabat EU index).

In one aspect, the antigen binding molecule of the invention comprises amino acid mutations S354C, T366W in the CH3 domain of the first subunit of the Fc domain and amino acid mutations Y349C, T366S, L368A, Y407V in the CH3 domain of the second subunit of the Fc domain, or said antigen binding molecule comprises amino acid mutations Y349C, T366W in the CH3 domain of the first subunit of the Fc domain and amino acid mutations S354C, T366S, L368A, Y407V in the CH3 domains of the second subunit of the Fc domain and additionally amino acid mutations R409D: K370E in the CH3 domain of the first subunit of the Fc domain and amino acid mutations D399K: E357K in the CH3 domain of the second subunit of the Fc domain (all numberings according to Kabat EU index).

In one aspect, the heterodimerization approach described in WO 2013/157953 is used alternatively. In one aspect, a first CH3 domain comprises amino acid mutation T366K and a second CH3 domain comprises amino acid mutation L351D (numberings according to Kabat EU index). In further aspects, the first CH3 domain comprises further amino acid mutation L351K. In further aspects, the second CH3 domain comprises further an amino acid mutation selected from Y349E, Y349D and L368E (particularly L368E) (numberings according to Kabat EU index).

In one aspect, the heterodimerization approach described in WO 2012/058768 is used alternatively. In one aspect, a first CH3 domain comprises amino acid mutations L351Y, Y407A and a second CH3 domain comprises amino acid mutations T366A, K409F. In further aspects, the second CH3 domain comprises a further amino acid mutation at position T411, D399, S400, F405, N390, or K392, e.g. selected from a) T411N, T411R, T411Q, T411K, T411D, T411E or T411W, b) D399R, D399W, D399Y or D399K, c) S400E, S400D, S400R, or S400K, d) F4051, F405M, F405T, F405S, F405V or F405W, e) N390R, N390K or N390D, f) K392V, K392M, K392R, K392L, K392F or K392E (numberings according to Kabat EU index). In further aspects, a first CH3 domain comprises amino acid mutations L351Y, Y407A and a second CH3 domain comprises amino acid mutations T366V, K409F. In further aspects, a first CH3 domain comprises amino acid mutation Y407A and a second CH3 domain comprises amino acid mutations T366A, K409F. In further aspects, the second CH3 domain further comprises amino acid mutations K392E, T411E, D399R and S400R (numberings according to Kabat EU index).

In one aspect, the heterodimerization approach described in WO 2011/143545 is used alternatively, e.g. with the amino acid modification at a position selected from the group consisting of 368 and 409 (numbering according to Kabat EU index).

In one aspect, the heterodimerization approach described in WO 2011/090762, which also uses the knobs-into-holes technology described above, is used alternatively. In one aspect, a first CH3 domain comprises amino acid mutation T366W and a second CH3 domain comprises amino acid mutation Y407A. In one aspect, a first CH3 domain comprises amino acid mutation T366Y and a second CH3 domain comprises amino acid mutation Y407T (numberings according to Kabat EU index).

In one aspect, the antigen binding molecule or its Fc domain is of IgG₂ subclass and the heterodimerization approach described in WO 2010/129304 is used alternatively.

In an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable. In some such aspects, a first CH3 domain comprises amino acid substitution of K392 or N392 with a negatively charged amino acid (e.g. glutamic acid (E), or aspartic acid (D), particularly K392D or N392D) and a second CH3 domain comprises amino acid substitution of D399, E356, D356, or E357 with a positively charged amino acid (e.g. lysine (K) or arginine (R), particularly D399K, E356K, D356K, or E357K, and more particularly D399K and E356K). In further aspects, the first CH3 domain further comprises amino acid substitution of K409 or R409 with a negatively charged amino acid (e.g. glutamic acid (E), or aspartic acid (D), particularly K409D or R409D). In further aspects, the first CH3 domain further or alternatively comprises amino acid substitution of K439 and/or K370 with a negatively charged amino acid (e.g. glutamic acid (E), or aspartic acid (D)) (all numberings according to Kabat EU index).

In one aspect, the heterodimerization approach described in WO 2007/147901 is used alternatively. In one aspect, a first CH3 domain comprises amino acid mutations K253E, D282K, and K322D and a second CH3 domain comprises amino acid mutations D239K, E240K, and K292D (numberings according to Kabat EU index). In one aspect, the heterodimerization approach described in WO 2007/110205 can be used alternatively.

In one aspect, the first subunit of the Fc domain comprises amino acid substitutions K392D and K409D, and the second subunit of the Fc domain comprises amino acid substitutions D356K and D399K (numbering according to Kabat EU index).

Fc Domain Modifications Reducing Fc Receptor Binding and/or Effector Function

The Fc domain confers to the antigen binding molecule favorable pharmacokinetic properties, including a long serum half-life which contributes to good accumulation in the target tissue and a favorable tissue-blood distribution ratio. At the same time it may, however, lead to undesirable targeting of the binding molecule to cells expressing Fc receptors rather than to the preferred antigen-bearing cells. Moreover, the co-activation of Fc receptor signaling pathways may lead to cytokine release which may result in excessive activation of cytokine receptors and severe side effects upon systemic administration. Activation of (Fc receptor-bearing) immune cells other than T cells may even reduce efficacy of the pair of antigen binding molecules due to the potential destruction of T cells e.g. by NK cells.

Accordingly, in particular aspects, the Fc domain of the antigen binding molecule according to the invention exhibits reduced binding affinity to an Fc receptor and/or reduced effector function, as compared to a native IgG₁ Fc domain. In some such aspects, the Fc domain (or the antigen binding molecule comprising said Fc domain) exhibits less than 50%, particularly less than 20%, more particularly less than 10% and most particularly less than 5% of the binding affinity to an Fc receptor, as compared to a native IgG₁ Fc domain (or a antigen binding molecule comprising a native IgG₁ Fc domain), and/or less than 50%, particularly less than 20%, more particularly less than 10% and most particularly less than 5% of the effector function, as compared to a native IgG₁ Fc domain domain (or a antigen binding molecule comprising a native IgG₁ Fc domain). In one aspect, the Fc domain domain (or the antigen binding molecule comprising said Fc domain) does not substantially bind to an Fc receptor and/or induce effector function. In particular aspects, the Fc receptor is an Fcγ receptor. In one aspect, the Fc receptor is a human Fc receptor. In one aspect, the Fc receptor is an activating Fc receptor. In specific aspects, the Fc receptor is an activating human Fcγ receptor, more specifically human FcγRIIIa, FcγR1 or FcγRIIa, most specifically human FcγRIIIa. In one aspect, the effector function is one or more selected from the group of CDC, ADCC, ADCP, and cytokine secretion. In particular aspects, the effector function is ADCC. In one aspect, the Fc domain domain exhibits substantially similar binding affinity to neonatal Fc receptor (FcRn), as compared to a native IgG₁ Fc domain domain. Substantially similar binding to FcRn is achieved when the Fc domain (or the antigen binding molecule comprising said Fc domain) exhibits greater than about 70%, particularly greater than about 80%, more particularly greater than about 90% of the binding affinity of a native IgG₁ Fc domain (or the antigen binding molecule comprising a native IgG₁ Fc domain) to FcRn.

In certain aspects, the Fc domain is engineered to have reduced binding affinity to an Fc receptor and/or reduced effector function, as compared to a non-engineered Fc domain. In particular aspects, the Fc domain of the antigen binding molecule comprises one or more amino acid mutation that reduces the binding affinity of the Fc domain to an Fc receptor and/or effector function. Typically, the same one or more amino acid mutation is present in each of the two subunits of the Fc domain. In one aspect, the amino acid mutation reduces the binding affinity of the Fc domain to an Fc receptor. In one aspect, the amino acid mutation reduces the binding affinity of the Fc domain to an Fc receptor by at least 2-fold, at least 5-fold, or at least 10-fold. In aspects where there is more than one amino acid mutation that reduces the binding affinity of the Fc domain to the Fc receptor, the combination of these amino acid mutations may reduce the binding affinity of the Fc domain to an Fc receptor by at least 10-fold, at least 20-fold, or even at least 50-fold. In one aspect, the antigen binding molecule(s) comprising an engineered Fc domain exhibits less than 20%, particularly less than 10%, more particularly less than 5% of the binding affinity to an Fc receptor as compared to a antigen binding molecule comprising a non-engineered Fc domain. In particular aspects, the Fc receptor is an Fcγ receptor. In one aspect, the Fc receptor is a human Fc receptor. In one aspect, the Fc receptor is an activating Fc receptor. In specific aspects, the Fc receptor is an activating human Fcγ receptor, more specifically human FcγRIIIa, FcγR1 or FcγRIIa, most specifically human FcγRIIIa. Preferably, binding to each of these receptors is reduced. In one aspect, binding affinity to a complement component, specifically binding affinity to C1q, is also reduced. In one aspect, binding affinity to neonatal Fc receptor (FcRn) is not reduced.

Substantially similar binding to FcRn, i.e. preservation of the binding affinity of the Fc domain to said receptor, is achieved when the Fc domain (or the antigen binding molecule comprising said Fc domain) exhibits greater than about 70% of the binding affinity of a non-engineered form of the Fc domain (or the antigen binding molecule comprising said non-engineered form of the Fc domain) to FcRn. The Fc domain, or antigen binding molecule(s) of the invention comprising said Fc domain, may exhibit greater than about 80% and even greater than about 90% of such affinity. In certain aspects, the Fc domain of the antigen binding molecule(s) is engineered to have reduced effector function, as compared to a non-engineered Fc domain. The reduced effector function can include, but is not limited to, one or more of the following: reduced complement dependent cytotoxicity (CDC), reduced antibody dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced crosslinking of target-bound antibodies, reduced dendritic cell maturation, or reduced T cell priming. In one aspect, the reduced effector function is one or more selected from the group of reduced CDC, reduced ADCC, reduced ADCP, and reduced cytokine secretion. In particular aspects, the reduced effector function is reduced ADCC. In one aspect, the reduced ADCC is less than 20% of the ADCC induced by a non-engineered Fc domain (or a binding molecule comprising a non-engineered Fc domain).

In one aspect, the amino acid mutation that reduces the binding affinity of the Fc domain to an Fc receptor and/or effector function is an amino acid substitution. In one aspect, the Fc domain comprises an amino acid substitution at a position selected from the group of E233, L234, L235, N297, P331 and P329 (numberings according to Kabat EU index). In a more specific aspect, the Fc domain comprises an amino acid substitution at a position selected from the group of L234, L235 and P329 (numberings according to Kabat EU index). In one aspect, the Fc domain comprises the amino acid substitutions L234A and L235A (numberings according to Kabat EU index). In some such aspects, the Fc domain is an IgG₁ Fc domain, particularly a human IgG₁ Fc domain. In one aspect, the Fc domain comprises an amino acid substitution at position P329. In a more specific aspect, the amino acid substitution is P329A or P329G, particularly P329G (numberings according to Kabat EU index). In one aspect, the Fc domain comprises an amino acid substitution at position P329 and a further amino acid substitution at a position selected from E233, L234, L235, N297 and P331 (numberings according to Kabat EU index). In a more specific aspect, the further amino acid substitution is E233P, L234A, L235A, L235E, N297A, N297D or P331S. In particular aspects, the Fc domain comprises amino acid substitutions at positions P329, L234 and L235 (numberings according to Kabat EU index). In more particular aspects, the Fc domain comprises the amino acid mutations L234A, L235A and P329G (“P329G LALA”, “PGLALA” or “LALAPG”). Specifically, in particular aspects, each subunit of the Fc domain comprises the amino acid substitutions L234A, L235A and P329G (Kabat EU index numbering), i.e. in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In some such aspects, the Fc domain is an IgG₁ Fc domain, particularly a human IgG₁ Fc domain. The “P329G LALA” combination of amino acid substitutions almost completely abolishes Fcγ receptor (as well as complement) binding of a human IgG₁ Fc domain, as described in PCT publication no. WO 2012/130831, which is incorporated herein by reference in its entirety. WO 2012/130831 also describes methods of preparing such mutant Fc domains and methods for determining its properties such as Fc receptor binding or effector functions.

IgG₄ antibodies exhibit reduced binding affinity to Fc receptors and reduced effector functions as compared to IgG₁ antibodies. Hence, in one aspect, the Fc domain of the binding molecule(s) of the invention is an IgG₄ Fc domain, particularly a human IgG₄ Fc domain. In one aspect, the IgG₄ Fc domain comprises an amino acid substitution at position S228, specifically the amino acid substitution S228P (numberings according to Kabat EU index). To further reduce its binding affinity to an Fc receptor and/or its effector function, in one aspect, the IgG₄ Fc domain comprises an amino acid substitution at position L235, specifically the amino acid substitution L235E (numberings according to Kabat EU index). In one aspect, the IgG₄ Fc domain comprises an amino acid substitution at position P329, specifically the amino acid substitution P329G (numberings according to Kabat EU index). In a preferred aspect, the IgG₄ Fc domain comprises amino acid substitutions at positions S228, L235 and P329, specifically amino acid substitutions S228P, L235E and P329G (numberings according to Kabat EU index). Such IgG₄ Fc domain mutants and their Fcγ receptor binding properties are described in PCT publication no. WO 2012/130831, incorporated herein by reference in its entirety.

In particular aspects, the Fc domain exhibiting reduced binding affinity to an Fc receptor and/or reduced effector function, as compared to a native IgG₁ Fc domain, is a human IgG₁ Fc domain comprising the amino acid substitutions L234A, L235A and optionally P329G, or a human IgG₄ Fc domain comprising the amino acid substitutions S228P, L235E and optionally P329G (numberings according to Kabat EU index).

In certain aspects, N-glycosylation of the Fc domain has been eliminated. In some such aspects, the Fc domain comprises an amino acid mutation at position N297, particularly an amino acid substitution replacing asparagine by alanine (N297A) or aspartic acid (N297D) (numberings according to Kabat EU index).

In addition to the Fc domains described hereinabove and in PCT publication no. WO 2012/130831, Fc domains with reduced Fc receptor binding and/or effector function also include those with substitution of one or more of Fc domain residues 238, 265, 269, 270, 297, 327 and 329 (U.S. Pat. No. 6,737,056) (numberings according to Kabat EU index). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Mutant Fc domains can be prepared by amino acid deletion, substitution, insertion or modification using genetic or chemical methods well known in the art. Genetic methods may include site specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression.

Alternatively, binding affinity of Fc domains or binding molecule(s) comprising an Fc domain for Fc receptors may be evaluated using cell lines known to express particular Fc receptors, such as human NK cells expressing FcγIIIa receptor.

Effector function of an Fc domain, or a binding molecule comprising an Fc domain, can be measured by methods known in the art. Examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985): U.S. Pat. No. 5,821,337; Bruggemann et al., J Exp Med 166, 1351-1361 (1987).

Alternatively, non-radioactive assays may be employed (see, for example, ACTI™ nonradioactive cytotoxicity assay for flow cytometry (CellTechnology, Inc. Mountain View, CA); and CytoTox 96® non-radioactive cytotoxicity assay (Promega, Madison, WI)). Useful effector cells for such assays include peripheral blood mononuclear cells (PBMC) and Natural Killer (NK) cells.

Alternatively, or additionally, ADCC activity of the molecule of interest may be assessed in vivo, e.g. in an animal model such as that disclosed in Clynes et al., Proc Natl Acad Sci USA 95, 652-656 (1998).

In one aspect, binding of the Fc domain to a complement component, specifically to C1q, is reduced. Accordingly, in one aspect wherein the Fc domain is engineered to have reduced effector function, said reduced effector function includes reduced CDC. C1q binding assays may be carried out to determine whether the Fc domain, or the binding molecule comprising the Fc domain, is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996): Cragg et al., Blood 101, 1045-1052 (2003); and Cragg and Glennie, Blood 103, 2738-2743 (2004)).

FcRn binding and in vivo clearance/half life determinations can also be performed using methods known in the art (see, e.g., Petkova, S. B. et al., Int'l. Immunol. 18 (12): 1759-1769 (2006): WO 2013/120929).

### Configuration of the All-In-One Format

The antigen binding molecule according to the present invention may have various molecular configurations, i.e. the domains of the binding molecule may be linked to each other in different ways.

The all-in-one antigen binding molecule comprises a first and a second target-binding domain, a first and a second cytokine receptor-binding domain and a first and second Fc domain subunit. In a particular aspect, one target-binding domain and one cytokine-receptor domain are fused to one of the Fc domain subunits and the other target-binding domain and cytokine-receptor binding domain are fused the other Fc domain subunit. The cytokine receptor-binding domains may be fused at their C-terminus to the N-terminus of the target-binding domains. The target-binding domains may be fused at their N-terminus or their C-terminus to the C- or N-terminus, respectively, of one of the Fc domain subunits.

In one aspect the target-binding domains are Fab molecules. Thus, the first target-binding domain may comprises a heavy chain variable domain (VH₁), a light chain variable domain (VL₁), a heavy chain constant domain (CH1₁) and a light chain constant domain (CL₁) and the second target-binding domain may comprise a heavy chain variable domain (VH₂), a light chain variable domain (VL₂), a heavy chain constant domain (CH1₂) and a light chain constant domain (CL₂). In one aspect, the first and/or the second target-binding domain is a cross-Fab molecule. In one aspect, the first target-binding domain is a cross-Fab molecule. In one aspect, the second target-binding domain is a cross-Fab molecule. In one aspect, the antigen binding molecule comprises a first and a second target-binding domain, wherein the first target-binding domain is a cross-Fab and the second target-binding domain is a conventional Fab molecule. In one aspect, the antigen binding molecule comprises a first and a second target-binding domain, wherein the first target-binding domain is a conventional Fab molecule and the second target-binding domain is a cross-Fab.

In one aspect, the antigen binding molecule is comprises a first cytokine-receptor binding domain fused at its C-terminus to the N-terminus of VH₁ or VL₁ of the first target-binding domain, and the first target-binding domain is fused at its N-terminus of VH₁ or VL₁ to the C-terminus of the first Fc domain subunit, and the second cytokine receptor-binding domain is fused at its C-terminus to the N-terminus of VH₂ or VL₂ of the second target-binding domain, and the second target-binding domain is fused at its C-terminus of CH1₂ to the N-terminus of the second Fc domain subunit.

In one aspect, the antigen binding molecule comprises a first polypeptide comprising in order from the N-terminus to C-terminus a first Fc domain subunit, a VH₁ and a CH1₁, a second polypeptide comprising in order from the N-terminus to C-terminus a first cytokine receptor-binding domain, a VL₁ and a CL₁, a third polypeptide comprising in order from the N-terminus to C-terminus a VL₂, a CH1₂ and a second Fc domain subunit, and a fourth polypeptide comprising in order from the N-terminus to C-terminus a second cytokine receptor-binding domain, a VH₂ and a CL₂.

In one aspect, the antigen binding molecule comprises a first polypeptide comprising a first polypeptide comprising in order from the N-terminus to C-terminus a first Fc domain subunit, a VL₁ and a CH1₁, a second polypeptide comprising in order from the N-terminus to C-terminus a first cytokine receptor-binding domain, a VH₁ and a CL₁, a third polypeptide comprising in order from the N-terminus to C-terminus a VH₂, a CH1₂ and a second Fc domain subunit, and a fourth polypeptide comprising in order from the N-terminus to C-terminus a second cytokine receptor-binding domain, a VL₂ and a CL₂.

In one aspect, the antigen binding molecule comprises a first polypeptide comprising in order from the N-terminus to C-terminus a first Fc domain subunit, a VL₁ and a CL₁, a second polypeptide comprising in order from the N-terminus to C-terminus a first cytokine receptor-binding domain, a VH₁ and a CH1₁, a third polypeptide comprising in order from the N-terminus to C-terminus a VL₂, a CH1₂ and a second Fc domain subunit, and a fourth polypeptide comprising in order from the N-terminus to C-terminus a second cytokine receptor-binding domain, a VH₂ and a CL₂.

In one aspect, the antigen binding molecule comprises a first polypeptide comprising in order from the N-terminus to C-terminus a first Fc domain subunit, a VH₁ and a CL₁, a second polypeptide comprising in order from the N-terminus to C-terminus a first cytokine receptor-binding domain, a VL₁ and a CH1₁, a third polypeptide comprising in order from the N-terminus to C-terminus a VH₂, a CH1₂ and a second Fc domain subunit, and a fourth polypeptide comprising in order from the N-terminus to C-terminus a second cytokine receptor-binding domain, a VL₂ and a CL₂.

In one aspect, the antigen binding molecule comprises a first polypeptide comprising in order from the N-terminus to C-terminus a first Fc domain subunit, a VH₁ and a CH1₁, a second polypeptide comprising in order from the N-terminus to C-terminus a first cytokine receptor-binding domain, a VL₁ and a CL₁, a third polypeptide comprising in order from the N-terminus to C-terminus a second cytokine receptor-binding domain, a VL₂, a CH1₂ and a second Fc domain subunit, and a fourth polypeptide comprising in order from the N-terminus to C-terminus a VH₂ and a CL₂.

In one aspect, the antigen binding molecule comprises a first polypeptide comprising in order from the N-terminus to C-terminus a first Fc domain subunit, a VL₁ and a CH1₁, a second polypeptide comprising in order from the N-terminus to C-terminus a first cytokine receptor-binding domain, a VH₁ and a CL₁, a third polypeptide comprising in order from the N-terminus to C-terminus a second cytokine receptor-binding domain, a VH₂, a CH1₂ and a second Fc domain subunit, and a fourth polypeptide comprising in order from the N-terminus to C-terminus a VL₂ and a CL₂.

In one aspect, the antigen binding molecule comprises a first polypeptide comprising in order from the N-terminus to C-terminus a first Fc domain subunit, a VL₁ and a CL₁, a second polypeptide comprising in order from the N-terminus to C-terminus a first cytokine receptor-binding domain, a VH₁ and a CH1₁, a third polypeptide comprising in order from the N-terminus to C-terminus a second cytokine receptor-binding domain, a VL₂, a CH1₂ and a second Fc domain subunit, and a fourth polypeptide comprising in order from the N-terminus to C-terminus a VH₂ and a CL₂.

In one aspect, the antigen binding molecule comprises a first polypeptide comprising in order from the N-terminus to C-terminus a first Fc domain subunit, a VH₁ and a CL₁, a second polypeptide comprising in order from the N-terminus to C-terminus a first cytokine receptor-binding domain, a VL₁ and a CH1₁, a third polypeptide comprising in order from the N-terminus to C-terminus a second cytokine receptor-binding domain, a VH₂, a CH1₂ and a second Fc domain subunit.

In one aspect, the antigen binding molecule comprises a first and a second target-binding binding domain that specifically bind to FAP and the first and second cytokine receptor subunits are subunits of the IFNγ receptor complex. In one aspect, the antigen binding molecule comprises a first and a second target-binding binding domain that specifically bind to FAP and the first cytokine receptor-binding domain is capable of binding IFNγR1 and the second cytokine receptor-binding domain is capable of binding IFNγR2. In one aspect, the antigen binding molecule comprises a first and a second target-binding binding domain that specifically bind to FAP and the first cytokine receptor-binding domain is capable of binding IFNγR2 and the second cytokine receptor-binding domain is capable of binding IFNγR1.

In one aspect, the antigen binding molecule comprises a first polypeptide comprising in order from the N-terminus to C-terminus a first Fc domain subunit, a VH₁ and a CH1₁, a second polypeptide comprising in order from the N-terminus to C-terminus a first cytokine receptor-binding domain, a VL₁ and a CL₁, a third polypeptide comprising in order from the N-terminus to C-terminus a VL₂, a CH1₂ and a second Fc domain subunit, and a fourth polypeptide comprising in order from the N-terminus to C-terminus a second cytokine receptor-binding domain, a VH₂ and a CL₂, wherein VH₁, VL₁, CH1₁, and CL₁ form the first target-binding domain and VH₂, VL₂, CH1₂, and CL₂ form the second target-binding domain, wherein the first and second target-binding domains bind FAP, and the first and second cytokine receptor-binding domains bind subunits of the IFNγ receptor complex.

In one aspect, the antigen binding molecule comprises a first polypeptide comprising a first polypeptide comprising in order from the N-terminus to C-terminus a first Fc domain subunit, a VL₁ and a CH1₁, a second polypeptide comprising in order from the N-terminus to C-terminus a first cytokine receptor-binding domain, a VH₁ and a CL₁, a third polypeptide comprising in order from the N-terminus to C-terminus a VH₂, a CH1₂ and a second Fc domain subunit, and a fourth polypeptide comprising in order from the N-terminus to C-terminus a second cytokine receptor-binding domain, a VL₂ and a CL₂, wherein VH₁, VL₁, CH1₁, and CL₁, form the first target-binding domain and VH₂, VL₂, CH1₂, and CL₂, form the second target-binding domain, wherein the first and second target-binding domains bind FAP, and the first and second cytokine receptor-binding domains bind subunits of the IFNγ receptor complex.

In one aspect, the antigen binding molecule comprises a first polypeptide comprising in order from the N-terminus to C-terminus a first Fc domain subunit, a VL₁ and a CL₁, a second polypeptide comprising in order from the N-terminus to C-terminus a first cytokine receptor-binding domain, a VH₁ and a CH1₁, a third polypeptide comprising in order from the N-terminus to C-terminus a VL₂, a CH1₂ and a second Fc domain subunit, and a fourth polypeptide comprising in order from the N-terminus to C-terminus a second cytokine receptor-binding domain, a VH₂ and a CL₂, wherein VH₁, VL₁, CH1₁, and CL₁, form the first target-binding domain and VH₂, VL₂, CH1₂, and CL₂, form the second target-binding domain, wherein the first and second target-binding domains bind FAP, and the first and second cytokine receptor-binding domains bind subunits of the IFNγ receptor complex.

In one aspect, the antigen binding molecule comprises a first polypeptide comprising in order from the N-terminus to C-terminus a first Fc domain subunit, a VH₁ and a CL₁, a second polypeptide comprising in order from the N-terminus to C-terminus a first cytokine receptor-binding domain, a VL₁ and a CH1₁, a third polypeptide comprising in order from the N-terminus to C-terminus a VH₂, a CH1₂ and a second Fc domain subunit, and a fourth polypeptide comprising in order from the N-terminus to C-terminus a second cytokine receptor-binding domain, a VL₂ and a CL₂, wherein VH₁, VL₁, CH1₁, and CL₁ form the first target-binding domain and VH₂, VL₂, CH1₂, and CL₂ form the second target-binding domain, wherein the first and second target-binding domains bind FAP, and the first and second cytokine receptor-binding domains bind subunits of the IFNγ receptor complex.

In one aspect, the antigen binding molecule comprises a first polypeptide comprising in order from the N-terminus to C-terminus a first Fc domain subunit, a VH₁ and a CH1₁, a second polypeptide comprising in order from the N-terminus to C-terminus a first cytokine receptor-binding domain, a VL₁ and a CL₁, a third polypeptide comprising in order from the N-terminus to C-terminus a second cytokine receptor-binding domain, a VL₂, a CH1₂ and a second Fc domain subunit, and a fourth polypeptide comprising in order from the N-terminus to C-terminus a VH₂ and a CL₂, wherein VH₁, VL₁, CH1₁, and CL₁ form the first target-binding domain and VH₂, VL₂, CH1₂, and CL₂ form the second target-binding domain, wherein the first and second target-binding domains bind FAP, and the first and second cytokine receptor-binding domains bind subunits of the IFNγ receptor complex.

In one aspect, the antigen binding molecule comprises a first polypeptide comprising in order from the N-terminus to C-terminus a first Fc domain subunit, a VL₁ and a CH1₁, a second polypeptide comprising in order from the N-terminus to C-terminus a first cytokine receptor-binding domain, a VH₁ and a CL₁, a third polypeptide comprising in order from the N-terminus to C-terminus a second cytokine receptor-binding domain, a VH₂, a CH1₂ and a second Fc domain subunit, and a fourth polypeptide comprising in order from the N-terminus to C-terminus a VL₂ and a CL₂, wherein VH₁, VL₁, CH1₁, and CL₁ form the first target-binding domain and VH₂, VL₂, CH1₂, and CL₂ form the second target-binding domain, wherein the first and second target-binding domains bind FAP, and the first and second cytokine receptor-binding domains bind subunits of the IFNγ receptor complex.

In one aspect, the antigen binding molecule comprises a first polypeptide comprising in order from the N-terminus to C-terminus a first Fc domain subunit, a VL₁ and a CL₁, a second polypeptide comprising in order from the N-terminus to C-terminus a first cytokine receptor-binding domain, a VH₁ and a CH1₁, a third polypeptide comprising in order from the N-terminus to C-terminus a second cytokine receptor-binding domain, a VL₂, a CH1₂ and a second Fc domain subunit, and a fourth polypeptide comprising in order from the N-terminus to C-terminus a VH₂ and a CL₂, wherein VH₁, VL₁, CH1₁, and CL₁ form the first target-binding domain and VH₂, VL₂, CH1₂, and CL₂ form the second target-binding domain, wherein the first and second target-binding domains bind FAP, and the first and second cytokine receptor-binding domains bind subunits of the IFNγ receptor complex.

In one aspect, the antigen binding molecule comprises a first polypeptide comprising in order from the N-terminus to C-terminus a first Fc domain subunit, a VH₁ and a CL₁, a second polypeptide comprising in order from the N-terminus to C-terminus a first cytokine receptor-binding domain, a VL₁ and a CH1₁, a third polypeptide comprising in order from the N-terminus to C-terminus a second cytokine receptor-binding domain, a VH₂, a CH1₂ and a second Fc domain subunit, and a fourth polypeptide comprising in order from the N-terminus to C-terminus a VL₂ and a CL₂, wherein VH₁, VL₁, CH1₁, and CL₁ form the first target-binding domain and VH₂, VL₂, CH1₂, and CL₂ form the second target-binding domain, wherein the first and second target-binding domains bind FAP, and the first and second cytokine receptor-binding domains bind subunits of the IFNγ receptor complex.

In one aspect, the antigen binding molecule comprises a first and a second target-binding binding domain that specifically bind to PD-1 and the first and second cytokine receptor subunits are subunits of the IL-2 receptor complex. In one aspect, the antigen binding molecule comprises a first and a second target-binding binding domain that specifically bind to FAP and the first cytokine receptor-binding domain is capable of binding IL-2Rβ and the second cytokine receptor-binding domain is capable of binding IL-2Rγ. In one aspect, the antigen binding molecule comprises a first and a second target-binding binding domain that specifically bind to FAP and the first cytokine receptor-binding domain is capable of binding IL-2Rγ and the second cytokine receptor-binding domain is capable of binding IL-2Rβ.

In one aspect, the antigen binding molecule comprises a first polypeptide comprising in order from the N-terminus to C-terminus a first Fc domain subunit, a VL₁ and a CL₁, a second polypeptide comprising in order from the N-terminus to C-terminus a first cytokine receptor-binding domain, a VH₁ and a CH1₁, a third polypeptide comprising in order from the N-terminus to C-terminus a VL₂, a CH1₂ and a second Fc domain subunit, and a fourth polypeptide comprising in order from the N-terminus to C-terminus a second cytokine receptor-binding domain, a VH₂ and a CL₂, wherein VH₁, VL₁, CH1₁, and CL₁, form the first target-binding domain and VH₂, VL₂, CH1₂, and CL₂, form the second target-binding domain, wherein the first and second target-binding domains bind PD-1, and the first and second cytokine receptor-binding domains bind subunits of the IL-2 receptor complex.

In one aspect, the antigen binding molecule comprises a first polypeptide comprising in order from the N-terminus to C-terminus a first Fc domain subunit, a VH₁ and a CL₁, a second polypeptide comprising in order from the N-terminus to C-terminus a first cytokine receptor-binding domain, a VL₁ and a CH1₁, a third polypeptide comprising in order from the N-terminus to C-terminus a VH₂, a CH1₂ and a second Fc domain subunit, and a fourth polypeptide comprising in order from the N-terminus to C-terminus a second cytokine receptor-binding domain, a VL₂ and a CL₂, wherein VH₁, VL₁, CH1₁, and CL₁ form the first target-binding domain and VH₂, VL₂, CH1₂, and CL₂ form the second target-binding domain, wherein the first and second target-binding domains bind PD-1, and the first and second cytokine receptor-binding domains bind subunits of the IL-2 receptor complex.

In one aspect, the antigen binding molecule comprises a first polypeptide comprising in order from the N-terminus to C-terminus a first Fc domain subunit, a VH₁ and a CH1₁, a second polypeptide comprising in order from the N-terminus to C-terminus a first cytokine receptor-binding domain, a VL₁ and a CL₁, a third polypeptide comprising in order from the N-terminus to C-terminus a second cytokine receptor-binding domain, a VL₂, a CH1₂ and a second Fc domain subunit, and a fourth polypeptide comprising in order from the N-terminus to C-terminus a VH₂ and a CL₂, wherein VH₁, VL₁, CH1₁, and CL₁ form the first target-binding domain and VH₂, VL₂, CH1₂, and CL₂ form the second target-binding domain, wherein the first and second target-binding domains bind PD-1, and the first and second cytokine receptor-binding domains bind subunits of the IL-2 receptor complex.

In one aspect, the antigen binding molecule comprises a first polypeptide comprising in order from the N-terminus to C-terminus a first Fc domain subunit, a VL₁ and a CH1₁, a second polypeptide comprising in order from the N-terminus to C-terminus a first cytokine receptor-binding domain, a VH₁ and a CL₁, a third polypeptide comprising in order from the N-terminus to C-terminus a second cytokine receptor-binding domain, a VH₂, a CH1₂ and a second Fc domain subunit, and a fourth polypeptide comprising in order from the N-terminus to C-terminus a VL₂ and a CL₂, wherein VH₁, VL₁, CH1₁, and CL₁ form the first target-binding domain and VH₂, VL₂, CH1₂, and CL₂ form the second target-binding domain, wherein the first and second target-binding domains bind PD-1, and the first and second cytokine receptor-binding domains bind subunits of the IL-2 receptor complex.

In one aspect, the antigen binding molecule comprises a first polypeptide comprising in order from the N-terminus to C-terminus a first Fc domain subunit, a VL₁ and a CL₁, a second polypeptide comprising in order from the N-terminus to C-terminus a first cytokine receptor-binding domain, a VH₁ and a CH1₁, a third polypeptide comprising in order from the N-terminus to C-terminus a second cytokine receptor-binding domain, a VL₂, a CH1₂ and a second Fc domain subunit, and a fourth polypeptide comprising in order from the N-terminus to C-terminus a VH₂ and a CL₂, wherein VH₁, VL₁, CH1₁, and CL₁ form the first target-binding domain and VH₂, VL₂, CH1₂, and CL₂ form the second target-binding domain, wherein the first and second target-binding domains bind PD-1, and the first and second cytokine receptor-binding domains bind subunits of the IL-2 receptor complex.

In one aspect, the antigen binding molecule comprises a first polypeptide comprising in order from the N-terminus to C-terminus a first Fc domain subunit, a VH₁ and a CL₁, a second polypeptide comprising in order from the N-terminus to C-terminus a first cytokine receptor-binding domain, a VL₁ and a CH1₁, a third polypeptide comprising in order from the N-terminus to C-terminus a second cytokine receptor-binding domain, a VH₂, a CH1₂ and a second Fc domain subunit, and a fourth polypeptide comprising in order from the N-terminus to C-terminus a VL₂ and a CL₂, wherein VH₁, VL₁, CH1₁, and CL₁ form the first target-binding domain and VH₂, VL₂, CH1₂, and CL₂ form the second target-binding domain, wherein the first and second target-binding domains bind PD-1, and the first and second cytokine receptor-binding domains bind subunits of the IL-2 receptor complex.

The domains of the binding molecule (target antigen-binding domain, cytokine receptor-binding domain and Fc domain) may be fused to each other through one or more peptide linker, especially a (G4S)n peptide linker. A cytokine receptor-binding domain may be linked to a target antigen-binding domain via a (G4S)₁ peptide linker (GGGGS; SEQ ID NO: 118), a (G4S)₂ peptide linker (GGGGSGGGGS; SEQ ID NO: 119), a (G4S)₃ peptide linker (GGGGSGGGGSGGGGS; SEQ ID NO: 120) or a (G4S)₅ peptide linker (GGGGSGGGGSGGGGSGGGGSGGGGS; SEQ ID NO: 121). A target-binding domain may be fused at its N-terminus of its VH or VL to the C-terminus of one of the Fc domain subunits via a (G4S)₁ peptide linker, a (G4S)₂ peptide linker, a (G4S)₃ peptide linker or a (G4S)₅ peptide linker.

In one aspect, the antigen binding molecule comprises a first polypeptide comprising an amino acid sequence of SEQ ID NO: 50, a second polypeptide comprising an amino acid sequence of SEQ ID NO: 48, a third polypeptide comprising an amino acid sequence of SEQ ID NO: 51, and a fourth polypeptide comprising an amino acid sequence of SEQ ID NO: 49. In one aspect, the antigen binding molecule comprises a first polypeptide comprising an amino acid sequence of SEQ ID NO: 99, a second polypeptide comprising an amino acid sequence of SEQ ID NO: 97, a third polypeptide comprising an amino acid sequence of SEQ ID NO: 100, and a fourth polypeptide comprising an amino acid sequence of SEQ ID NO: 98. In one aspect, the antigen binding molecule comprises a first polypeptide comprising an amino acid sequence of SEQ ID NO: 103, a second polypeptide comprising an amino acid sequence of SEQ ID NO: 101, a third polypeptide comprising an amino acid sequence of SEQ ID NO: 104, and a fourth polypeptide comprising an amino acid sequence of SEQ ID NO: 102. In one aspect, the antigen binding molecule comprises a first polypeptide comprising an amino acid sequence of SEQ ID NO: 107, a second polypeptide comprising an amino acid sequence of SEQ ID NO: 105, a third polypeptide comprising an amino acid sequence of SEQ ID NO: 108, and a fourth polypeptide comprising an amino acid sequence of SEQ ID NO: 106. In one aspect, the antigen binding molecule comprises a first polypeptide comprising an amino acid sequence of SEQ ID NO: 99, a second polypeptide comprising an amino acid sequence of SEQ ID NO: 109, a third polypeptide comprising an amino acid sequence of SEQ ID NO: 100, and a fourth polypeptide comprising an amino acid sequence of SEQ ID NO: 110. In one aspect, the antigen binding molecule comprises a first polypeptide comprising an amino acid sequence of SEQ ID NO: 107, a second polypeptide comprising an amino acid sequence of SEQ ID NO: 111, a third polypeptide comprising an amino acid sequence of SEQ ID NO: 113, and a fourth polypeptide comprising an amino acid sequence of SEQ ID NO: 112. In one aspect, the antigen binding molecule comprises a first polypeptide comprising an amino acid sequence of SEQ ID NO: 99, a second polypeptide comprising an amino acid sequence of SEQ ID NO: 114, a third polypeptide comprising an amino acid sequence of SEQ ID NO: 100, and a fourth polypeptide comprising an amino acid sequence of SEQ ID NO: 115. In one aspect, the antigen binding molecule comprises a first polypeptide comprising an amino acid sequence of SEQ ID NO: 107, a second polypeptide comprising an amino acid sequence of SEQ ID NO: 116, a third polypeptide comprising an amino acid sequence of SEQ ID NO: 108, and a fourth polypeptide comprising an amino acid sequence of SEQ ID NO: 117.

In one aspect, the antigen binding molecule comprises a first polypeptide comprising an amino acid sequence of SEQ ID NO: 123, a second polypeptide comprising an amino acid sequence of SEQ ID NO: 122, a third polypeptide comprising an amino acid sequence of SEQ ID NO: 124, and a fourth polypeptide comprising an amino acid sequence of SEQ ID NO: 125.

### Specific Embodiments of the Invention

In the following specific embodiments of the invention are listed.

1. An antigen binding molecule comprising
i) a first target-binding domain,
ii) a second target-binding domain,
iii) a first cytokine receptor-binding domain,
iv) a second cytokine receptor-binding domain, and
v) a Fc domain,
wherein the first target-binding domain is capable of binding a first epitope on the target antigen and the second target-binding domain is capable of binding a second epitope on the target antigen, wherein the first and the second target-binding domains do not compete for binding on the tumor-associated antigen; and
wherein the first cytokine receptor-binding domain is capable of binding a first cytokine receptor subunit and the second cytokine receptor-binding domain is capable of binding a second cytokine receptor subunit.

2. The antigen binding molecule according to embodiment 1, wherein first and second target-binding domains are antibody fragments, particularly Fv, Fab, scFv, scFab or single domain antibodies.

3. The antigen binding molecule according to any one of the preceding embodiments, wherein the first and second target-binding domain are Fab molecules.

4. The antigen binding molecule according to any one of the preceding embodiments, wherein the first target-binding domain comprises a heavy chain variable domain (VH₁), a light chain variable domain (VL₁), a heavy chain constant domain (CH1₁) and a light chain constant domain (CL₁) and the second target-binding domain comprises a heavy chain variable domain (VH₂), a light chain variable domain (VL₂), a heavy chain constant domain (CH1₂) and a light chain constant domain (CL₂).

5. The antigen binding molecule according to any one of the preceding embodiments, wherein the first target-binding domain and/or the second target-binding domain is a cross-Fab molecule.

6. The antigen binding molecule according to any one of the preceding embodiments, wherein the first target-binding domain and/or the second target-binding domain comprise charge mutations.

7. The antigen binding molecule according to any one of the preceding embodiments, wherein the first target-binding domain is a cross-Fab and the second target-binding domain comprises charge mutations or wherein the second target-binding domain is a cross-Fab and the first target-binding domain comprises charge mutations.

8. The antigen binding molecule according to any one of the preceding embodiments, wherein the first target-binding domain and the second target-binding domain specifically bind to a tumor-associated antigen or a T cell antigen.

9. The antigen binding molecule according to any one of the preceding embodiments, wherein the first target-binding domain and the second target-binding domain specifically bind to FAP, PD-1, Her2, Her3, LAG-3, CEA or EGFR.

10. The antigen binding molecule according to any one of the preceding embodiments, wherein the first target-binding domain and the second target-binding domain specifically bind to FAP or PD-1.

11. The antigen binding molecule according to any one of the preceding embodiments, wherein
a) the first target-binding domain comprises a VH₁ of SEQ ID NO: 20 and a VL₁ of SEQ ID NO: 21 and the second target-binding domain comprises a VH₂ of SEQ ID NO: 22 and a VL₂ of SEQ ID NO: 23, or
b) the first target-binding domain comprises a VH₁ of SEQ ID NO: 22 and a VL₁ of SEQ ID NO: 23 and the second target-binding domain comprises a VH₂ of SEQ ID NO: 20 and a VL₂ of SEQ ID NO: 21, or
c) the first target-binding domain comprises a VH₁ of SEQ ID NO: 80 and a VL₁ of SEQ ID NO: 81 and the second target-binding domain comprises a VH₂ of SEQ ID NO: 82 and a VL₂ of SEQ ID NO: 83, or
d) the first target-binding domain comprises a VH₁ of SEQ ID NO: 82 and a VL₁ of SEQ ID NO: 83 and the second target-binding domain comprises a VH₂ of SEQ ID NO: 80 and a VL₂ of SEQ ID NO: 81; or
e) the first target-binding domain comprises a VH₁ of SEQ ID NO: 82 and a VL₁ of SEQ ID NO: 83 and the second target-binding domain comprises a VH₂ of SEQ ID NO: 140 and a VL₂ of SEQ ID NO: 141; or
f) the first target-binding domain comprises a VH₁ of SEQ ID NO: 140 and a VL₁ of SEQ ID NO: 141 and the second target-binding domain comprises a VH₂ of SEQ ID NO: 82 and a VL₂ of SEQ ID NO: 83.

12. The antigen binding molecule according to any one of the preceding embodiments, wherein both the first and second cytokine receptor subunits are subunits of the IFNγ receptor complex or IL-2 receptor complex.

13. The antigen binding molecule according to any one of the preceding embodiments, wherein
a) the first cytokine receptor-binding domain is capable of binding IFNγR1 and the second cytokine receptor-binding domain is capable of binding IFNγR2, or
b) the first cytokine receptor-binding domain is capable of binding IFNγR2 and the second cytokine receptor-binding domain is capable of binding IFNγR1;
c) the first cytokine receptor-binding domain is capable of binding IL-2Rβ and the second cytokine receptor-binding domain is capable of binding IL-2Rγ; or
d) the first cytokine receptor-binding domain is capable of binding IL-2Rγ and the second cytokine receptor-binding domain is capable of binding IL-2Rβ.

14. The antigen binding molecule according to any one of the preceding embodiments, wherein the first and second cytokine receptor-binding domains are antibody fragments, particularly Fv, Fab, scFv, scFab, single domain antibodies or VHH domains.

15. The antigen binding molecule according to any one of the preceding embodiments, wherein the first and second cytokine receptor-binding domains are VHH domains.

16. The antigen binding molecule according to any one of the preceding embodiments, wherein
a) the first cytokine receptor-binding domain comprises an amino acid sequence selected from SEQ ID NO: 1, SEQ ID NO: 2, SEQ ID NO: 3 and SEQ ID NO: 5, and the second cytokine receptor-binding domain comprises an amino acid sequence selected from SEQ ID NO: 6, SEQ ID NO: 7 and SEQ ID NO: 9, or
b) the first cytokine receptor-binding domain comprises an amino acid sequence selected from SEQ ID NO: 6, SEQ ID NO: 7 and SEQ ID NO: 9, and the second cytokine receptor-binding domain comprises an amino acid sequence selected from SEQ ID NO: 1, SEQ ID NO: 2, SEQ ID NO: 3 and SEQ ID NO: 5; or
c) the first cytokine receptor-binding domain comprises an amino acid sequence selected from SEQ ID NO: 60, SEQ ID NO: 61, SEQ ID NO: 62 and SEQ ID NO: 64, and the second cytokine receptor-binding domain comprises an amino acid sequence selected from SEQ ID NO: 66, SEQ ID NO: 67, SEQ ID NO: 68 and SEQ ID NO: 69, or
d) the first cytokine receptor-binding domain comprises an amino acid sequence selected from SEQ ID NO: 66, SEQ ID NO: 67, SEQ ID NO: 68 and SEQ ID NO: 69, and the second cytokine receptor-binding domain comprises an amino acid sequence selected from SEQ ID NO: 60, SEQ ID NO: 61, SEQ ID NO: 62 and SEQ ID NO: 64.

17. The antigen binding molecule according to any one of the preceding embodiments, wherein the Fc domain comprises a first Fc domain subunit and a second Fc domain subunit.

18. The antigen binding molecule according to any one of the preceding embodiments, wherein the Fc domain is an IgG, particularly an IgG₁, Fc domain.

19. The antigen binding molecule according to any one of the preceding embodiments, wherein the Fc domain is a human Fc domain.

20. The antigen binding molecule according to any one of the preceding embodiments, wherein the Fc domain comprises a modification promoting the association of the first and the second subunit of the Fc domain.

21. The antigen binding molecule according to any one of the preceding embodiments, wherein the Fc domain comprises one or more amino acid substitution that reduces binding to an Fc receptor and/or effector function.

22. The antigen binding molecule according to any one of the preceding embodiments, wherein
the first cytokine receptor-binding domain is fused at its C-terminus to the N-terminus of VH₁ or VL₁ of the first target-binding domain, and the first target-binding domain is fused at its N-terminus of VH₁ or VL₁ to the C-terminus of the first Fc domain subunit, and the second cytokine receptor-binding domain is fused at its C-terminus to the N-terminus of VH₂ or VL₂ of the second target-binding domain, and the second target-binding domain is fused at its C-terminus of CH1₂ or CL₁ to the N-terminus of the second Fc domain subunit.

23. The antigen binding molecule according to any one of the preceding embodiments, comprising
a) a first polypeptide comprising in order from the N-terminus to C-terminus a first Fc domain subunit, a VH₁ and a CH1₁, a second polypeptide comprising in order from the N-terminus to C-terminus a the first cytokine receptor-binding domain, a VL₁ and a CL₁, a third polypeptide comprising in order from the N-terminus to C-terminus a VL₂, a CH1₂ and a second Fc domain subunit, and a fourth polypeptide comprising in order from the N-terminus to C-terminus a the second cytokine receptor-binding domain, a VH₂ and a CL₂; or
b) a first polypeptide comprising in order from the N-terminus to C-terminus a first Fc domain subunit, a VL₁ and a CH1₁, a second polypeptide comprising in order from the N-terminus to C-terminus a the first cytokine receptor-binding domain, a VH₁ and a CL₁, a third polypeptide comprising in order from the N-terminus to C-terminus a VH₂, a CH1₂ and a second Fc domain subunit, and a fourth polypeptide comprising in order from the N-terminus to C-terminus a the second cytokine receptor-binding domain, a VL₂ and a CL₂; or
c) a first polypeptide comprising in order from the N-terminus to C-terminus a first Fc domain subunit, a VL₁ and a CL₁, a second polypeptide comprising in order from the N-terminus to C-terminus a the first cytokine receptor-binding domain, a VH₁ and a CH1₁, a third polypeptide comprising in order from the N-terminus to C-terminus a VL₂, a CH1₂ and a second Fc domain subunit, and a fourth polypeptide comprising in order from the N-terminus to C-terminus a the second cytokine receptor-binding domain, a VH₂ and a CL₂; or
d) a first polypeptide comprising in order from the N-terminus to C-terminus a first Fc domain subunit, a VH₁ and a CL₁, a second polypeptide comprising in order from the N-terminus to C-terminus a the first cytokine receptor-binding domain, a VL₁ and a CH1₁, a third polypeptide comprising in order from the N-terminus to C-terminus a VH₂, a CH1₂ and a second Fc domain subunit, and a fourth polypeptide comprising in order from the N-terminus to C-terminus a the second cytokine receptor-binding domain, a VL₂ and a CL₂; or
e) a first polypeptide comprising in order from the N-terminus to C-terminus a first Fc domain subunit, a VH₁ and a CH1₁, a second polypeptide comprising in order from the N-terminus to C-terminus a the first cytokine receptor-binding domain, a VL₁ and a CL₁, a third polypeptide comprising in order from the N-terminus to C-terminus a the second cytokine receptor-binding domain, a VL₂, a CH1₂ and a second Fc domain subunit, and a fourth polypeptide comprising in order from the N-terminus to C-terminus a VH₂ and a CL₂; or
f) a first polypeptide comprising in order from the N-terminus to C-terminus a first Fc domain subunit, a VL₁ and a CH1₁, a second polypeptide comprising in order from the N-terminus to C-terminus a the first cytokine receptor-binding domain, a VH₁ and a CL₁, a third polypeptide comprising in order from the N-terminus to C-terminus a the second cytokine receptor-binding domain, a VH₂, a CH1₂ and a second Fc domain subunit, and a fourth polypeptide comprising in order from the N-terminus to C-terminus a VL₂ and a CL₂; or
g) a first polypeptide comprising in order from the N-terminus to C-terminus a first Fc domain subunit, a VL₁ and a CL₁, a second polypeptide comprising in order from the N-terminus to C-terminus a the first cytokine receptor-binding domain, a VH₁ and a CH1₁, a third polypeptide comprising in order from the N-terminus to C-terminus a the second cytokine receptor-binding domain, a VL₂, a CH1₂ and a second Fc domain subunit, and a fourth polypeptide comprising in order from the N-terminus to C-terminus a VH₂ and a CL₂; or
h) a first polypeptide comprising in order from the N-terminus to C-terminus a first Fc domain subunit, a VH₁ and a CL₁, a second polypeptide comprising in order from the N-terminus to C-terminus a the first cytokine receptor-binding domain, a VL₁ and a CH1₁, a third polypeptide comprising in order from the N-terminus to C-terminus a the second cytokine receptor-binding domain, a VH₂, a CH1₂ and a second Fc domain subunit, and a fourth polypeptide comprising in order from the N-terminus to C-terminus a VL₂ and a CL₂; wherein VH₁, VL₁, CH1₁, and CL₁ form the first target-binding domain and VH₂, VL₂, CH1₂, and CL₂ form the second target-binding domain.

24. The antigen binding molecule according to any one of the preceding embodiments, wherein the first target-binding domain and the second target-binding domain specifically bind to FAP and the first and second cytokine receptor subunits are subunits of the IFNγ receptor complex.

25. The antigen binding molecule according to any one of the preceding embodiments, comprising
a) a first polypeptide comprising the amino acid sequence of SEQ ID NO: 50, a second polypeptide comprising the amino acid sequence of SEQ ID NO: 48, a third polypeptide comprising the amino acid sequence of SEQ ID NO: 51 and a fourth polypeptide comprising the amino acid sequence of SEQ ID NO: 49;
b) a first polypeptide comprising an amino acid sequence of SEQ ID NO: 99, a second polypeptide comprising an amino acid sequence of SEQ ID NO: 97, a third polypeptide comprising an amino acid sequence of SEQ ID NO: 100, and a fourth polypeptide comprising an amino acid sequence of SEQ ID NO: 98;
c) a first polypeptide comprising an amino acid sequence of SEQ ID NO: 103, a second polypeptide comprising an amino acid sequence of SEQ ID NO: 101, a third polypeptide comprising an amino acid sequence of SEQ ID NO: 104, and a fourth polypeptide comprising an amino acid sequence of SEQ ID NO: 102;
d) a first polypeptide comprising an amino acid sequence of SEQ ID NO: 107, a second polypeptide comprising an amino acid sequence of SEQ ID NO: 105, a third polypeptide comprising an amino acid sequence of SEQ ID NO: 108, and a fourth polypeptide comprising an amino acid sequence of SEQ ID NO: 106;
e) a first polypeptide comprising an amino acid sequence of SEQ ID NO: 99, a second polypeptide comprising an amino acid sequence of SEQ ID NO: 109, a third polypeptide comprising an amino acid sequence of SEQ ID NO: 100, and a fourth polypeptide comprising an amino acid sequence of SEQ ID NO: 110;
f) a first polypeptide comprising an amino acid sequence of SEQ ID NO: 107, a second polypeptide comprising an amino acid sequence of SEQ ID NO: 111, a third polypeptide comprising an amino acid sequence of SEQ ID NO: 113, and a fourth polypeptide comprising an amino acid sequence of SEQ ID NO: 112;
g) a first polypeptide comprising an amino acid sequence of SEQ ID NO: 99, a second polypeptide comprising an amino acid sequence of SEQ ID NO: 114, a third polypeptide comprising an amino acid sequence of SEQ ID NO: 100, and a fourth polypeptide comprising an amino acid sequence of SEQ ID NO: 115;
h) a first polypeptide comprising an amino acid sequence of SEQ ID NO: 107, a second polypeptide comprising an amino acid sequence of SEQ ID NO: 116, a third polypeptide comprising an amino acid sequence of SEQ ID NO: 108, and a fourth polypeptide comprising an amino acid sequence of SEQ ID NO: 117.

26. The antigen binding molecule according to any one of embodiments 1 to 23, wherein the first target-binding domain and the second target-binding domain specifically bind to PD-1 and the first and second cytokine receptor subunits are subunits of the IL-2 receptor complex.

27. The antigen binding molecule according to embodiments 25, comprising a first polypeptide comprising an amino acid sequence of SEQ ID NO: 123, a second polypeptide comprising an amino acid sequence of SEQ ID NO: 122, a third polypeptide comprising an amino acid sequence of SEQ ID NO: 124, and a fourth polypeptide comprising an amino acid sequence of SEQ ID NO: 125.

### EXAMPLES

### Example 1: Generation of Binders for Cytokine Receptor Subunits

### 1.1 Generation of Antigens for Immunization and Phage Display

For llama immunization, the extracellular domain (ECD) of human IFNγR1 subunit and the ECD of human IFNγR2 subunit were co-expressed as an Fc-tagged heterodimer based on the knobs-into-holes technology (FIG. 1A, P1AG0843). Likewise, the ECD of human IL-2Rβ (CD122) subunit and human IL-2Rγ (common gamma chain: CD132) subunit were co-expressed as an Fc-tagged heterodimer (FIG. 1D, P1AE2657). The C-terminus of IFNγR1 ECD was fused to Fc knob via a G₄SG₄ linker. An AviTag was included at the C-terminus of the Fc for site-specific biotinylation during co-expression with BirA biotin ligase. The IFNγR2 ECD containing C174S substitution was fused to Fc hole with a G₄SG₄ linker and the construct contained a C-terminal Twin-Strep-tag. The C-terminus of IL-2Rβ ECD was fused to Fc knob via a GAQ linker. An AviTag was included at the C-terminus of the Fc for site-specific biotinylation during co-expression with BirA biotin ligase. The IL-2Rγ ECD was C-terminally fused to Fc hole with a GAQ linker.

For phage display, monovalent IFNγR1 ECD fused to biotinylated Fc knob paired with empty Fc hole (FIG. 1B, P1AG1987) and monovalent IFNγR2 ECD C174S fused to Fc hole paired with empty biotinylated Fc knob (FIG. 1C, P1AG6735), as well as monovalent IL-2Rβ ECD fused to biotinylated Fc knob paired with empty Fc hole (FIG. 1E, P1AF1104) and monovalent IL-2Rγ ECD fused to Fc hole paired with empty biotinylated Fc knob (FIG. 1F, P1AF1106), were generated. All antigens were produced in the Expi293 mammalian expression system (Expi293™ Expression System Kit, Thermo Fisher Scientific, Cat. No: A14635) following manufacturer's recommendation. Supernatants were harvested by centrifugation, filtered and purified by Protein A affinity chromatography (Protein A MabSelect™ SuRe™, Cytiva, Cat. No: 17543803) followed by size exclusion chromatography (SEC) (HiLoad S200 16/600, Cytiva, Cat. No: 28989335) according to manufacturer's recommendation.

### 1.2 VHH Library Construction

The VHH library was constructed by ProSci Inc. using ˜3×10⁸ peripheral blood mononuclear cells (PBMCs) from bleed 2 and ˜6.6×10⁸ PBMCs bleed 3 of a single llama. The library was provided in the TG1 E. coli strain encoded in a derivative of the pADL-23c phagemid vector (Antibody Design Labs, San Diego, CA), incorporated by approximately ˜1×10⁹ independent electroporation events with >95% insert efficiency. Libraries were provided as ˜3.2×10¹⁰ cfu/mL E. coli in 2×YT medium containing 20% glycerol.

### 1.3 Generation of VHH Phage Library

E. coli containing the VHH-encoded phagemid library were inoculated in 2×YT medium containing 100 μg/ml ampicillin and 1% glucose. Following infection with VCSM13 helper phage, E. coli were grown in 2×YT medium containing 100 μg/ml ampicillin and 50 μg/ml kanamycin. Following overnight incubation on a shaker at 30° C., cultures were harvested by centrifugation at 8000 rpm for 20 minutes and supernatants were collected. Phage were precipitated in 20% PEG/2.5 M NaCl solution and incubated on ice for 1 hour. Precipitated phage were centrifuged at 8000 rpm for 20 minutes and pellets were resuspended in ddH₂O followed by precipitation in PEG/NaCl solution for 1 hour on ice. Phage were centrifuged at 8000 rpm for 20 minutes and pellets were resuspended in PBS containing 20% glycerol.

### 1.4 Isolation of IFNγR1-Specific, IFNγR2-Specific, IL-2Rβ-Specific and IL-2Rγ-Specific VHHs by Phage Display

To selectively enrich for antigen-specific binders and prevent the enrichment of Fc-specific binders. Fc-competitive soluble selections were performed against human IFNγR1 antigen (P1AG1987), human IFNγR2 antigen (P1AG6735), human IL-2Rβ antigen (P1AF1104) and human IL-2Rγ antigen (P1AF1106), in the presence of human Fc (FIG. 1G. P1AD4290). An aliquot of 1×10¹¹ phage from the llama-derived VHH library was used per antigen. Phage and magnetic streptavidin beads (Dynabeads; M-280) Streptavidin, Invitrogen, Cat. No: 11205D) were independently blocked with 1% BSA, 0.1% Tween-20 in phosphate-buffered saline (PBS) to reduce non-specific interactions. All incubations were performed for one hour at 20° C. unless otherwise stated. The biotinylated, Fc-tagged antigens were added at 100 nM concentration to blocked phage in the presence of excess amounts (500 nM) of non-biotinylated Fc (FIG. 2). Following incubation, pre-blocked Dynabeads were added to capture biotinylated antigens of interest and bound VHH-expressing phage. Fc-specific and non-specific phage were removed by 5-10 wash steps in PBS containing 0.1% Tween-20 followed by PBS using a KingFisher magnetic particle processor (Thermo Fisher Scientific). Antigen-specific phage were eluted in 800 μl of 100 mM trimethylamine (TEA) and neutralized by addition of 400 μl of 1 M Tris pH 7.4. Mid-logarithmic phase TG1 E. coli in 2×YT medium were infected with eluted phage, followed by infection with VCSM13 helper phage. Following addition of 100 μg/ml ampicillin and 50 μg/ml kanamycin, infected E. coli were incubated overnight on a shaker at 30° C. Phage were precipitated with PEG/NaCl and used for the next round. Selections were performed for a total of 3 rounds using decreasing target antigen concentrations (100 nM, 50 nM, 25 nM) while soluble Fc concentrations remained constant (500 nM) (FIG. 2).

### 1.5 Verification of Target Specificity by ELISA

Round 3 enrichment libraries were screened for target specificity vs Fc specificity by ELISA. Individual VHH clones were expressed in 1 ml E. coli cultures in a 96-well plate and supernatants containing soluble VHH were screened for specificity by ELISA. Supernatants were transferred to neutravidin 96-well plates coated with biotinylated Fc (P1AE6073) or target Fc-tagged biotinylated antigen (P1AG1987. P1AG6735, P1AF1104 or P1AF1106). Following incubation for 1 hour at 20° C. plates were washed 3 times with PBST and 3 times with PBS. HRP-conjugated anti-His antibody (Sigma. Cat. No: A7058) was added to wells at 1:2000 dilution and plates were incubated for 1 hour at room temperature. Plates were washed 3 times with PBST followed by 3 washes with PBS. Plates were developed by adding 1-Step Ultra TMB-ELISA substrate (Thermo Fisher Scientific. Cat. No: 34028) and reactions were quenched with sulfuric acid. Absorbance at 450 nm was measured using a Tecan infinite M1000 Pro and reference absorbance values were measured at 650) nm. Reference-subtracted absorbance values at 450 nm were represented as stacked bar charts for IFNγR1-specific VHHs (FIG. 3A). IFNγR2-specific VHHs (FIG. 3B). IL-2Rβ-specific VHHs (FIG. 3C) and IL-2Rγ-specific VHHs (FIG. 3D). ELISA data suggested >90% of clones were target-specific and VHH from round 3 enrichment libraries were subsequently cloned into bispecific heavy chain antibody formats.

### Example 2: Evaluation of Agonist Activity Towards IFNγ and IL-2 Receptors

2.1 Assembly of Enriched VHH Libraries into Heavy Chain Antibody Formats

IFNγR activation is achieved by IFNγ-mediated cross-linking of the IFNγR1 and IFNγR2 receptor chains. To mimic the assembly in an IFNγ-independent manner, a VHH domain with IFNγR1 specificity was paired with an IFNγR2-specific VHH by fusing to knob-into-hole Fc chains via a flexible linker generating IFNγR1-IFNγR2 bispecific heavy chain antibodies. IL-2R activation is achieved by IL2-mediated cross-linking of the IL-2Rβ and IL-2Rγ receptor chains. Thus, to mimic the assembly in an IL2-independent manner, a VHH domain with IL-2Rβ specificity was paired with an IL-2Rγ-specific VHH by fusing to knob-into-hole Fc chains via a flexible linker generating IL-2Rβ-IL-2Rγ bispecific heavy chain antibodies. To this end, the round 3 VHH library enriched for IFNγR1- or IL-2Rβ-specificity were cloned in bulk using Gibson assembly to achieve format conversion of VHH by fusing to Fc knob via a 5 (G₄S) linker (FIG. 4A). In the same way, the round 3 IFNγR2- or IL-2Rγ-specific VHH library were fused to Fc hole via a 5 (G₄S) linker using Gibson assembly (FIG. 4B). During the assembly, a suitable signal sequence and vector compatible with mammalian expression were included. Primers, PCR conditions and Gibson assembly reactions were defined as per New England Biolabs' NEBuilder HiFi DNA Assembly protocol (Cat. No: E5510). Following bacterial transformation, colonies were selected at random and five unique sequences from each specificity pool were selected. Using the Expi293 system, a 5×5 matrix was generated to express unique knob-into-holes bispecific heavy chain antibodies comprising IFNγR1 and IFNγR2 VHH pairs and 25 unique knob-into-holes bispecific heavy chain antibodies comprising IL-2Rβ VHH and IL-2Rγ VHH pairs (FIG. 5). Expi293 cells were transiently transfected following manufacturer's recommendation (Expi293™ Expression System Kit, Thermo Fisher Scientific, Cat. No: A14635) and supernatants containing secreted soluble bispecific heavy chain antibodies were collected. The bispecific heavy chain antibodies were purified by Protein A resin and further characterized for functional activity using reporter cell assays.

### 2.2 Functional Characterization of Bispecific Heavy Chain Antibodies

The HEK Blue IFNγ reporter cell assay (Invivogen) was used to screen bispecific heavy chain antibodies comprising different IFNγR1 and IFNγR2 VHH pairs and identify IFNγR agonists. Whereas, the HEK Blue IL-2 reporter cell assay (Invivogen) was used to screen bispecific heavy chain antibodies comprising different IL-2Rβ and IL-2Rγ VHH pairs and identify IL-2R agonists. The in vitro HEK Blue IFNγ reporter cell system (Invivogen, Cat. No: hkb-ifng) reports IFNγR signaling via a STAT1-inducible secreted alkaline phosphatase (SEAP) system. While the in vitro HEK Blue IL-2 reporter cell (Invivogen, Cat. No: hkb-il2) recapitulates the human IL-2R signaling pathway via expression of the IL-2R chains (IL-2Rα, β and γ subunits) and effectors of the downstream signaling cascade (JAK3 and STAT5) and a STAT5-inducible secreted alkaline phosphatase (SEAP) reporter system. Upon addition of QuantiBlue substrate (Invivogen, Cat. No: rep-qbs), absorbance at 650 nm is measured, correlating with SEAP levels and IFNγR and IL-2R activity, respectively. In the initial screening, supernatants from the Expi293 5×5 expression matrix containing soluble IFNγR1-IFNγR2 or IL-2Rβ-IL-2Rγ bispecific heavy chain antibodies were incubated with HEK Blue IFNγ or HEK Blue IL-2 reporter cells, respectively. Following a 20 hour incubation, IFNγR and IL-2 activity was quantified by addition of QuantiBlue reagent (Invivogen, Cat. No: rep-qbs), according to the manufacturer's recommendation. The absorbance at 650 nm was measured, with higher absorbance levels indicating increased IFNγR or IL-2 signaling.

Of the 25 tested IFNγR1-IFNγR2 bispecific heavy chain antibodies (format depicted in FIG. 5), 14 showed IFNγR activity (FIG. 6A). Thus, 14 pairs of IFNγR1- and IFNγR2-VHH domains demonstrated IFNγ agonistic activity. Of the 25 tested IL-2Rβ-IL-2Rγ bispecific heavy chain antibodies, 9 pairs containing non-specific VHH domains (IL2Rβ_4 and IL2Rγ_1) did not induce IL-2R activity. The other 16 pairs, which were formed of functional IL-2Rβ and IL-2Rγ binders, all showed IL-2R agonism (FIG. 6B). Thus, 16 pairs of IL-2Rβ- and IL-2Rγ-VHH domains demonstrated IL-2 agonistic activity.

Next, purified agonistic IFNγR1-IFNγR2 bispecific heavy chain antibodies (P1AH1877-P1AH1890, see Table 1) were tested for dose-dependent IFNγR signaling and agonistic IL-2Rβ-IL-2Rγ bispecific heavy chain antibodies were tested for dose-dependent IL-2R signaling (see Table 2).

TABLE 1
IFNγR1-IFNγR2 bispecific heavy chain antibodies generated by the 5x5
expression matrix showing IFNγ agonistic activity in FIG. 6A.

| | IFNγR2_1 | IFNγR2_2 | IFNγR2_3 | IFNγR2_4 | IFNγR2_5 |
|---|---|---|---|---|---|
| | SEQ ID NO: 15 | SEQ ID NO: 16 | SEQ ID NO: 17 | SEQ ID NO: 18 | SEQ ID NO: 19 |
| IFNγR1_1 | P1AH1877 | P1AH1878 | | P1AH1879 | |
| SEQ ID NO: 11 | | | | | |
| IFNγR1_2 | P1AH1880 | P1AH1881 | | P1AH1882 | |
| SEQ ID NO: 12 | | | | | |
| IFNγR1_3 | P1AH1883 | P1AH1884 | | P1AH1885 | |
| SEQ ID NO: 13 | | | | | |
| IFNγR1_4 | | | | | |
| SEQ ID NO: 59 | | | | | |
| IFNγR1_5 | P1AH1886 | P1AH1887 | P1AH1889 | P1AH1888 | P1AH1890 |
| SEQ ID NO: 14 | | | | | |

A concentration range spanning from 100 nM to 0.01 nM with 10-fold serial dilution steps was used with HEK Blue IFNγ cells according to the Invivogen HEK Blue protocol. Absorbance at 650 nm was measured and responses were plotted using GraphPad software (FIG. 7). Graphs were separated according to VHH clone with IFNγR1 specificity, with recombinant human IFNγ (recIFNγ; SEQ ID NO: 26) and anti-FAP IgG fused to IFNγ homodimer (P1AF3574; SEQ ID NO: 24 and SEQ ID NO: 25) serving as reference molecules. The variety of responses observed in all graphs, ranging from potent IFNγ mimetics to weak agonists, demonstrated an interplay between both the IFNγR1 and IFNγR2 binders. EC50 values were derived using GraphPad Prism software (FIG. 8), illustrating the breadth of agonistic activities and underlining the potential for VHH pairs to achieve potent yet tunable cytokine mimetics.

TABLE 2
IL-2Rβ-IL-2Rγ bispecific heavy chain antibodies generated by
the 5x5 expression matrix showing IL-2R activity in FIG. 6B.

| | | | | |
|---|---|---|---|---|
| IL2Rγ_1 | IL2Rγ_2 | IL2Rγ_3 | IL2Rγ_4 | IL2Rγ_5 |
| SEQ ID NO: 79 | SEQ ID NO: 74 | SEQ ID NO: 75 | SEQ ID NO: 76 | SEQ ID NO: 77 |

| | | | | |
|---|---|---|---|---|
| IL2Rβ_1 | P1AH1165 | P1AH1166 | P1AH1167 | P1AH1168 |
| SEQ ID NO: 70 | | | | |
| IL2Rβ_2 | P1AH1169 | P1AH1170 | P1AH1171 | P1AH1172 |
| SEQ ID NO: 71 | | | | |
| IL2Rβ_3 | P1AH1173 | P1AH1174 | P1AH1175 | P1AH1176 |
| SEQ ID NO: 72 | | | | |
| IL2Rβ_4 | | | | |
| SEQ ID NO: 78 | | | | |
| IL2Rβ_5 | P1AH1177 | P1AH1178 | P1AH1179 | P1AH1180 |
| SEQ ID NO: 73 | | | | |

A concentration range of 20 nM, 0.8 nM and 0.032 nM was used with HEK Blue IL-2 reporter cells according to the the Invivogen HEK Blue protocol. Agonistic activity of the IL-2 mimetics was compared with IL-2v fused to anti-PD1 IgG (PD1-IL2v: P1AE4422). The VHH pairs displayed potent IL-2R agonism in a dose-dependent manner (FIG. 9). Moreover, in contrast to the natural cytokine in which agonism is mediated by a single molecule, the use of a VHH pair to activate the receptor provided opportunities for conditionally active IL-2 mimetics via molecular split approaches.

To further characterize the agonistic activity of the 14 functional IFNγR1- and IFNγR2-VHH pairs, various tumor cell lines with previously identified responsiveness to IFNγ (MKN45, BxPC-3, COR-L105: data not shown) were selected to explore therapeutically relevant downstream effects of IFNγR signaling which may have the capacity to modulate the tumor microenvironment (TME). In the first instance, MHC-I and PD-L1 were selected as tumor cell surface biomarkers to characterize the TME-modulating potential of functional bispecific VHH pairs. MHC-I upregulation is a downstream response following IFNγR stimulation and may provide an increase in antigenicity on tumor cells, while PD-L1 upregulation is another downstream indicator of functional IFNγ signaling. To assess the capacity of VHH pairs to induce IFNγ signaling in vitro, tumor cells were incubated with the 14 previously tested heavy chain antibodies (P1AH1877-P1AH1890) in concentration series ranging from 100 nM to 0.1 nM. After 72 hours, MHC-I and PD-L1 expression levels were quantified by flow cytometry and data were analyzed using FlowJo software. Mean fluorescent intensity (MFI) values were blank-subtracted and normalized to recombinant IFNγ responses. Upregulation of MHC-I and PD-L1 was measured in three tumor cell types: MKN45 (FIG. 10A, 10B), BxPC-3 (FIGS. 10C, 10D) and COR-L105 (FIG. 10E, 10F). A variety of responses was observed, corroborating the broad range of agonistic activities observed previously in HEK Blue IFNγ reporter cells. One notable observation was an often decreased PD-L1 expression compared to MHC-I expression induced by certain VHH pairs, suggesting a complexity in IFNγ signaling that may engage different pathways and highlighting the potential for agonistic VHH pairs to elicit a bias towards desired phenotypic effects.

The induction of IFNγR signaling via an agonistic IFNγR VHH pair and IL-2R signaling via an agonistic IL-2R VHH pair provided opportunities to engineer conditionally active IFNγ and IL-2 mimetics. As a starting point for a conditional IFNγR agonist, P1AH1884 was selected as a potent IFNγ mimetic (FIG. 8) with activity closely correlating with recombinant IFNγ responses across all tumor lines tested (FIG. 9). For a conditional IL-2R agonist P1AH1177 was selected.

The corresponding VHH pairs were further engineered to generate conditionally active agonists.

### Example 3: Engineering of Conditionally Active IFNγ and IL-2 Mimetics

### 3.1 FAP-Dependent Biparatopic Assembly of Split Dual-Targeted IFNγ Mimetics

To achieve conditional immunomodulation within an immune-deserted tumor microenvironment, cell surface markers reportedly expressed in non-inflamed tumor phenotypes were further explored. Fibroblast activation protein (FAP), a serine protease described with high expression in cancer-associated stromal tissue of immune-deserted tumors, was selected as a target to achieve cold tumor penetration. As a next step, a pair of agonistic VHH domains mimicking IFNγ were distributed onto two distinct molecules to inactivate the VHH pair, whilst incorporating a FAP-dependent assembly strategy to restore agonistic VHH activity in the presence of FAP (FIG. 11A). To this end, two proximal epitopes on the FAP antigen were selected and the corresponding non-competing anti-FAP binders were engineered to mediate assembly of an agonistic VHH pair. In the first instance, VHH domains of the P1AH1884 heavy chain antibody were fused to the N-terminus of the VH or VL domains of the anti-FAP binders via a flexible 5 (G₄S) linker, resulting in 8 possible format configurations (FIG. 11B-I). To test for FAP-dependent IFNγR signaling, upregulation of MHC-I and PD-L1 was quantified in three FAP expression scenarios. First, the A549 lung adenocarcinoma cell line was used to test for untargeted, FAP-independent activity in a FAP-negative setting. Next, A549 cells engineered for ectopic FAP expression were used to characterize FAP-dependent activity in cis, while both FAP-positive and FAP-negative cell lines were differentially labelled and co-cultured to compare cis and trans activity of FAP-dependent IFNγ mimetics. Split dual-targeted IFNγ mimetics were serially diluted to span a concentration range from 10 nM to 1 pM. Recombinant IFNγ and the intact, parental VHH pair deficient in FAP targeting (P1AH1884, format depicted in FIG. 5) were used as reference molecules. Following a 72 h incubation in the three FAP expression scenarios, MHC-I and PD-L1 expression levels were quantified by flow cytometry. In the absence of FAP expression, MHC-I and PD-L1 upregulation was induced by reference molecules while none of the combinations of split dual-targeted molecules exhibited FAP-independent activity (FIGS. 12A and 12B). In the presence of FAP expression, both the recapitulation of a cis setting (FIGS. 12C and 12D) and the recapitulation of a trans setting (FIGS. 12E and 12F) showed FAP-specific activity. In particular, the cis setting underscored the importance of geometry to achieve potent activity, with minor format adjustments (i.e. swapping of fusion points from VH to VL on anti-FAP binders) translating into major differences in IFNγR signaling. The P1AI0831+P1AI0066 pair provided the most potent FAP-dependent IFNγR activity. Interestingly, the geometrical constraints did not apply to the same degree in a trans scenario, with all format combinations displaying FAP-dependent activity.

To further explore format options and extend our understanding of the constraints imposed by molecular geometry, we designed additional sets of molecules in which the VHHs were further distanced from the FAP binding domains. The VHHs were fused to the N-terminus of the opposite Fc chain via a 3 (G₄S) linker (FIG. 13A-D) or to the C-terminus of the same Fc chain via a 3 (G₄S) linker (FIG. 13E-H). The sets were tested in vitro in the three FAP expression scenarios, as described previously. FAP-independent activity was observed with the reference molecules (recombinant IFNγ and P1AH1884, FIGS. 14A and 14B) while split dual-targeted combinations did not show IFNγR agonism except at high concentration for one combination (P1AI5306+P1AI5307: FIG. 14A). Next, FAP-dependent activity was tested in cells expressing FAP (cis targeting). MHC-I upregulation (FIG. 14C) and PD-L1 upregulation (FIG. 14D) was observed with the reference molecules and the P1AI5306+P1AI5307 combination, but all other combinations remained inactive. Finally, the combinations were tested in a trans scenario, to assess activity on FAP-negative cells in the presence of FAP-expressing cells. All combinations showed FAP-dependent trans activity, inducing both MHC-1 (FIG. 14E) and PD-L1 upregulation (FIG. 14F). By disconnecting the VHHs from the FAP binding moiety, FAP-dependent cis activity was rarely observed due to the previously identified geometrical constraints imposed in a cis scenario. However, in a trans setting, all combinations displayed IFNγR agonism regardless of the format.

### 3.2 Design of PD1-Dependent Biparatopic Assembly of IL-2 Mimetics

In a bid to maintain conditional agonistic activity whilst incorporating tumor selectivity, a strategy was devised to harness cell surface markers, reportedly enriched within the tumor environment, to scaffold the assembly of the IL-2 agonistic VHH domain pair. The aim was to bind two proximal epitopes on a single target using two non-competing anti-target binders (FIG. 15A). In the first instance, PD-I was selected as the target for several reasons. First, upregulation of PD-1 is observed upon T cell activation, providing an opportunity to enhance selectivity towards the tumor-specific effector T cell population. Second, PD-L1-competitive anti-PD-1 binders would provide immune checkpoint inhibition mediated by blockade of the PD1/PD-L1 signaling cascade. Third, recent evidence highlighted the synergistic effects of IL-2R agonism combined with cis-targeted PD-1 blockade, resulting in the differentiation of ‘better effector’ T cells (Codarri Deak et al. Nature 610:161-172 (2022)).

In this experiment, the potency and the cis-delivery of two pairs of IL-2 mimetics (FIGS. 15B and 15C), as well as PD1-IL2v (FIG. 15E, P1AE4422) and a potent IL-2 agonistic heavy chain antibody (FIG. 15D, P1AH117) as reference molecules, were measured based on their IL-2R signaling by treating activated PD-1′ and PD-1 (anti-PD1 pre-treated) CD4 T cells with increasing concentrations of molecules. The purpose was to determine the dependency of the PD-1-based IL-2R agonists on the PD1 expression of the T cells in order to deliver an IL-2R signaling.

For this purpose CD4 T cells were sorted from healthy donor PBMCs with CD4 beads (, Miltenyi, Cat. No: 130-045-101) and activated for 3 days in presence of 1 μg/ml plate bound anti-CD3 (overnight pre-coated, clone OKT3, Cat. No: 317315, BioLegend) and 1 μg/ml of soluble anti-CD28 (clone CD28.2, Cat. No: 302923, BioLegend) antibodies to induce PD1 expression. Three days later, the cells were harvested and washed several times to remove endogenous cytokines and half of the cells were labelled with CTV (5 μM, 5 min at 20° C.: Cat. No: C34557. Thermo Scientific) and the other half left unlabelled.

Then, the unabelled cells were incubated with a saturating concentration of a competing anti-PD-1 antibody (in-house molecule, 10 μg/ml) for 30 min at 20° C. followed by several washing steps to remove the excess unbound anti-PD1 antibody. Thereafter the PD1 pre-blocked unlabelled cells (25 μl, 6×10⁶ cells/ml) were co-cultured 1:1 with the PD1⁺ CTV labelled cells (25 μl, 6×10⁶ cells/ml) in a V-bottom plate before being treated for 15 or 60 min at 37° C. with increasing concentrations of treatment molecules (50 μl, 1:10 dilution steps). To preserve the phosphorylation state, an equal amount of Phosphoflow Fix Buffer I (100 μl, Cat. No: 557870, BD) was added after incubation with the various treatment molecules. The cells were then incubated for an additional 30 min at 37° C. before being permeabilized overnight at −80° C. with Phosphoflow PermBuffer III (Cat. No: 558050, BD). On the next day STAT-5 in its phosphorylated form was stained for 30 min at 4° C. by using an anti-STAT-5P antibody (47/Stat5 (pY694) clone. Cat. No: 562076, BD).

The cells were acquired at the FACS BD-LSR Fortessa (BD Bioscience). The frequency of STAT-5P was determined with FlowJo (V10) and plotted with GraphPad Prism.

The dose-response curves on PD-1⁺ T cells provide information on the potency of the PD1-based IL-2R agonists in signaling through the IL-2R. In addition, the dose-response curves on T cells pre-treated with a competing anti-PD1 antibody, to prevent the PD1 mediated delivery, show the potency of the PD1-based IL-2R agonist molecules in providing IL-2R signaling independently from PD-1 expression. PD1-IL2v showed reduced activity on T cells in absence of PD1 binding (pre-blocked) (FIG. 16, black solid and black dashed lines). While no activity was seen for the two combinations of split IL-2 mimetics (P1AH6850+P1AH6813, P1AH6814+P1AI1593) in absence of PD1 binding (FIG. 16, light grey dashed lines). No reduction in IL-2 signaling was seen for the non-targeted IL-2 agonistic bispecific heavy chain antibody (P1AH1177, FIG. 16, dark gray solid and dark grey dashed lines).

Further investigations of the biparatopic assembly concept were undertaken with alternative anti-PD-1 Fab binders (FIG. 17). HEK Blue IL-2 wt cells (PD-1-negative) and HEK Blue IL-2 cells expressing human PD-1 were used to assess IL-2 signaling.

As observed with the previously described PD-1-targeted IL-2R agonists (P1AH6814+P1AI1593 and P1AH6850+P1AH6813), none of the tested combinations showed IL-2R signaling in the absence of PD-1 expression (FIG. 18A) whereas replacing PD-1 binder 0376 with 7G12 (P1AK2802+P1AK2599) also demonstrated strong potency on PD-1 expressing cells (FIG. 18B). Of the eight tested PD-1-targeted IL-2R agonists containing the same set of PD-1 binders (7G12 and 1040), three demonstrated strong dose-response IL-2R activation.

### 3.3 FAP-Dependent Biparatopic Assembly of IFNγ Mimetics Using a Single Molecule Format

To ascertain whether a single molecule retaining all features of the split bimolecular approach could be achieved, we designed a tetraspecific format incorporating the requirements of the split dual-targeted molecules. The all-in-one format provides biparatopic assembly of a cytokine receptor activating VHH domain pair by amalgamating two functional split biparatopic molecules into one molecule. First, the most potent split configuration of IFNγ agonists was selected, which was shown to provide both cis and trans activity (P1AI0831+P1AI0066; FIG. 12). Next, the two selected VHH-Fab fusions were grafted onto a single molecule, harnessing the Fc as a spacer in a bid to distance the VHH domains and reduce the likelihood VHH-mediated IFNγR activation in a FAP-independent manner. Finally, the knob-into-hole and CrossFab technologies were incorporated to generate the tetraspecific format, all whilst preserving the optimal fusion configuration defined in the P1AI0831+P1AI0066 combination (FIG. 19). In detail, the VHH domain with IFNγR1 specificity was fused to the N-terminus of the anti-FAP binder 1 VL domain using a 5 (G₄S) linker. The N-terminus of the anti-FAP binder 1 VH domain was fused to the C-terminus of the Fc knob chain via a 3 (G₄S) linker and the CH1/CL domains were modified to contain additional attractive charges. The anti-FAP binder 2 was located upstream of the hinge on the hole chain and contained crossed VH/VL domains. The VHH domain with IFNγR2 specificity was fused to the N-terminus of the VH domain, now located on the light chain, using a 5 (G₄S) linker.

The resulting all-in-one format, referred to as P1AI5012, was tested in vitro for FAP-specific IFNγR activation using the three FAP expression scenarios previously described. In the absence of FAP expression, reference molecules showed MHC-I upregulation (FIG. 20A, dashed lines) and PD-L1 upregulation (FIG. 20B, dashed lines), while P1AI5012 did not induce IFNγR activation (FIGS. 20A and 20B, solid line). On cells expressing FAP, P1AI5012 displayed FAP-dependent IFNγR agonism, exhibiting activity similar to recombinant IFNγ at low concentrations of 25 pM (FIGS. 20C and 20D). In a setting recapitulating trans activity, potent IFNγR agonism was observed in the presence of P1AI5012 (FIGS. 20E and 20F).

3.4 Additional Formats of “all-In-One” Molecules

To ascertain whether the mechanism of action is intrinsic to the specific format generated, we designed several variants of the all-in-one format (P1AI5012) which differ in agonistic VHH pair (VHH pair 1: IFNγR1_3 VHH and IFNγR2_2 derived from P1AH1884; VHH pair 2: IFNγR1_5 VHH and IFNγR2_1 VHH derived from P1AH1886), the position of the VHHs, respectively on the VH or VL of the Fab domains, different position for the FAP binders, and the linker length between the Fab domains and the VHHs (5, 10, 15, and 25 amino acid) linker length (FIG. 21). P1AI5012, P1AJ0690, P1AJ0672, P1AJ0700 and P1AJ0685 comprise the VHH pair 1. P1AJ0744, P1AJ0735 and P1AJ0747 comprise the VHH pair 2.

The resulting all-in-one formats were tested in vitro for FAP-specific IFNγR activation using the three FAP expression scenarios previously described.

Based on the MHC-I up-regulation, tested all-in-one variants can be divided in three different groups. In detail, variants that are able to induce high levels of MHC-I. To this group belong the rec. IFNγ, the reference molecule (P1AI5012), and the P1AJ0690 all-in-one variant. Variants able to induce moderate MHC-I up-regulation. To this group belong the P1AJ0672, P1AJ0685, P1AJ0744 and the P1AJ0735 all-in-one variants. Lastly, variants able to induce low/none MHC-I up-regulation. To this group belong the following variants: P1AJ0700, and P1AJ0747 (FIG. 22).

### 3.5 PD-1-Dependent Biparatopic Assembly of IL-2 Mimetics Using a Single Molecule Format

To ascertain whether a single molecule retaining all features of the split IL-2 mimetic approach could be achieved, we designed a tetraspecific format incorporating the requirements of the split dual-targeted molecules (FIG. 23). The all-in-one format provided biparatopic assembly of a cytokine mimetic VHH pair by amalgamating two functional split biparatopic molecules into one molecule. First, the most potent split configuration of IL-2 mimetics was selected, which was shown to elicit PD1-mediated cis activity (P1AI1593 and P1AH6814). Next, the two selected VHH-Fab fusions were grafted onto a single molecule, harnessing the Fc as a spacer in a bid to distance the VHH domains and reduce the likelihood of VHH-mediated IL-2R activation in a PD1-independent manner. Finally, the knob-into hole and CrossFab technologies were incorporated to generate the tetraspecific format, all whilst preserving the optimal fusion configuration defined in the P1AI1593+P1AH6814 combination. In detail, the VHH domain with IL-2Rβ specificity was fused to the N-terminus of the anti-PD1 binder 1 VL domain using a 5 (G4S) linker. The N-terminus of the anti-PD1 binder 1 VH domain was fused to the C-terminus of the Fc knob chain via a 2 (G4S) linker and the CH1/CL domains were modified to contain additional attractive charges. The anti-PD1 binder 2 was located upstream of the hinge on the hole chain and contained crossed VH/VL domains. The VHH domain with IL-2Rγ specificity was fused to the N-terminus of the VH domain, now located on the light chain, using a 5 (G4S) linker.

To measure the capacity of PD-1 targeted IL-2R agonists to induce IL-2R signaling, HEK Blue IL-2 cells (Invivogen) with or without human PD-1 expression were used. The reporter system measures secreted SEAP levels by absorbance readout as a proxy for STAT-5 phosphorylation. Briefly, 12,000 cells per well were seeded in a 384-well flat bottom tissue culture treated plate (Corning, Cat. No. 3701). Test compounds (4-fold dilution series, starting concentration of 100) nM) were added to the assay plate. Samples were incubated at 37° C. and 5% CO₂ for 20 h before assay readout with QUANTI-Blue solution (Invivogen, Cat. No. rep-qbs3) according to the manufacturer's instructions. Absorbance readout was performed using Tecan Spark 10M plate reader. Dose-response of test compounds versus IL2R signaling (SEAP levels at A650) were plotted in GraphPad Prism software. While Fc-fused IL2R agonists showed activity in the presence and absence of PD-1, the capacity of the all-in-one PD-1 targeted IL-2R agonist (P1AI5057) was strongly dependent on PD-1 (FIG. 24).

3.6. Functional Characterization of Monoparatopic and Biparatopic Cis-Targeted IL-2R all-In-One Agonists

To understand the requirement for biparatopic assembly of IL-2R agonists, we tested the monoparatopic concept of IL-2R signaling via binding to the same PD-1 epitope (FIG. 25). HEK Blue IL-2 wt and HEK Blue IL-2 human PD-1 cells were used to assess IL-2 signaling. As previously demonstrated, biparatopic assembly of PD-1-targeted IL-2R agonists (P1AI5057) showed strong dose-response IL-2R signaling in the reporter assay (FIG. 26B), whereas no preferential PD-1 dependent activity was observed with the two monoparatopic compounds tested (FIGS. 26A and 26B), highlighting the biparatopic assembly requirement for functional IL-2R signalling. Of note, the residual PD-1 independent activity of PD-1 targeted IL-2R agonists was similar with monoparatopic and biparatopic molecules (FIG. 26A).

The in vitro data provided robust evidence that IFNγ and IL-2 mimetics can be engineered for conditional activity under the desired settings using biparatopic molecular assembly strategies. As a first approach, it has been demonstrated that VHH pairs mimicking IFNγ and IL-2 could be rendered non-functional by distribution onto two separate molecules and the assembly of a functional IFNγ and IL-2 mimetics could be mediated under the desired conditions. As an extension to this approach, the possibility to design a single molecule format retaining conditional cis and trans activity was explored. The all-in-one format was achieved through distancing of the VHH pair to inactivate the cytokine mimetic, whilst adhering to molecular configurations to fulfill the geometrical requirements of conditional cis and trans agonistic activity. It has been demonstrated that format and geometry can be harnessed to fine-tune the activity of conditional cytokine mimetics.

Sequences

| | SEQ ID | |
|---|---|---|
| Description | NO | Sequence |
| IFNgR1_1 VHH | 1 | QVQLQESGGGLVQAGGSLRLSCVASGSLFRWTAMAWY |
| | | RQAPGKQRELVAVISPAGSIDYADSVKGRFTISRDNGKN |
| | | VVYLQMNSLKPEDTAVYYCSIHHSARAYWGQGTQVTV |
| | | SS |
| IFNgR1_2 VHH | 2 | QVQLQESGGGLVQPGGSLTLSCAASIHLFSRTAMDWYR |
| | | QAPGRQRELVATISPVGSTYYADSAKDRFTISRDNANNM |
| | | VYLQMNSLGPEDTAVYYCISGQLQGQGTQVTVSS |
| IFNgR1_3 VHH | 3 | QVQLQQSGGGLVQAGGSLRLSCTASGSTFSFSNYHMGW |
| | | YRQAPGKQRERVASISSRDSTYYSDSVKGRFTISRDTAR |
| | | NTVYLQMNSLEPEETAVYYCNARGRATGRDYWGQGTQ |
| | | VTVSS |
| IFNgR1_4 VHH | 4 | QVQLQQFGGGLVQAGGSLRLSCTASGSTFSFSNYHMGW |
| | | YRQAPGKQRERVASISSRGSTYYSDSVKGRFAISRDTAR |
| | | NTVYLQMNSLEPEETAVYYCNARGRATGRDYWGQGTL |
| | | VTVSS |
| IFNgR1_5 VHH | 5 | QVQLQQFGGGLVQPGGSLRLSCAVSRSIFSIDAMAWYR |
| | | QAPGKQRNLVGTITSDGTTNYVDSAKGRFTISRDNAKNT |
| | | VYLQMNSLKPEDTAVYFCNAGAVSRTGGYRPSGYWGQ |
| | | GTLVTVSS |
| IFNgR2_1 VHH | 6 | QVQLQESGGGLVQAGGSLRLSCTASGAYFIMGWFRQAP |
| | | GKQRELVARTIRDGTTDYADSVKGRFTISRNTAENTAYL |
| | | QMNSLKPEDTGVYYCAAGPLSKSFAPWDPAYWGQGTL |
| | | VTVSS |
| IFNgR2_2 VHH | 7 | QVQLQEFGGGLVQAGEALRLSCVASKTIFNTMPMGWY |
| | | RQAPGKERELVATITSSGVVNSADSVKGRFTISRDSAKR |
| | | TAYLQMNNLKPEDTAVYYCAAMFKPGIPEYWGRGTQV |
| | | TVSS |
| IFNgR2_3 VHH | 8 | QVQLQQFGGGLVQPGGSLTLSCAASGSIFSINPMGWYRQ |
| | | APGKRRELVASLTNRGITTYADSVKGRFTISRDNAKNTV |
| | | YLQMNALKPEDTAVYYCHAYVDTGSHEYPQYENYWG |
| | | QGTLVTVSS |
| IFNgR2_4 VHH | 9 | QVQLQESGGGLVQAGGSLRLSCAASGSTFTFSTMAWYR |
| | | QAPGKQRELVATFFSPSTWYADSVEGRFTVSRNNAKNT |
| | | MYLHMNSLKPEDTAVYYCTSPSASNGQGTQVTVSS |
| IFNgR2_5 VHH | 10 | QVQLQESGGGLVQPGGSLTLSCAASGSIVSINPMGWYR |
| | | QAPGKRRELVASLTNRGITTYADSVKGRFTISRDNAKNT |
| | | VYLQMNTLKPEDTAVYYCHAYVDTGNYEYPQYENYW |
| | | GQGTLVTVSS |
| IFNgR1_1 heavy | 11 | QVQLQESGGGLVQAGGSLRLSCVASGSLFRWTAMAWY |
| chain antibody (knob | | RQAPGKQRELVAVISPAGSIDYADSVKGRFTISRDNGKN |
| chain) | | VVYLQMNSLKPEDTAVYYCSIHHSARAYWGQGTQVTV |
| | | SSTSGSGGGGSGGGGSGGGGSGGGGSGGGGDKTHTCPP |
| | | CPAPEAAGGPSVFLFPPKPKDTLMISRTPEVTCVVVDVS |
| | | HEDPEVKFNWYVDGVEVHNAKTKPREEQYNSTYRVVS |
| | | VLTVLHQDWLNGKEYKCKVSNKALGAPIEKTISKAKGQ |
| | | PREPQVYTLPPCRDELTKNQVSLWCLVKGFYPSDIAVE |
| | | WESNGQPENNYKTTPPVLDSDGSFFLYSKLTVDKSRWQ |
| | | QGNVFSCSVMHEALHNHYTQKSLSLSPG |
| IFNgR1_2 heavy | 12 | QVQLQESGGGLVQPGGSLTLSCAASIHLFSRTAMDWYR |
| chain antibody (knob | | QAPGRQRELVATISPVGSTYYADSAKDRFTISRDNANNM |
| chain) | | VYLQMNSLGPEDTAVYYCISGQLQGQGTQVTVSSTSGS |
| | | GGGGSGGGGSGGGGSGGGGSGGGGDKTHTCPPCPAPE |
| | | AAGGPSVFLFPPKPKDTLMISRTPEVTCVVVDVSHEDPE |
| | | VKFNWYVDGVEVHNAKTKPREEQYNSTYRVVSVLTVL |
| | | HQDWLNGKEYKCKVSNKALGAPIEKTISKAKGQPREPQ |
| | | VYTLPPCRDELTKNQVSLWCLVKGFYPSDIAVEWESNG |
| | | QPENNYKTTPPVLDSDGSFFLYSKLTVDKSRWQQGNVF |
| | | SCSVMHEALHNHYTQKSLSLSPG |
| IFNg_R1_3 heavy | 13 | QVQLQQSGGGLVQAGGSLRLSCTASGSTFSFSNYHMGW |
| chain antibody (knob | | YRQAPGKQRERVASISSRDSTYYSDSVKGRFTISRDTAR |
| chain) | | NTVYLQMNSLEPEETAVYYCNARGRATGRDYWGQGTQ |
| | | VTVSSTSGSGGGGSGGGGSGGGGSGGGGSGGGGDKTH |
| | | TCPPCPAPEAAGGPSVFLFPPKPKDTLMISRTPEVTCVVV |
| | | DVSHEDPEVKFNWYVDGVEVHNAKTKPREEQYNSTYR |
| | | VVSVLTVLHQDWLNGKEYKCKVSNKALGAPIEKTISKA |
| | | KGQPREPQVYTLPPCRDELTKNQVSLWCLVKGFYPSDIA |
| | | VEWESNGQPENNYKTTPPVLDSDGSFFLYSKLTVDKSR |
| | | WQQGNVFSCSVMHEALHNHYTQKSLSLSPG |
| IFNgR1_5 heavy | 14 | QVQLQQFGGGLVQPGGSLRLSCAVSRSIFSIDAMAWYR |
| chain antibody (knob | | QAPGKQRNLVGTITSDGTTNYVDSAKGRFTISRDNAKNT |
| chain) | | VYLQMNSLKPEDTAVYFCNAGAVSRTGGYRPSGYWGQ |
| | | GTLVTVSSTSGSGGGGSGGGGSGGGGGDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLT |
| | | VLHQDWLNGKEYKCKVSNKALGAPIEKTISKAKGQPRE |
| | | PQVYTLPPCRDELTKNQVSLWCLVKGFYPSDIAVEWES |
| | | NGQPENNYKTTPPVLDSDGSFFLYSKLTVDKSRWQQGN |
| | | VFSCSVMHEALHNHYTQKSLSLSPG |
| IFNgR2_1 heavy | 15 | QVQLQESGGGLVQAGGSLRLSCTASGAYFIMGWFRQAP |
| chain antibody (hole | | GKQRELVARTIRDGTTDYADSVKGRFTISRNTAENTAYL |
| chain) | | QMNSLKPEDTGVYYCAAGPLSKSFAPWDPAYWGQGTL |
| | | VTVSSTSGSGGGGSGGGGSGGGGSGGGGSGGGGDKTH |
| | | TCPPCPAPEAAGGPSVFLFPPKPKDTLMISRTPEVTCVVV |
| | | DVSHEDPEVKFNWYVDGVEVHNAKTKPREEQYNSTYR |
| | | VVSVLTVLHQDWLNGKEYKCKVSNKALGAPIEKTISKA |
| | | KGQPREPQVCTLPPSRDELTKNQVSLSCAVKGFYPSDIA |
| | | VEWESNGQPENNYKTTPPVLDSDGSFFLVSKLTVDKSR |
| | | WQQGNVFSCSVMHEALHNHYTQKSLSLSPG |
| IFNgR2_2 heavy | 16 | QVQLQEFGGGLVQAGEALRLSCVASKTIFNTMPMGWY |
| chain antibody (hole | | RQAPGKERELVATITSSGVVNSADSVKGRFTISRDSAKR |
| chain) | | TAYLQMNNLKPEDTAVYYCAAMFKPGIPEYWGRGTQV |
| | | TVSSTSGSGGGGSGGGGSGGGGSGGGGSGGGGDKTHTC |
| | | PPCPAPEAAGGPSVFLFPPKPKDTLMISRTPEVTCVVVDV |
| | | SHEDPEVKFNWYVDGVEVHNAKTKPREEQYNSTYRVV |
| | | SVLTVLHQDWLNGKEYKCKVSNKALGAPIEKTISKAKG |
| | | QPREPQVCTLPPSRDELTKNQVSLSCAVKGFYPSDIAVE |
| | | WESNGQPENNYKTTPPVLDSDGSFFLVSKLTVDKSRWQ |
| | | QGNVFSCSVMHEALHNHYTQKSLSLSPG |
| IFNgR2_3 heavy | 17 | QVQLQQFGGGLVQPGGSLTLSCAASGSIFSINPMGWYRQ |
| chain antibody (hole | | APGKRRELVASLTNRGITTYADSVKGRFTISRDNAKNTV |
| chain) | | YLQMNALKPEDTAVYYCHAYVDTGSHEYPQYENYWG |
| | | QGTLVTVSSTSGSGGGGSGGGGSGGGGSGGGGSGGGG |
| | | DKTHTCPPCPAPEAAGGPSVFLFPPKPKDTLMISRTPEVT |
| | | CVVVDVSHEDPEVKFNWYVDGVEVHNAKTKPREEQYN |
| | | STYRVVSVLTVLHQDWLNGKEYKCKVSNKALGAPIEKT |
| | | ISKAKGQPREPQVCTLPPSRDELTKNQVSLSCAVKGFYP |
| | | SDIAVEWESNGQPENNYKTTPPVLDSDGSFFLVSKLTVD |
| | | KSRWQQGNVFSCSVMHEALHNHYTQKSLSLSPG |
| IFNgR2_4 heavy | 18 | QVQLQESGGGLVQAGGSLRLSCAASGSTFTFSTMAWYR |
| chain antibody (hole | | QAPGKQRELVATFFSPSTWYADSVEGRFTVSRNNAKNT |
| chain) | | MYLHMNSLKPEDTAVYYCTSPSASNGQGTQVTVSSTSG |
| | | SGGGGSGGGGSGGGGSGGGGSGGGGDKTHTCPPCPAPE |
| | | AAGGPSVFLFPPKPKDTLMISRTPEVTCVVVDVSHEDPE |
| | | VKFNWYVDGVEVHNAKTKPREEQYNSTYRVVSVLTVL |
| | | HQDWLNGKEYKCKVSNKALGAPIEKTISKAKGQPREPQ |
| | | VCTLPPSRDELTKNQVSLSCAVKGFYPSDIAVEWESNGQ |
| | | PENNYKTTPPVLDSDGSFFLVSKLTVDKSRWQQGNVFS |
| | | CSVMHEALHNHYTQKSLSLSPG |
| IFNgR2_5 heavy | 19 | QVQLQESGGGLVQPGGSLTLSCAASGSIVSINPMGWYR |
| chain antibody (hole | | QAPGKRRELVASLTNRGITTYADSVKGRFTISRDNAKNT |
| chain) | | VYLQMNTLKPEDTAVYYCHAYVDTGNYEYPQYENYW |
| | | GQGTLVTVSSTSGSGGGGSGGGGSGGGGSGGGGSGGG |
| | | GDKTHTCPPCPAPEAAGGPSVFLFPPKPKDTLMISRTPEV |
| | | TCVVVDVSHEDPEVKFNWYVDGVEVHNAKTKPREEQY |
| | | NSTYRVVSVLTVLHQDWLNGKEYKCKVSNKALGAPIEK |
| | | TISKAKGQPREPQVCTLPPSRDELTKNQVSLSCAVKGFY |
| | | PSDIAVEWESNGQPENNYKTTPPVLDSDGSFFLVSKLTV |
| | | DKSRWQQGNVFSCSVMHEALHNHYTQKSLSLSPG |
| FAP 4B9 VH | 20 | EVQLLESGGGLVQPGGSLRLSCAASGFTFSSYAMSWVR |
| | | QAPGKGLEWVSAIIGSGASTYYADSVKGRFTISRDNSKN |
| | | TLYLQMNSLRAEDTAVYYCAKGWFGGFNYWGQGTLV |
| | | TVSS |
| FAP 4B9 VL | 21 | EIVLTQSPGTLSLSPGERATLSCRASQSVTSSYLAWYQQ |
| | | KPGQAPRLLINVGSRRATGIPDRFSGSGSGTDFTLTISRLE |
| | | PEDFAVYYCQQGIMLPPTFGQGTKVEIK |
| FAP clone 1G1a | 22 | QVQLVQSGAEVKKPGASVKVSCKASGYTLTDYNMDW |
| (humanized 212) VH | | VRQAPGQGLEWIGDIYPNTGGTIYNQKFKGRVTMTIDTS |
| | | TSTVYMELSSLRSEDTAVYYCTRFRGIHYAMDYWGQGT |
| | | TVTVSS |
| FAP clone 1G1a | 23 | EIVLTQSPATLSLSPGERATLSCRASESVDNYGLSFINWF |
| (humanized 212) VL | | QQKPGQAPRLLIYGTSNRGSGIPARFSGSGSGTDFTLTISS |
| | | LEPEDFAVYFCQQSNEVPYTFGGGTKVEIK |
| Reference molecule | 24 | EVQLLESGGGLVQPGGSLRLSCAASGFTFSSYAMSWVR |
| FAP 4B9 Fab-Fc PG | | QAPGKGLEWVSAIIGSGASTYYADSVKGRFTISRDNSKN |
| LALA-human IFNg | | TLYLQMNSLRAEDTAVYYCAKGWFGGFNYWGQGTLV |
| 1-132 S132P | | TVSSASTKGPSVFPLAPSSKSTSGGTAALGCLVKDYFPEP |
| (P1AF3574) HC | | VTVSWNSGALTSGVHTFPAVLQSSGLYSLSSVVTVPSSS |
| | | LGTQTYICNVNHKPSNTKVDKKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLT |
| | | VLHQDWLNGKEYKCKVSNKALGAPIEKTISKAKGQPRE |
| | | PQVYTLPPSRDELTKNQVSLTCLVKGFYPSDIAVEWESN |
| | | GQPENNYKTTPPVLDSDGSFFLYSKLTVDKSRWQQGNV |
| | | FSCSVMHEALHNHYTQKSLSLSPGGGGGSGGGGSGGGG |
| | | SQDPYVKEAENLKKYFNAGHSDVADNGTLFLGILKNW |
| | | KEESDRKIMQSQIVSFYFKLFKNFKDDQSIQKSVETIKED |
| | | MNVKFFNSNKKKRDDFEKLTNYSVTDLNVQRKAIHELI |
| | | QVMAELSPAAKTGKRKRP |
| FAP 4B9 Fab LC | 25 | EIVLTQSPGTLSLSPGERATLSCRASQSVTSSYLAWYQQ |
| (P1AF3574, | | KPGQAPRLLINVGSRRATGIPDRFSGSGSGTDFTLTISRLE |
| P1AI0160, P1AI4711, | | PEDFAVYYCQQGIMLPPTFGQGTKVEIKRTVAAPSVFIFP |
| P1AI0183, P1AI5305) | | PSDEQLKSGTASVVCLLNNFYPREAKVQWKVDNALQSG |
| | | NSQESVTEQDSKDSTYSLSSTLTLSKADYEKHKVYACEV |
| | | THQGLSSPVTKSFNRGEC |
| Recombinant human | 26 | MQDPYVKEAENLKKYFNAGHSDVADNGTLFLGILKNW |
| IFNg (Peprotech) | | KEESDRKIMQSQIVSFYFKLFKNFKDDQSIQKSVETIKED |
| | | MNVKFFNSNKKKRDDFEKLTNYSVTDLNVQRKAIHELI |
| | | QVMAELSPAAKTGKRKRSQMLFQGRRASQ |
| P1AI0160 knob chain | 27 | QVQLQQSGGGLVQAGGSLRLSCTASGSTFSFSNYHMGW |
| | | YRQAPGKQRERVASISSRDSTYYSDSVKGRFTISRDTAR |
| | | NTVYLQMNSLEPEETAVYYCNARGRATGRDYWGQGTQ |
| | | VTVSSGGSGGGGSGGGGSGGGGSGGGGSGGEVQLLESG |
| | | GGLVQPGGSLRLSCAASGFTFSSYAMSWVRQAPGKGLE |
| | | WVSAIIGSGASTYYADSVKGRFTISRDNSKNTLYLQMNS |
| | | LRAEDTAVYYCAKGWFGGFNYWGQGTLVTVSSASTKG |
| | | PSVFPLAPSSKSTSGGTAALGCLVKDYFPEPVTVSWNSG |
| | | ALTSGVHTFPAVLQSSGLYSLSSVVTVPSSSLGTQTYICN |
| | | VNHKPSNTKVDKKVEPKSCDKTHTCPPCPAPEAAGGPS |
| | | VFLFPPKPKDTLMISRTPEVTCVVVDVSHEDPEVKFNWY |
| | | VDGVEVHNAKTKPREEQYNSTYRVVSVLTVLHQDWLN |
| | | GKEYKCKVSNKALGAPIEKTISKAKGQPREPQVYTLPPC |
| | | RDELTKNQVSLWCLVKGFYPSDIAVEWESNGQPENNYK |
| | | TTPPVLDSDGSFFLYSKLTVDKSRWQQGNVFSCSVMHE |
| | | ALHNHYTQKSLSLSPG |
| Fc hole chain | 28 | DKTHTCPPCPAPEAAGGPSVFLFPPKPKDTLMISRTPEVT |
| (P1AI0160, | | CVVVDVSHEDPEVKFNWYVDGVEVHNAKTKPREEQYN |
| P1AI0066, P1AI0831, | | STYRVVSVLTVLHQDWLNGKEYKCKVSNKALGAPIEKT |
| P1AI0832, P1AI4708, | | ISKAKGQPREPQVCTLPPSRDELTKNQVSLSCAVKGFYP |
| P1AI4709, P1AI4710, | | SDIAVEWESNGQPENNYKTTPPVLDSDGSFFLVSKLTVD |
| P1AI4711, P1AI0187, | | KSRWQQGNVFSCSVMHEALHNHYTQKSLSLSPG |
| P1AI5306, | | |
| P1AH6850, | | |
| P1AH6814, | | |
| P1AK2802, | | |
| P1AK2803, | | |
| P1AK2809, | | |
| P1AK2810, | | |
| P1AK2599, | | |
| P1AK2799, | | |
| P1AK2806, | | |
| P1AK2798) | | |
| LC (P1AI0066, | 29 | EIVLTQSPATLSLSPGERATLSCRASESVDNYGLSFINWF |
| P1AI4710, P1AI0184, | | QQKPGQAPRLLIYGTSNRGSGIPARFSGSGSGTDFTLTISS |
| P1AI5304) | | LEPEDFAVYFCQQSNEVPYTFGGGTKVEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |
| P1AI0066 knob chain | 30 | QVQLQEFGGGLVQAGEALRLSCVASKTIFNTMPMGWY |
| | | RQAPGKERELVATITSSGVVNSADSVKGRFTISRDSAKR |
| | | TAYLQMNNLKPEDTAVYYCAAMFKPGIPEYWGRGTQV |
| | | TVSSGGSGGGGSGGGGSGGGGSGGGGSGGQVQLVQSG |
| | | AEVKKPGASVKVSCKASGYTLTDYNMDWVRQAPGQGL |
| | | EWIGDIYPNTGGTIYNQKFKGRVTMTIDTSTSTVYMELS |
| | | SLRSEDTAVYYCTRFRGIHYAMDYWGQGTTVTVSSAST |
| | | KGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHTCPPCPAPEAAGG |
| | | PSVFLFPPKPKDTLMISRTPEVTCVVVDVSHEDPEVKFN |
| | | WYVDGVEVHNAKTKPREEQYNSTYRVVSVLTVLHQD |
| | | WLNGKEYKCKVSNKALGAPIEKTISKAKGQPREPQVYT |
| | | LPPCRDELTKNQVSLWCLVKGFYPSDIAVEWESNGQPE |
| | | NNYKTTPPVLDSDGSFFLYSKLTVDKSRWQQGNVFSCS |
| | | VMHEALHNHYTQKSLSLSPG |
| P1AI0831 LC | 31 | QVQLQQSGGGLVQAGGSLRLSCTASGSTFSFSNYHMGW |
| | | YRQAPGKQRERVASISSRDSTYYSDSVKGRFTISRDTAR |
| | | NTVYLQMNSLEPEETAVYYCNARGRATGRDYWGQGTQ |
| | | VTVSSGGSGGGGSGGGGSGGGGSGGGGSGGEIVLTQSP |
| | | GTLSLSPGERATLSCRASQSVTSSYLAWYQQKPGQAPRL |
| | | LINVGSRRATGIPDRFSGSGSGTDFTLTISRLEPEDFAVYY |
| | | CQQGIMLPPTFGQGTKVEIKRTVAAPSVFIFPPSDEQLKS |
| | | GTASVVCLLNNFYPREAKVQWKVDNALQSGNSQESVT |
| | | EQDSKDSTYSLSSTLTLSKADYEKHKVYACEVTHQGLSS |
| | | PVTKSFNRGEC |
| knob chain | 32 | EVQLLESGGGLVQPGGSLRLSCAASGFTFSSYAMSWVR |
| (P1AI0831, | | QAPGKGLEWVSAIIGSGASTYYADSVKGRFTISRDNSKN |
| P1AI4708, P1AI0183, | | TLYLQMNSLRAEDTAVYYCAKGWFGGFNYWGQGTLV |
| P1AI5305) | | TVSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLT |
| | | VLHQDWLNGKEYKCKVSNKALGAPIEKTISKAKGQPRE |
| | | PQVYTLPPCRDELTKNQVSLWCLVKGFYPSDIAVEWES |
| | | NGQPENNYKTTPPVLDSDGSFFLYSKLTVDKSRWQQGN |
| | | VFSCSVMHEALHNHYTQKSLSLSPG |
| P1AI0832 LC | 33 | QVQLQEFGGGLVQAGEALRLSCVASKTIFNTMPMGWY |
| | | RQAPGKERELVATITSSGVVNSADSVKGRFTISRDSAKR |
| | | TAYLQMNNLKPEDTAVYYCAAMFKPGIPEYWGRGTQV |
| | | TVSSGGSGGGGSGGGGSGGGGSGGGGSGGEIVLTQSPA |
| | | TLSLSPGERATLSCRASESVDNYGLSFINWFQQKPGQAP |
| | | RLLIYGTSNRGSGIPARFSGSGSGTDFTLTISSLEPEDFAV |
| | | YFCQQSNEVPYTFGGGTKVEIKRTVAAPSVFIFPPSDEQL |
| | | KSGTASVVCLLNNFYPREAKVQWKVDNALQSGNSQES |
| | | VTEQDSKDSTYSLSSTLTLSKADYEKHKVYACEVTHQG |
| | | LSSPVTKSFNRGEC |
| knob chain | 34 | QVQLVQSGAEVKKPGASVKVSCKASGYTLTDYNMDW |
| (P1AI0832, | | VRQAPGQGLEWIGDIYPNTGGTIYNQKFKGRVTMTIDTS |
| P1AI4709, P1AI0184, | | TSTVYMELSSLRSEDTAVYYCTRFRGIHYAMDYWGQGT |
| P1AI5304) | | TVTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKKVEPKSCDKTHTCPPC |
| | | PAPEAAGGPSVFLFPPKPKDTLMISRTPEVTCVVVDVSH |
| | | EDPEVKFNWYVDGVEVHNAKTKPREEQYNSTYRVVSV |
| | | LTVLHQDWLNGKEYKCKVSNKALGAPIEKTISKAKGQP |
| | | REPQVYTLPPCRDELTKNQVSLWCLVKGFYPSDIAVEW |
| | | ESNGQPENNYKTTPPVLDSDGSFFLYSKLTVDKSRWQQ |
| | | GNVFSCSVMHEALHNHYTQKSLSLSPG |
| P1AI4708 LC | 35 | QVQLQEFGGGLVQAGEALRLSCVASKTIFNTMPMGWY |
| | | RQAPGKERELVATITSSGVVNSADSVKGRFTISRDSAKR |
| | | TAYLQMNNLKPEDTAVYYCAAMFKPGIPEYWGRGTQV |
| | | TVSSGGSGGGGSGGGGSGGGGSGGGGSGGEIVLTQSPG |
| | | TLSLSPGERATLSCRASQSVTSSYLAWYQQKPGQAPRLL |
| | | INVGSRRATGIPDRFSGSGSGTDFTLTISRLEPEDFAVYYC |
| | | QQGIMLPPTFGQGTKVEIKRTVAAPSVFIFPPSDEQLKSG |
| | | TASVVCLLNNFYPREAKVQWKVDNALQSGNSQESVTE |
| | | QDSKDSTYSLSSTLTLSKADYEKHKVYACEVTHQGLSSP |
| | | VTKSFNRGEC |
| P1AI4709 LC | 36 | QVQLQQSGGGLVQAGGSLRLSCTASGSTFSFSNYHMGW |
| | | YRQAPGKQRERVASISSRDSTYYSDSVKGRFTISRDTAR |
| | | NTVYLQMNSLEPEETAVYYCNARGRATGRDYWGQGTQ |
| | | VTVSSGGSGGGGSGGGGSGGGGSGGGGSGGEIVLTQSP |
| | | ATLSLSPGERATLSCRASESVDNYGLSFINWFQQKPGQA |
| | | PRLLIYGTSNRGSGIPARFSGSGSGTDFTLTISSLEPEDFA |
| | | VYFCQQSNEVPYTFGGGTKVEIKRTVAAPSVFIFPPSDEQ |
| | | LKSGTASVVCLLNNFYPREAKVQWKVDNALQSGNSQES |
| | | VTEQDSKDSTYSLSSTLTLSKADYEKHKVYACEVTHQG |
| | | LSSPVTKSFNRGEC |
| P1AI4710 knob chain | 37 | QVQLQQSGGGLVQAGGSLRLSCTASGSTFSFSNYHMGW |
| | | YRQAPGKQRERVASISSRDSTYYSDSVKGRFTISRDTAR |
| | | NTVYLQMNSLEPEETAVYYCNARGRATGRDYWGQGTQ |
| | | VTVSSGGSGGGGSGGGGSGGGGSGGGGSGGQVQLVQS |
| | | GAEVKKPGASVKVSCKASGYTLTDYNMDWVRQAPGQ |
| | | GLEWIGDIYPNTGGTIYNQKFKGRVTMTIDTSTSTVYME |
| | | LSSLRSEDTAVYYCTRFRGIHYAMDYWGQGTTVTVSSA |
| | | STKGPSVFPLAPSSKSTSGGTAALGCLVKDYFPEPVTVS |
| | | WNSGALTSGVHTFPAVLQSSGLYSLSSVVTVPSSSLGTQ |
| | | TYICNVNHKPSNTKVDKKVEPKSCDKTHTCPPCPAPEAA |
| | | GGPSVFLFPPKPKDTLMISRTPEVTCVVVDVSHEDPEVK |
| | | FNWYVDGVEVHNAKTKPREEQYNSTYRVVSVLTVLHQ |
| | | DWLNGKEYKCKVSNKALGAPIEKTISKAKGQPREPQVY |
| | | TLPPCRDELTKNQVSLWCLVKGFYPSDIAVEWESNGQPE |
| | | NNYKTTPPVLDSDGSFFLYSKLTVDKSRWQQGNVFSCS |
| | | VMHEALHNHYTQKSLSLSPG |
| P1AI4711 knob chain | 38 | QVQLQEFGGGLVQAGEALRLSCVASKTIFNTMPMGWY |
| | | RQAPGKERELVATITSSGVVNSADSVKGRFTISRDSAKR |
| | | TAYLQMNNLKPEDTAVYYCAAMFKPGIPEYWGRGTQV |
| | | TVSSGGSGGGGSGGGGSGGGGSGGGGSGGEVQLLESGG |
| | | GLVQPGGSLRLSCAASGFTFSSYAMSWVRQAPGKGLEW |
| | | VSAIIGSGASTYYADSVKGRFTISRDNSKNTLYLQMNSL |
| | | RAEDTAVYYCAKGWFGGFNYWGQGTLVTVSSASTKGP |
| | | SVFPLAPSSKSTSGGTAALGCLVKDYFPEPVTVSWNSGA |
| | | LTSGVHTFPAVLQSSGLYSLSSVVTVPSSSLGTQTYICNV |
| | | NHKPSNTKVDKKVEPKSCDKTHTCPPCPAPEAAGGPSVF |
| | | LFPPKPKDTLMISRTPEVTCVVVDVSHEDPEVKFNWYV |
| | | DGVEVHNAKTKPREEQYNSTYRVVSVLTVLHQDWLNG |
| | | KEYKCKVSNKALGAPIEKTISKAKGQPREPQVYTLPPCR |
| | | DELTKNQVSLWCLVKGFYPSDIAVEWESNGQPENNYKT |
| | | TPPVLDSDGSFFLYSKLTVDKSRWQQGNVFSCSVMHEA |
| | | LHNHYTQKSLSLSPG |
| hole chain (P1AI0183, | 39 | QVQLQQSGGGLVQAGGSLRLSCTASGSTFSFSNYHMGW |
| P1AI5304) | | YRQAPGKQRERVASISSRDSTYYSDSVKGRFTISRDTAR |
| | | NTVYLQMNSLEPEETAVYYCNARGRATGRDYWGQGTQ |
| | | VTVSSGGSGGGGSGGGGSGGDKTHTCPPCPAPEAAGGP |
| | | SVFLFPPKPKDTLMISRTPEVTCVVVDVSHEDPEVKFNW |
| | | YVDGVEVHNAKTKPREEQYNSTYRVVSVLTVLHQDWL |
| | | NGKEYKCKVSNKALGAPIEKTISKAKGQPREPQVCTLPP |
| | | SRDELTKNQVSLSCAVKGFYPSDIAVEWESNGQPENNY |
| | | KTTPPVLDSDGSFFLVSKLTVDKSRWQQGNVFSCSVMH |
| | | EALHNHYTQKSLSLSPG |
| hole chain (P1AI0184, | 40 | QVQLQEFGGGLVQAGEALRLSCVASKTIFNTMPMGWY |
| P1AI5305) | | RQAPGKERELVATITSSGVVNSADSVKGRFTISRDSAKR |
| | | TAYLQMNNLKPEDTAVYYCAAMFKPGIPEYWGRGTQV |
| | | TVSSGGSGGGGSGGGGSGGDKTHTCPPCPAPEAAGGPS |
| | | VFLFPPKPKDTLMISRTPEVTCVVVDVSHEDPEVKFNWY |
| | | VDGVEVHNAKTKPREEQYNSTYRVVSVLTVLHQDWLN |
| | | GKEYKCKVSNKALGAPIEKTISKAKGQPREPQVCTLPPS |
| | | RDELTKNQVSLSCAVKGFYPSDIAVEWESNGQPENNYK |
| | | TTPPVLDSDGSFFLVSKLTVDKSRWQQGNVFSCSVMHE |
| | | ALHNHYTQKSLSLSPG |
| LC (P1AI0187, | 41 | EVQLLESGGGLVQPGGSLRLSCAASGFTFSSYAMSWVR |
| P1AI5306) | | QAPGKGLEWVSAIIGSGASTYYADSVKGRFTISRDNSKN |
| | | TLYLQMNSLRAEDTAVYYCAKGWFGGFNYWGQGTLV |
| | | TVSSASVAAPSVFIFPPSDEQLKSGTASVVCLLNNFYPRE |
| | | AKVQWKVDNALQSGNSQESVTEQDSKDSTYSLSSTLTL |
| | | SKADYEKHKVYACEVTHQGLSSPVTKSFNRGEC |
| P1AI0187 knob chain | 42 | EIVLTQSPGTLSLSPGERATLSCRASQSVTSSYLAWYQQ |
| | | KPGQAPRLLINVGSRRATGIPDRFSGSGSGTDFTLTISRLE |
| | | PEDFAVYYCQQGIMLPPTFGQGTKVEIKSSASTKGPSVFP |
| | | LAPSSKSTSGGTAALGCLVKDYFPEPVTVSWNSGALTSG |
| | | VHTFPAVLQSSGLYSLSSVVTVPSSSLGTQTYICNVNHKP |
| | | SNTKVDKKVEPKSCDKTHTCPPCPAPEAAGGPSVFLFPP |
| | | KPKDTLMISRTPEVTCVVVDVSHEDPEVKFNWYVDGVE |
| | | VHNAKTKPREEQYNSTYRVVSVLTVLHQDWLNGKEYK |
| | | CKVSNKALGAPIEKTISKAKGQPREPQVYTLPPCRDELT |
| | | KNQVSLWCLVKGFYPSDIAVEWESNGQPENNYKTTPPV |
| | | LDSDGSFFLYSKLTVDKSRWQQGNVFSCSVMHEALHNH |
| | | YTQKSLSLSPGGGSGGGGSGGGGSGGQVQLQQSGGGLV |
| | | QAGGSLRLSCTASGSTFSFSNYHMGWYRQAPGKQRERV |
| | | ASISSRDSTYYSDSVKGRFTISRDTARNTVYLQMNSLEPE |
| | | ETAVYYCNARGRATGRDYWGQGTQVTVSS |
| LC (P1AI0188, | 43 | EIVLTQSPATLSLSPGERATLSCRASESVDNYGLSFINWF |
| P1AI5307) | | QQKPGQAPRLLIYGTSNRGSGIPARFSGSGSGTDFTLTISS |
| | | LEPEDFAVYFCQQSNEVPYTFGGGTKVEIKRTVAAPSVFI |
| | | FPPSDRKLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |
| knob chain | 44 | DKTHTCPPCPAPEAAGGPSVFLFPPKPKDTLMISRTPEVT |
| (P1AI0188, | | CVVVDVSHEDPEVKFNWYVDGVEVHNAKTKPREEQYN |
| P1AI5307, | | STYRVVSVLTVLHQDWLNGKEYKCKVSNKALGAPIEKT |
| P1AH6813, | | ISKAKGQPREPQVYTLPPCRDELTKNQVSLWCLVKGFYP |
| P1AI1593) | | SDIAVEWESNGQPENNYKTTPPVLDSDGSFFLYSKLTVD |
| | | KSRWQQGNVFSCSVMHEALHNHYTQKSLSLSPG |
| P1AI0188 hole chain | 45 | QVQLVQSGAEVKKPGASVKVSCKASGYTLTDYNMDW |
| | | VRQAPGQGLEWIGDIYPNTGGTIYNQKFKGRVTMTIDTS |
| | | TSTVYMELSSLRSEDTAVYYCTRFRGIHYAMDYWGQGT |
| | | TVTVSSASTKGPSVFPLAPSSKSTSGGTAALGCLVEDYFP |
| | | EPVTVSWNSGALTSGVHTFPAVLQSSGLYSLSSVVTVPS |
| | | SSLGTQTYICNVNHKPSNTKVDEKVEPKSCDKTHTCPPC |
| | | PAPEAAGGPSVFLFPPKPKDTLMISRTPEVTCVVVDVSH |
| | | EDPEVKFNWYVDGVEVHNAKTKPREEQYNSTYRVVSV |
| | | LTVLHQDWLNGKEYKCKVSNKALGAPIEKTISKAKGQP |
| | | REPQVCTLPPSRDELTKNQVSLSCAVKGFYPSDIAVEWE |
| | | SNGQPENNYKTTPPVLDSDGSFFLVSKLTVDKSRWQQG |
| | | NVFSCSVMHEALHNHYTQKSLSLSPGGGSGGGGSGGGG |
| | | SGGQVQLQEFGGGLVQAGEALRLSCVASKTIFNTMPMG |
| | | WYRQAPGKERELVATITSSGVVNSADSVKGRFTISRDSA |
| | | KRTAYLQMNNLKPEDTAVYYCAAMFKPGIPEYWGRGT |
| | | QVTVSS |
| P1AI5306 knob chain | 46 | EIVLTQSPGTLSLSPGERATLSCRASQSVTSSYLAWYQQ |
| | | KPGQAPRLLINVGSRRATGIPDRFSGSGSGTDFTLTISRLE |
| | | PEDFAVYYCQQGIMLPPTFGQGTKVEIKSSASTKGPSVFP |
| | | LAPSSKSTSGGTAALGCLVKDYFPEPVTVSWNSGALTSG |
| | | VHTFPAVLQSSGLYSLSSVVTVPSSSLGTQTYICNVNHKP |
| | | SNTKVDKKVEPKSCDKTHTCPPCPAPEAAGGPSVFLFPP |
| | | KPKDTLMISRTPEVTCVVVDVSHEDPEVKFNWYVDGVE |
| | | VHNAKTKPREEQYNSTYRVVSVLTVLHQDWLNGKEYK |
| | | CKVSNKALGAPIEKTISKAKGQPREPQVYTLPPCRDELT |
| | | KNQVSLWCLVKGFYPSDIAVEWESNGQPENNYKTTPPV |
| | | LDSDGSFFLYSKLTVDKSRWQQGNVFSCSVMHEALHNH |
| | | YTQKSLSLSPGGGSGGGGSGGGGSGGQVQLQEFGGGLV |
| | | QAGEALRLSCVASKTIFNTMPMGWYRQAPGKERELVAT |
| | | ITSSGVVNSADSVKGRFTISRDSAKRTAYLQMNNLKPED |
| | | TAVYYCAAMFKPGIPEYWGRGTQVTVSS |
| P1AI5307 hole chain | 47 | QVQLVQSGAEVKKPGASVKVSCKASGYTLTDYNMDW |
| | | VRQAPGQGLEWIGDIYPNTGGTIYNQKFKGRVTMTIDTS |
| | | TSTVYMELSSLRSEDTAVYYCTRFRGIHYAMDYWGQGT |
| | | TVTVSSASTKGPSVFPLAPSSKSTSGGTAALGCLVEDYFP |
| | | EPVTVSWNSGALTSGVHTFPAVLQSSGLYSLSSVVTVPS |
| | | SSLGTQTYICNVNHKPSNTKVDEKVEPKSCDKTHTCPPC |
| | | PAPEAAGGPSVFLFPPKPKDTLMISRTPEVTCVVVDVSH |
| | | EDPEVKFNWYVDGVEVHNAKTKPREEQYNSTYRVVSV |
| | | LTVLHQDWLNGKEYKCKVSNKALGAPIEKTISKAKGQP |
| | | REPQVCTLPPSRDELTKNQVSLSCAVKGFYPSDIAVEWE |
| | | SNGQPENNYKTTPPVLDSDGSFFLVSKLTVDKSRWQQG |
| | | NVFSCSVMHEALHNHYTQKSLSLSPGGGSGGGGSGGGG |
| | | SGGQVQLQQSGGGLVQAGGSLRLSCTASGSTFSFSNYH |
| | | MGWYRQAPGKQRERVASISSRDSTYYSDSVKGRFTISRD |
| | | TARNTVYLQMNSLEPEETAVYYCNARGRATGRDYWGQ |
| | | GTQVTVSS |
| P1AI5012 LC 1 | 48 | QVQLQQSGGGLVQAGGSLRLSCTASGSTFSFSNYHMGW |
| | | YRQAPGKQRERVASISSRDSTYYSDSVKGRFTISRDTAR |
| | | NTVYLQMNSLEPEETAVYYCNARGRATGRDYWGQGTQ |
| | | VTVSSGGSGGGGSGGGGSGGGGSGGGGSGGEIVLTQSP |
| | | GTLSLSPGERATLSCRASQSVTSSYLAWYQQKPGQAPRL |
| | | LINVGSRRATGIPDRFSGSGSGTDFTLTISRLEPEDFAVYY |
| | | CQQGIMLPPTFGQGTKVEIKRTVAAPSVFIFPPSDRKLKS |
| | | GTASVVCLLNNFYPREAKVQWKVDNALQSGNSQESVT |
| | | EQDSKDSTYSLSSTLTLSKADYEKHKVYACEVTHQGLSS |
| | | PVTKSFNRGEC |
| P1AI5012 LC 2 | 49 | QVQLQEFGGGLVQAGEALRLSCVASKTIFNTMPMGWY |
| | | RQAPGKERELVATITSSGVVNSADSVKGRFTISRDSAKR |
| | | TAYLQMNNLKPEDTAVYYCAAMFKPGIPEYWGRGTQV |
| | | TVSSGGSGGGGSGGGGSGGGGSGGGGSGGQVQLVQSG |
| | | AEVKKPGASVKVSCKASGYTLTDYNMDWVRQAPGQGL |
| | | EWIGDIYPNTGGTIYNQKFKGRVTMTIDTSTSTVYMELS |
| | | SLRSEDTAVYYCTRFRGIHYAMDYWGQGTTVTVSSASV |
| | | AAPSVFIFPPSDEQLKSGTASVVCLLNNFYPREAKVQWK |
| | | VDNALQSGNSQESVTEQDSKDSTYSLSSTLTLSKADYEK |
| | | HKVYACEVTHQGLSSPVTKSFNRGEC |
| P1AI5012 knob chain | 50 | DKTHTCPPCPAPEAAGGPSVFLFPPKPKDTLMISRTPEVT |
| | | CVVVDVSHEDPEVKFNWYVDGVEVHNAKTKPREEQYN |
| | | STYRVVSVLTVLHQDWLNGKEYKCKVSNKALGAPIEKT |
| | | ISKAKGQPREPQVYTLPPCRDELTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSPGGGSG |
| | | GGGSGGEVQLLESGGGLVQPGGSLRLSCAASGFTFSSYA |
| | | MSWVRQAPGKGLEWVSAIIGSGASTYYADSVKGRFTIS |
| | | RDNSKNTLYLQMNSLRAEDTAVYYCAKGWFGGFNYW |
| | | GQGTLVTVSSASTKGPSVFPLAPSSKSTSGGTAALGCLV |
| | | EDYFPEPVTVSWNSGALTSGVHTFPAVLQSSGLYSLSSV |
| | | VTVPSSSLGTQTYICNVNHKPSNTKVDEKVEPKSC |
| P1AI5012 hole chain | 51 | EIVLTQSPATLSLSPGERATLSCRASESVDNYGLSFINWF |
| | | QQKPGQAPRLLIYGTSNRGSGIPARFSGSGSGTDFTLTISS |
| | | LEPEDFAVYFCQQSNEVPYTFGGGTKVEIKSSASTKGPS |
| | | VFPLAPSSKSTSGGTAALGCLVKDYFPEPVTVSWNSGAL |
| | | TSGVHTFPAVLQSSGLYSLSSVVTVPSSSLGTQTYICNVN |
| | | HKPSNTKVDKKVEPKSCDKTHTCPPCPAPEAAGGPSVFL |
| | | FPPKPKDTLMISRTPEVTCVVVDVSHEDPEVKFNWYVD |
| | | GVEVHNAKTKPREEQYNSTYRVVSVLTVLHQDWLNGK |
| | | EYKCKVSNKALGAPIEKTISKAKGQPREPQVCTLPPSRD |
| | | ELTKNQVSLSCAVKGFYPSDIAVEWESNGQPENNYKTTP |
| | | PVLDSDGSFFLVSKLTVDKSRWQQGNVFSCSVMHEALH |
| | | NHYTQKSLSLSPG |
| IFNγR1 ECD fused to | 52 | EMGTADLGPSSVPTPTNVTIESYNMNPIVYWEYQIMPQV |
| biotinylated Fc knob | | PVFTVEVKNYGVKNSEWIDACINISHHYCNISDHVGDPS |
| (P1AG0843, | | NSLWVRVKARVGQKESAYAKSEEFAVCRDGKIGPPKLD |
| P1AG1987) | | IRKEEKQIMIDIFHPSVFVNGDEQEVDYDPETTCYIRVYN |
| | | VYVRMNGSEIQYKILTQKEDDCDEIQCQLAIPVSSLNSQ |
| | | YCVSAEGVLHVWGVTTEKSKEVCITIVNSSGGGGSGGG |
| | | GDKTHTCPPCPAPELLGGPSVFLFPPKPKDTLMISRTPEV |
| | | TCVVVDVSHEDPEVKFNWYVDGVEVHNAKTKPREEQY |
| | | NSTYRVVSVLTVLHQDWLNGKEYKCKVSNKALPAPIEK |
| | | TISKAKGQPREPQVYTLPPCRDELTKNQVSLWCLVKGFY |
| | | PSDIAVEWESNGQPENNYKTTPPVLDSDGSFFLYSKLTV |
| | | DKSRWQQGNVFSCSVMHEALHNHYTQKSLSLSPGSGGL |
| | | NDIFEAQKIEWHE |
| IFNγR2 C174S ECD | 53 | SQLPAPQHPKIRLYNAEQVLSWEPVALSNSTRPVVYQVQ |
| fused Twin-Strep | | FKYTDSKWFTADIMSIGVNCTQITATECDFTAASPSAGFP |
| tagged Fc hole | | MDFNVTLRLRAELGALHSAWVTMPWFQHYRNVTVGPP |
| (P1AG0843, | | ENIEVTPGEGSLIIRFSSPFDIADTSTAFFSYYVHYWEKGG |
| P1AG6735) | | IQQVKGPFRSNSISLDNLKPSRVYCLQVQAQLLWNKSNI |
| | | FRVGHLSNISCYETMADASTELQQGGGGSGGGGDKTHT |
| | | CPPCPAPELLGGPSVFLFPPKPKDTLMISRTPEVTCVVVD |
| | | VSHEDPEVKFNWYVDGVEVHNAKTKPREEQYNSTYRV |
| | | VSVLTVLHQDWLNGKEYKCKVSNKALPAPIEKTISKAK |
| | | GQPREPQVCTLPPSRDELTKNQVSLSCAVKGFYPSDIAV |
| | | EWESNGQPENNYKTTPPVLDSDGSFFLVSKLTVDKSRW |
| | | QQGNVFSCSVMHEALHNHYTQKSLSLSPGSGGGSAWSH |
| | | PQFEKGGGSGGGSGGSAWSHPQFEK |
| empty Fc hole | 54 | DKTHTCPPCPAPELLGGPSVFLFPPKPKDTLMISRTPEVT |
| (P1AG1987) | | CVVVDVSHEDPEVKFNWYVDGVEVHNAKTKPREEQYN |
| | | STYRVVSVLTVLHQDWLNGKEYKCKVSNKALPAPIEKT |
| | | ISKAKGQPREPQVCTLPPSRDELTKNQVSLSCAVKGFYP |
| | | SDIAVEWESNGQPENNYKTTPPVLDSDGSFFLVSKLTVD |
| | | KSRWQQGNVFSCSVMHEALHNHYTQKSLSLSPG |
| empty biotinylated Fc | 55 | DKTHTCPPCPAPELLGGPSVFLFPPKPKDTLMISRTPEVT |
| knob (P1AG6735, | | CVVVDVSHEDPEVKFNWYVDGVEVHNAKTKPREEQYN |
| P1AE6073, | | STYRVVSVLTVLHQDWLNGKEYKCKVSNKALPAPIEKT |
| P1AF1106) | | ISKAKGQPREPQVYTLPPCRDELTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSPGKSGGL |
| | | NDIFEAQKIEWHE |
| Human Fc AviTag | 56 | GLNDIFEAQKIEWHEDKTHTCPPCPAPELLGGPSVFLFPP |
| (P1AD4290) (knob | | KPKDTLMISRTPEVTCVVVDVSHEDPEVKFNWYVDGVE |
| chain) | | VHNAKTKPREEQYNSTYRVVSVLTVLHQDWLNGKEYK |
| | | CKVSNKALPAPIEKTISKAKGQPREPQVYTLPPCRDELTK |
| | | NQVSLWCLVKGFYPSDIAVEWESNGQPENNYKTTPPVL |
| | | DSDGSFFLYSKLTVDKSRWQQGNVFSCSVMHEALHNH |
| | | YTQKSLSLSPGK |
| Human Fc AviTag | 57 | DKTHTCPPCPAPELLGGPSVFLFPPKPKDTLMISRTPEVT |
| (P1AD4290) (hole | | CVVVDVSHEDPEVKFNWYVDGVEVHNAKTKPREEQYN |
| chain) | | STYRVVSVLTVLHQDWLNGKEYKCKVSNKALPAPIEKT |
| | | ISKAKGQPREPQVCTLPPSRDELTKNQVSLSCAVKGFYP |
| | | SDIAVEWESNGQPENNYKTTPPVLDSDGSFFLVSKLTVD |
| | | KSRWQQGNVFSCSVMHEALHNHYTQKSLSLSPGKSGGL |
| | | NDIFEAQKIEWHE |
| Human Fc AviTag | 58 | DKTHTCPPCPAPELLGGPSVFLFPPKPKDTLMISRTPEVT |
| biotinylated | | CVVVDVSHEDPEVKFNWYVDGVEVHNAKTKPREEQYN |
| (P1AE6073) (hole | | STYRVVSVLTVLHQDWLNGKEYKCKVSNKALPAPIEKT |
| chain) | | ISKAKGQPREPQVCTLPPSRDELTKNQVSLSCAVKGFYP |
| | | SDIAVEWESNGQPENNYKTTPPVLDSDGSFFLVSKLTVD |
| | | KSRWQQGNVFSCSVMHEALHNRFTQKSLSLSPGK |
| IFNgR1_4 heavy | 59 | QVQLQQFGGGLVQAGGSLRLSCTASGSTFSFSNYHMGW |
| chain antibody (knob | | YRQAPGKQRERVASISSRGSTYYSDSVKGRFAISRDTAR |
| chain) | | NTVYLQMNSLEPEETAVYYCNARGRATGRDYWGQGTL |
| | | VTVSSTSGSGGGGSGGGGSGGGGSGGGGSGGGGDKTH |
| | | TCPPCPAPEAAGGPSVFLFPPKPKDTLMISRTPEVTCVVV |
| | | DVSHEDPEVKFNWYVDGVEVHNAKTKPREEQYNSTYR |
| | | VVSVLTVLHQDWLNGKEYKCKVSNKALGAPIEKTISKA |
| | | KGQPREPQVYTLPPCRDELTKNQVSLWCLVKGFYPSDIA |
| | | VEWESNGQPENNYKTTPPVLDSDGSFFLYSKLTVDKSR |
| | | WQQGNVFSCSVMHEALHNHYTQKSLSLSPG |
| IL-2Rb_1 VHH | 60 | QVQLAESGGGLVQPGGSLTLSCVASGSILEMSRMAWYR |
| | | QAPGKMRELVASITSIGSIVYADSVKGRFTLSRDNTKNT |
| | | VYLQMNSLKPEDTAVYRCNALGTWSDETYWGQGTQVT |
| | | VSS |
| IL-2Rb_2 VHH | 61 | QVQLQQFGGGLVQPGGSLTLSCVASESISEMSRMAWYR |
| | | QAPGKQRELVASITMHGGVVYADAVKGRFTISRDNTKN |
| | | TVYLQMNSLKPEDTAVYRCNALGTWSDETYWGQGTLV |
| | | TVSS |
| IL-2Rb_3 VHH | 62 | QVQLQESGGGLVQPGGSLTLSCVASGSILEMSRMAWYR |
| | | QAPGKQRELVASITSIGSKVYADSVKGRFTLSRDNTKNTI |
| | | YLQMNSLKPEDTAVYRCNLLGTWSDETYWGQGTLVTV |
| | | SS |
| IL-2Rb_4 VHH | 63 | QVQLQASGGGLVQSGGSLRLSCVASGSTFSINGMGWYR |
| | | QAPGKERELVATLTAGGNADYAVSVAGRFIISRGDKKN |
| | | TRILQMNDLKPEDTAVYYCMADIYTGDSYSGVDYWGK |
| | | GTLVTVSS |
| IL-2Rb_5 VHH | 64 | QVQLQQSGGGLVQPGGSLTLSCVASGSILEMSRMAWYR |
| | | QAPGKMRELVASITSIGSIVYADSVKGRFTLSRDNTKNT |
| | | VYLQMNSLKPEDTAVYRCNLLGTWSDETYWGQGTQVT |
| | | VSS |
| IL-2Rg_1 VHH | 65 | QVQLQQFGGGSVQAGGSLRLSCSAPGIIFEDIVMGWYRQ |
| | | GPGKQRELVALINSAVTDYADSVKGRFTISRDNAKNLV |
| | | YLQMNSLRSEDTAVYYCTAIDTNWAQYWGQGTQVTVS |
| | | S |
| IL-2Rg_2 VHH | 66 | QVQLQESGGGLVQAGGSLRLSCAASISISKIDLMGWYRQ |
| | | APGRQRELVARVTNGGDSYYSTSVKGRFTISRDNAKNT |
| | | LYLQMNSLKPEDTAVYYCYGVPESLAAFHWGQGTLVT |
| | | VSS |
| IL-2Rg_3 VHH | 67 | QVQLQESGGGLVQPGGSLRLSCAASGSIFSGNAMGWYR |
| | | QAPGKQRELVASITSGGDTHYVDSVKGRFTISRDNAKN |
| | | MVYLQMSSLKPEDTAVYYCNAQERVYSDYAFASWGPG |
| | | TQVTVSS |
| IL-2Rg_4 VHH | 68 | QVQLQESGGGLVQPGGSLRLSCAASGFTFSTYVMNWVR |
| | | QAPGKGLEWVSGIDSDGDSTTYTDSVKGRFTISRDNAK |
| | | NTLYLQMNSLKPEDTAVYYCSMFRMATTGSQGTQVTV |
| | | SS |
| IL-2Rg_5 VHH | 69 | QVQLQESGGGLVQAGGSLRLSCAASGITSSIYAMGWYR |
| | | QAPGNEREPVALITSGDNTNYPDSVKGRFTISRDTAKNT |
| | | VYLQMSSLKPEDTAVYYCYADVVIGTTYYTSWGQGTL |
| | | VTVSS |
| IL-2Rb_1 heavy chain | 70 | QVQLAESGGGLVQPGGSLTLSCVASGSILEMSRMAWYR |
| antibody (knob chain) | | QAPGKMRELVASITSIGSIVYADSVKGRFTLSRDNTKNT |
| | | VYLQMNSLKPEDTAVYRCNALGTWSDETYWGQGTQVT |
| | | VSSTSGSGGGGSGGGGSGGGGSGGGGSGGGGDKTHTCP |
| | | PCPAPEAAGGPSVFLFPPKPKDTLMISRTPEVTCVVVDVS |
| | | HEDPEVKFNWYVDGVEVHNAKTKPREEQYNSTYRVVS |
| | | VLTVLHQDWLNGKEYKCKVSNKALGAPIEKTISKAKGQ |
| | | PREPQVYTLPPCRDELTKNQVSLWCLVKGFYPSDIAVE |
| | | WESNGQPENNYKTTPPVLDSDGSFFLYSKLTVDKSRWQ |
| | | QGNVFSCSVMHEALHNHYTQKSLSLSPG |
| IL-2Rb_2 heavy chain | 71 | QVQLQQFGGGLVQPGGSLTLSCVASESISEMSRMAWYR |
| antibody (knob chain) | | QAPGKQRELVASITMHGGVVYADAVKGRFTISRDNTKN |
| | | TVYLQMNSLKPEDTAVYRCNALGTWSDETYWGQGTLV |
| | | TVSSTSGSGGGGSGGGGSGGGGSGGGGSGGGGDKTHTC |
| | | PPCPAPEAAGGPSVFLFPPKPKDTLMISRTPEVTCVVVDV |
| | | SHEDPEVKFNWYVDGVEVHNAKTKPREEQYNSTYRVV |
| | | SVLTVLHQDWLNGKEYKCKVSNKALGAPIEKTISKAKG |
| | | QPREPQVYTLPPCRDELTKNQVSLWCLVKGFYPSDIAVE |
| | | WESNGQPENNYKTTPPVLDSDGSFFLYSKLTVDKSRWQ |
| | | QGNVFSCSVMHEALHNHYTQKSLSLSPG |
| IL-2Rb_3 heavy chain | 72 | QVQLQESGGGLVQPGGSLTLSCVASGSILEMSRMAWYR |
| antibody(knob chain) | | QAPGKQRELVASITSIGSKVYADSVKGRFTLSRDNTKNTI |
| | | YLQMNSLKPEDTAVYRCNLLGTWSDETYWGQGTLVTV |
| | | SSTSGSGGGGSGGGGSGGGGSGGGGSGGGGDKTHTCPP |
| | | CPAPEAAGGPSVFLFPPKPKDTLMISRTPEVTCVVVDVS |
| | | HEDPEVKFNWYVDGVEVHNAKTKPREEQYNSTYRVVS |
| | | VLTVLHQDWLNGKEYKCKVSNKALGAPIEKTISKAKGQ |
| | | PREPQVYTLPPCRDELTKNQVSLWCLVKGFYPSDIAVE |
| | | WESNGQPENNYKTTPPVLDSDGSFFLYSKLTVDKSRWQ |
| | | QGNVFSCSVMHEALHNHYTQKSLSLSPG |
| IL-2Rb_5 heavy chain | 73 | QVQLQQSGGGLVQPGGSLTLSCVASGSILEMSRMAWYR |
| antibody (knob chain) | | QAPGKMRELVASITSIGSIVYADSVKGRFTLSRDNTKNT |
| | | VYLQMNSLKPEDTAVYRCNLLGTWSDETYWGQGTQVT |
| | | VSSTSGSGGGGSGGGGSGGGGSGGGGSGGGGDKTHTCP |
| | | PCPAPEAAGGPSVFLFPPKPKDTLMISRTPEVTCVVVDVS |
| | | HEDPEVKFNWYVDGVEVHNAKTKPREEQYNSTYRVVS |
| | | VLTVLHQDWLNGKEYKCKVSNKALGAPIEKTISKAKGQ |
| | | PREPQVYTLPPCRDELTKNQVSLWCLVKGFYPSDIAVE |
| | | WESNGQPENNYKTTPPVLDSDGSFFLYSKLTVDKSRWQ |
| | | QGNVFSCSVMHEALHNHYTQKSLSLSPG |
| IL-2Rg_2 heavy chain | 74 | QVQLQESGGGLVQAGGSLRLSCAASISISKIDLMGWYRQ |
| antibody (hole chain) | | APGRQRELVARVTNGGDSYYSTSVKGRFTISRDNAKNT |
| | | LYLQMNSLKPEDTAVYYCYGVPESLAAFHWGQGTLVT |
| | | VSSTSGSGGGGSGGGGSGGGGSGGGGSGGGGDKTHTCP |
| | | PCPAPEAAGGPSVFLFPPKPKDTLMISRTPEVTCVVVDVS |
| | | HEDPEVKFNWYVDGVEVHNAKTKPREEQYNSTYRVVS |
| | | VLTVLHQDWLNGKEYKCKVSNKALGAPIEKTISKAKGQ |
| | | PREPQVCTLPPSRDELTKNQVSLSCAVKGFYPSDIAVEW |
| | | ESNGQPENNYKTTPPVLDSDGSFFLVSKLTVDKSRWQQ |
| | | GNVFSCSVMHEALHNHYTQKSLSLSPG |
| IL-2Rg_3 heavy chain | 75 | QVQLQESGGGLVQPGGSLRLSCAASGSIFSGNAMGWYR |
| antibody (hole chain) | | QAPGKQRELVASITSGGDTHYVDSVKGRFTISRDNAKN |
| | | MVYLQMSSLKPEDTAVYYCNAQERVYSDYAFASWGPG |
| | | TQVTVSSTSGSGGGGSGGGGSGGGGSGGGGSGGGGDK |
| | | THTCPPCPAPEAAGGPSVFLFPPKPKDTLMISRTPEVTCV |
| | | VVDVSHEDPEVKFNWYVDGVEVHNAKTKPREEQYNST |
| | | YRVVSVLTVLHQDWLNGKEYKCKVSNKALGAPIEKTIS |
| | | KAKGQPREPQVCTLPPSRDELTKNQVSLSCAVKGFYPSD |
| | | IAVEWESNGQPENNYKTTPPVLDSDGSFFLVSKLTVDKS |
| | | RWQQGNVFSCSVMHEALHNHYTQKSLSLSPG |
| IL-2Rg_4 heavy chain | 76 | QVQLQESGGGLVQPGGSLRLSCAASGFTFSTYVMNWVR |
| antibody(hole chain) | | QAPGKGLEWVSGIDSDGDSTTYTDSVKGRFTISRDNAK |
| | | NTLYLQMNSLKPEDTAVYYCSMFRMATTGSQGTQVTV |
| | | SSTSGSGGGGSGGGGSGGGGSGGGGSGGGGDKTHTCPP |
| | | CPAPEAAGGPSVFLFPPKPKDTLMISRTPEVTCVVVDVS |
| | | HEDPEVKFNWYVDGVEVHNAKTKPREEQYNSTYRVVS |
| | | VLTVLHQDWLNGKEYKCKVSNKALGAPIEKTISKAKGQ |
| | | PREPQVCTLPPSRDELTKNQVSLSCAVKGFYPSDIAVEW |
| | | ESNGQPENNYKTTPPVLDSDGSFFLVSKLTVDKSRWQQ |
| | | GNVFSCSVMHEALHNHYTQKSLSLSPG |
| IL-2Rg_5 heavy chain | 77 | QVQLQESGGGLVQAGGSLRLSCAASGITSSIYAMGWYR |
| antibody (hole chain) | | QAPGNEREPVALITSGDNTNYPDSVKGRFTISRDTAKNT |
| | | VYLQMSSLKPEDTAVYYCYADVVIGTTYYTSWGQGTL |
| | | VTVSSTSGSGGGGSGGGGSGGGGSGGGGSGGGGDKTH |
| | | TCPPCPAPEAAGGPSVFLFPPKPKDTLMISRTPEVTCVVV |
| | | DVSHEDPEVKFNWYVDGVEVHNAKTKPREEQYNSTYR |
| | | VVSVLTVLHQDWLNGKEYKCKVSNKALGAPIEKTISKA |
| | | KGQPREPQVCTLPPSRDELTKNQVSLSCAVKGFYPSDIA |
| | | VEWESNGQPENNYKTTPPVLDSDGSFFLVSKLTVDKSR |
| | | WQQGNVFSCSVMHEALHNHYTQKSLSLSPG |
| IL-2Rβ_4 heavy chain | 78 | QVQLQASGGGLVQSGGSLRLSCVASGSTFSINGMGWYR |
| antibody (knob chain) | | QAPGKERELVATLTAGGNADYAVSVAGRFIISRGDKKN |
| | | TRILQMNDLKPEDTAVYYCMADIYTGDSYSGVDYWGK |
| | | GTLVTVSSTSGSGGGGSGGGGSGGGGGDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLT |
| | | VLHQDWLNGKEYKCKVSNKALGAPIEKTISKAKGQPRE |
| | | PQVYTLPPCRDELTKNQVSLWCLVKGFYPSDIAVEWES |
| | | NGQPENNYKTTPPVLDSDGSFFLYSKLTVDKSRWQQGN |
| | | VFSCSVMHEALHNHYTQKSLSLSPG |
| IL-2Rγ_1 heavy chain | 79 | QVQLQQFGGGSVQAGGSLRLSCSAPGIIFEDIVMGWYRQ |
| abtibody (hole chain) | | GPGKQRELVALINSAVTDYADSVKGRFTISRDNAKNLV |
| | | YLQMNSLRSEDTAVYYCTAIDTNWAQYWGQGTQVTVS |
| | | STSGSGGGGSGGGGSGGGGSGGGGSGGGGDKTHTCPPC |
| | | PAPEAAGGPSVFLFPPKPKDTLMISRTPEVTCVVVDVSH |
| | | EDPEVKFNWYVDGVEVHNAKTKPREEQYNSTYRVVSV |
| | | LTVLHQDWLNGKEYKCKVSNKALGAPIEKTISKAKGQP |
| | | REPQVCTLPPSRDELTKNQVSLSCAVKGFYPSDIAVEWE |
| | | SNGQPENNYKTTPPVLDSDGSFFLVSKLTVDKSRWQQG |
| | | NVFSCSVMHEALHNHYTQKSLSLSPG |
| PD-1 binder 1 0376 | 80 | EVQLLESGGGLVQPGGSLRLSCAASGFSFSSYTMSWVR |
| VH | | QAPGKGLEWVATISGGGRDIYYPDSVKGRFTISRDNSKN |
| | | TLYLQMNSLRAEDTAVYYCVLLTGRVYFALDSWGQGT |
| | | LVTVSS |
| PD-1 binder 1 0376 | 81 | DIVMTQSPDSLAVSLGERATINCKASESVDTSDNSFIHW |
| VL | | YQQKPGQSPKLLIYRSSTLESGVPDRFSGSGSGTDFTLTIS |
| | | SLQAEDVAVYYCQQNYDVPWTFGQGTKVEIK |
| PD-1 binder 2 1040 | 82 | EVQLLESGGGLVQPGGSLRLSCAASGFTFSSYAMSWVR |
| VH | | QAPGKGLEWVSAITGSGGSTYYADSVKGRFTISRDNSRN |
| | | TLYLQMNSLRAEDTAVYYCAKGEGYAGSSYFRASDIW |
| | | GQGTMVTVSS |
| PD-1 binder 2 1040 | 83 | DIQMTQSPSSLSASVGDRVTITCRASQSISSYLNWYQQKP |
| VL | | GKAPKLLIYTASSLQSGVPSRFSGSGSGTDFTLTISSLQPE |
| | | DFATYYCQQSYSTPLTFGGGTKVEIK |
| human IL-2Rβ Fc | 84 | AVNGTSQFTCFYNSRANISCVWSQDGALQDTSCQVHA |
| biotinylated | | WPDRRRWNQTCELLPVSQASWACNLILGAPDSQKLTTV |
| (P1AE2657, | | DIVTLRVLCREGVRWRVMAIQDFKPFENLRLMAPISLQV |
| P1AF1104) (knob | | VHVETHRCNISWEISQASHYFERHLEFEARTLSPGHTWE |
| chain) | | EAPLLTLKQKQEWICLETLTPDTQYEFQVRVKPLQGEFT |
| | | TWSPWSQPLAFRTKPAALGKDTGAQDKTHTCPPCPAPE |
| | | LLGGPSVFLFPPKPKDTLMISRTPEVTCVVVDVSHEDPEV |
| | | KFNWYVDGVEVHNAKTKPREEQYNSTYRVVSVLTVLH |
| | | QDWLNGKEYKCKVSNKALPAPIEKTISKAKGQPREPQV |
| | | YTLPPCRDELTKNQVSLWCLVKGFYPSDIAVEWESNGQ |
| | | PENNYKTTPPVLDSDGSFFLYSKLTVDKSRWQQGNVFS |
| | | CSVMHEALHNHYTQKSLSLSPGKSGGLNDIFEAQKIEW |
| | | HE |
| human IL-2Rγ Fc | 85 | LNTTILTPNGNEDTTADFFLTTMPTDSLSVSTLPLPEVQC |
| (P1AE2657, | | FVFNVEYMNCTWNSSSEPQPTNLTLHYWYKNSDNDKV |
| P1AF1106) (hole | | QKCSHYLFSEEITSGCQLQKKEIHLYQTFVVQLQDPREPR |
| chain) | | RQATQMLKLQNLVIPWAPENLTLHKLSESQLELNWNNR |
| | | FLNHCLEHLVQYRTDWDHSWTEQSVDYRHKFSLPSVD |
| | | GQKRYTFRVRSRFNPLCGSAQHWSEWSHPIHWGSNTSK |
| | | ENPFLFALEAGAQDKTHTCPPCPAPELLGGPSVFLFPPKP |
| | | KDTLMISRTPEVTCVVVDVSHEDPEVKFNWYVDGVEVH |
| | | NAKTKPREEQYNSTYRVVSVLTVLHQDWLNGKEYKCK |
| | | VSNKALPAPIEKTISKAKGQPREPQVCTLPPSRDELTKNQ |
| | | VSLSCAVKGFYPSDIAVEWESNGQPENNYKTTPPVLDSD |
| | | GSFFLVSKLTVDKSRWQQGNVFSCSVMHEALHNHYTQ |
| | | KSLSLSPGK |
| Monovalent human | 86 | DKTHTCPPCPAPELLGGPSVFLFPPKPKDTLMISRTPEVT |
| IL-2Rβ Fc | | CVVVDVSHEDPEVKFNWYVDGVEVHNAKTKPREEQYN |
| biotinylated | | STYRVVSVLTVLHQDWLNGKEYKCKVSNKALPAPIEKT |
| (P1AF1104) (hole | | ISKAKGQPREPQVCTLPPSRDELTKNQVSLSCAVKGFYP |
| chain) | | SDIAVEWESNGQPENNYKTTPPVLDSDGSFFLVSKLTVD |
| | | KSRWQQGNVFSCSVMHEALHNHYTQKSLSLSPGK |
| Anti-PD1 LC | 87 | DIVMTQSPDSLAVSLGERATINCKASESVDTSDNSFIHW |
| (P1AE4422, | | YQQKPGQSPKLLIYRSSTLESGVPDRFSGSGSGTDFTLTIS |
| P1AH6813) | | SLQAEDVAVYYCQQNYDVPWTFGQGTKVEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| Anti-PD1-IL2v | 88 | EVQLLESGGGLVQPGGSLRLSCAASGFSFSSYTMSWVR |
| (P1AE4422) knob | | QAPGKGLEWVATISGGGRDIYYPDSVKGRFTISRDNSKN |
| chain | | TLYLQMNSLRAEDTAVYYCVLLTGRVYFALDSWGQGT |
| | | LVTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKKVEPKSCDKTHTCPPC |
| | | PAPEAAGGPSVFLFPPKPKDTLMISRTPEVTCVVVDVSH |
| | | EDPEVKFNWYVDGVEVHNAKTKPREEQYNSTYRVVSV |
| | | LTVLHQDWLNGKEYKCKVSNKALGAPIEKTISKAKGQP |
| | | REPQVYTLPPCRDELTKNQVSLWCLVKGFYPSDIAVEW |
| | | ESNGQPENNYKTTPPVLDSDGSFFLYSKLTVDKSRWQQ |
| | | GNVFSCSVMHEALHNHYTQKSLSLSPGGGGGSGGGGSG |
| | | GGGSAPASSSTKKTQLQLEHLLLDLQMILNGINNYKNPK |
| | | LTRMLTAKFAMPKKATELKHLQCLEEELKPLEEVLNGA |
| | | QSKNFHLRPRDLISNINVIVLELKGSETTFMCEYADETAT |
| | | IVEFLNRWITFAQSIISTLT |
| Anti-PD1-IL2v | 89 | EVQLLESGGGLVQPGGSLRLSCAASGFSFSSYTMSWVR |
| (P1AE4422) hole | | QAPGKGLEWVATISGGGRDIYYPDSVKGRFTISRDNSKN |
| chain | | TLYLQMNSLRAEDTAVYYCVLLTGRVYFALDSWGQGT |
| | | LVTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKKVEPKSCDKTHTCPPC |
| | | PAPEAAGGPSVFLFPPKPKDTLMISRTPEVTCVVVDVSH |
| | | EDPEVKFNWYVDGVEVHNAKTKPREEQYNSTYRVVSV |
| | | LTVLHQDWLNGKEYKCKVSNKALGAPIEKTISKAKGQP |
| | | REPQVCTLPPSRDELTKNQVSLSCAVKGFYPSDIAVEWE |
| | | SNGQPENNYKTTPPVLDSDGSFFLVSKLTVDKSRWQQG |
| | | NVFSCSVMHEALHNHYTQKSLSLSPGK |
| P1AH6813 hole chain | 90 | QVQLQQSGGGLVQPGGSLTLSCVASGSILEMSRMAWYR |
| | | QAPGKMRELVASITSIGSIVYADSVKGRFTLSRDNTKNT |
| | | VYLQMNSLKPEDTAVYRCNLLGTWSDETYWGQGTQVT |
| | | VSSTSGSGGGGSGGGGSGGGGSGGGGSGGGGEVQLLES |
| | | GGGLVQPGGSLRLSCAASGFSFSSYTMSWVRQAPGKGL |
| | | EWVATISGGGRDIYYPDSVKGRFTISRDNSKNTLYLQMN |
| | | SLRAEDTAVYYCVLLTGRVYFALDSWGQGTLVTVSSAS |
| | | TKGPSVFPLAPSSKSTSGGTAALGCLVKDYFPEPVTVSW |
| | | NSGALTSGVHTFPAVLQSSGLYSLSSVVTVPSSSLGTQTY |
| | | ICNVNHKPSNTKVDKKVEPKSCDKTHTCPPCPAPEAAG |
| | | GPSVFLFPPKPKDTLMISRTPEVTCVVVDVSHEDPEVKF |
| | | NWYVDGVEVHNAKTKPREEQYNSTYRVVSVLTVLHQD |
| | | WLNGKEYKCKVSNKALGAPIEKTISKAKGQPREPQVCT |
| | | LPPSRDELTKNQVSLSCAVKGFYPSDIAVEWESNGQPEN |
| | | NYKTTPPVLDSDGSFFLVSKLTVDKSRWQQGNVFSCSV |
| | | MHEALHNHYTQKSLSLSPG |
| P1AH6850 LC | 91 | QVQLQESGGGLVQAGGSLRLSCAASISISKIDLMGWYRQ |
| | | APGRQRELVARVTNGGDSYYSTSVKGRFTISRDNAKNT |
| | | LYLQMNSLKPEDTAVYYCYGVPESLAAFHWGQGTLVT |
| | | VSSTSGSGGGGSGGGGSGGGGSGGGGSGGGGDIQMTQS |
| | | PSSLSASVGDRVTITCRASQSISSYLNWYQQKPGKAPKL |
| | | LIYTASSLQSGVPSRFSGSGSGTDFTLTISSLQPEDFATYY |
| | | CQQSYSTPLTFGGGTKVEIKRTVAAPSVFIFPPSDEQLKS |
| | | GTASVVCLLNNFYPREAKVQWKVDNALQSGNSQESVT |
| | | EQDSKDSTYSLSSTLTLSKADYEKHKVYACEVTHQGLSS |
| | | PVTKSFNRGEC |
| P1AH6850, | 92 | EVQLLESGGGLVQPGGSLRLSCAASGFTFSSYAMSWVR |
| P1AK2798 knob | | QAPGKGLEWVSAITGSGGSTYYADSVKGRFTISRDNSRN |
| chain | | TLYLQMNSLRAEDTAVYYCAKGEGYAGSSYFRASDIW |
| | | GQGTMVTVSSASTKGPSVFPLAPSSKSTSGGTAALGCLV |
| | | KDYFPEPVTVSWNSGALTSGVHTFPAVLQSSGLYSLSSV |
| | | VTVPSSSLGTQTYICNVNHKPSNTKVDKKVEPKSCDKTH |
| | | TCPPCPAPEAAGGPSVFLFPPKPKDTLMISRTPEVTCVVV |
| | | DVSHEDPEVKFNWYVDGVEVHNAKTKPREEQYNSTYR |
| | | VVSVLTVLHQDWLNGKEYKCKVSNKALGAPIEKTISKA |
| | | KGQPREPQVYTLPPCRDELTKNQVSLWCLVKGFYPSDIA |
| | | VEWESNGQPENNYKTTPPVLDSDGSFFLYSKLTVDKSR |
| | | WQQGNVFSCSVMHEALHNHYTQKSLSLSPG |
| P1AH6814, | 93 | DIQMTQSPSSLSASVGDRVTITCRASQSISSYLNWYQQKP |
| P1AK2599, | | GKAPKLLIYTASSLQSGVPSRFSGSGSGTDFTLTISSLQPE |
| P1AK2806 LC | | DFATYYCQQSYSTPLTFGGGTKVEIKRTVAAPSVFIFPPS |
| | | DEQLKSGTASVVCLLNNFYPREAKVQWKVDNALQSGN |
| | | SQESVTEQDSKDSTYSLSSTLTLSKADYEKHKVYACEVT |
| | | HQGLSSPVTKSFNRGEC |
| P1AH6814 knob | 94 | QVQLQESGGGLVQAGGSLRLSCAASISISKIDLMGWYRQ |
| chain | | APGRQRELVARVINGGDSYYSTSVKGRFTISRDNAKNT |
| | | LYLQMNSLKPEDTAVYYCYGVPESLAAFHWGQGTLVT |
| | | VSSTSGSGGGGSGGGGSGGGGSGGGGSGGGGEVQLLES |
| | | GGGLVQPGGSLRLSCAASGFTFSSYAMSWVRQAPGKGL |
| | | EWVSAITGSGGSTYYADSVKGRFTISRDNSRNTLYLQM |
| | | NSLRAEDTAVYYCAKGEGYAGSSYFRASDIWGQGTMV |
| | | TVSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCPA |
| | | PEAAGGPSVFLFPPKPKDTLMISRTPEVTCVVVDVSHED |
| | | PEVKFNWYVDGVEVHNAKTKPREEQYNSTYRVVSVLT |
| | | VLHQDWLNGKEYKCKVSNKALGAPIEKTISKAKGQPRE |
| | | PQVYTLPPCRDELTKNQVSLWCLVKGFYPSDIAVEWES |
| | | NGQPENNYKTTPPVLDSDGSFFLYSKLTVDKSRWQQGN |
| | | VFSCSVMHEALHNHYTQKSLSLSPG |
| P1AI1593 LC | 95 | QVQLQQSGGGLVQPGGSLTLSCVASGSILEMSRMAWYR |
| | | QAPGKMRELVASITSIGSIVYADSVKGRFTLSRDNTKNT |
| | | VYLQMNSLKPEDTAVYRCNLLGTWSDETYWGQGTQVT |
| | | VSSTSGSGGGGSGGGGSGGGGSGGGGSGGGGDIVMTQS |
| | | PDSLAVSLGERATINCKASESVDTSDNSFIHWYQQKPGQ |
| | | SPKLLIYRSSTLESGVPDRFSGSGSGTDFTLTISSLQAEDV |
| | | AVYYCQQNYDVPWTFGQGTKVEIKRTVAAPSVFIFPPSD |
| | | EQLKSGTASVVCLLNNFYPREAKVQWKVDNALQSGNS |
| | | QESVTEQDSKDSTYSLSSTLTLSKADYEKHKVYACEVTH |
| | | QGLSSPVTKSFNRGEC |
| P1AI1593 hole chain | 96 | EVQLLESGGGLVQPGGSLRLSCAASGFSFSSYTMSWVR |
| | | QAPGKGLEWVATISGGGRDIYYPDSVKGRFTISRDNSKN |
| | | TLYLQMNSLRAEDTAVYYCVLLTGRVYFALDSWGQGT |
| | | LVTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKKVEPKSCDKTHTCPPC |
| | | PAPEAAGGPSVFLFPPKPKDTLMISRTPEVTCVVVDVSH |
| | | EDPEVKFNWYVDGVEVHNAKTKPREEQYNSTYRVVSV |
| | | LTVLHQDWLNGKEYKCKVSNKALGAPIEKTISKAKGQP |
| | | REPQVCTLPPSRDELTKNQVSLSCAVKGFYPSDIAVEWE |
| | | SNGQPENNYKTTPPVLDSDGSFFLVSKLTVDKSRWQQG |
| | | NVFSCSVMHEALHNHYTQKSLSLSPG |
| P1AJ0690 LC1 | 97 | QVQLQEFGGGLVQAGEALRLSCVASKTIFNTMPMGWY |
| | | RQAPGKERELVATITSSGVVNSADSVKGRFTISRDSAKR |
| | | TAYLQMNNLKPEDTAVYYCAAMFKPGIPEYWGRGTQV |
| | | TVSSGGSGGQVQLVQSGAEVKKPGASVKVSCKASGYTL |
| | | TDYNMDWVRQAPGQGLEWIGDIYPNTGGTIYNQKFKG |
| | | RVTMTIDTSTSTVYMELSSLRSEDTAVYYCTRFRGIHYA |
| | | MDYWGQGTTVTVSSASTKGPSVFPLAPSSKSTSGGTAA |
| | | LGCLVEDYFPEPVTVSWNSGALTSGVHTFPAVLQSSGLY |
| | | SLSSVVTVPSSSLGTQTYICNVNHKPSNTKVDEKVEPKS |
| | | CD |
| P1AJ0690 LC2 | 98 | QVQLQQSGGGLVQAGGSLRLSCTASGSTFSFSNYHMGW |
| | | YRQAPGKQRERVASISSRDSTYYSDSVKGRFTISRDTAR |
| | | NTVYLQMNSLEPEETAVYYCNARGRATGRDYWGQGTQ |
| | | VTVSSGGSGGEIVLTQSPGTLSLSPGERATLSCRASQSVT |
| | | SSYLAWYQQKPGQAPRLLINVGSRRATGIPDRFSGSGSG |
| | | TDFTLTISRLEPEDFAVYYCQQGIMLPPTFGQGTKVEIKS |
| | | SASTKGPSVFPLAPSSKSTSGGTAALGCLVKDYFPEPVTV |
| | | SWNSGALTSGVHTFPAVLQSSGLYSLSSVVTVPSSSLGT |
| | | QTYICNVNHKPSNTKVDKKVEPKSCD |
| knob chain | 99 | DKTHTCPPCPAPEAAGGPSVFLFPPKPKDTLMISRTPEVT |
| (P1AJ0690, | | CVVVDVSHEDPEVKFNWYVDGVEVHNAKTKPREEQYN |
| P1AJ0685, | | STYRVVSVLTVLHQDWLNGKEYKCKVSNKALGAPIEKT |
| P1AJ0735) | | ISKAKGQPREPQVYTLPPCRDELTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSPGGGSG |
| | | GGGSGGEIVLTQSPATLSLSPGERATLSCRASESVDNYGL |
| | | SFINWFQQKPGQAPRLLIYGTSNRGSGIPARFSGSGSGTD |
| | | FTLTISSLEPEDFAVYFCQQSNEVPYTFGGGTKVEIKRTV |
| | | AAPSVFIFPPSDRKLKSGTASVVCLLNNFYPREAKVQWK |
| | | VDNALQSGNSQESVTEQDSKDSTYSLSSTLTLSKADYEK |
| | | HKVYACEVTHQGLSSPVTKSFNRGEC |
| hole chain | 100 | EVQLLESGGGLVQPGGSLRLSCAASGFTFSSYAMSWVR |
| (P1AJ0690, | | QAPGKGLEWVSAIIGSGASTYYADSVKGRFTISRDNSKN |
| P1AJ0685, | | TLYLQMNSLRAEDTAVYYCAKGWFGGFNYWGQGTLV |
| P1AJ0735) | | TVSSASVAAPSVFIFPPSDEQLKSGTASVVCLLNNFYPRE |
| | | AKVQWKVDNALQSGNSQESVTEQDSKDSTYSLSSTLTL |
| | | SKADYEKHKVYACEVTHQGLSSPVTKSFNRGECDKTHT |
| | | CPPCPAPEAAGGPSVFLFPPKPKDTLMASRTPEVTCVVV |
| | | DVSHEDPEVKFNWYVDGVEVHNAKTKPREEQYNSTYR |
| | | VVSVLTVLAQDWLNGKEYKCKVSNKALGAPIEKTISKA |
| | | KGQPREPQVCTLPPSRDELTKNQVSLSCAVKGFYPSDIA |
| | | VEWESNGQPENNYKTTPPVLDSDGSFFLVSKLTVDKSR |
| | | WQQGNVFSCSVMHEALHNAYTQKSLSLSPG |
| P1AJ0672 LC1 | 101 | QVQLQEFGGGLVQAGEALRLSCVASKTIFNTMPMGWY |
| | | RQAPGKERELVATITSSGVVNSADSVKGRFTISRDSAKR |
| | | TAYLQMNNLKPEDTAVYYCAAMFKPGIPEYWGRGTQV |
| | | TVSSGGSGGEIVLTQSPATLSLSPGERATLSCRASESVDN |
| | | YGLSFINWFQQKPGQAPRLLIYGTSNRGSGIPARFSGSGS |
| | | GTDFTLTISSLEPEDFAVYFCQQSNEVPYTFGGGTKVEIK |
| | | RTVAAPSVFIFPPSDRKLKSGTASVVCLLNNFYPREAKV |
| | | QWKVDNALQSGNSQESVTEQDSKDSTYSLSSTLTLSKA |
| | | DYEKHKVYACEVTHQGLSSPVTKSFNRGEC |
| P1AJ0672 LC2 | 102 | QVQLQQSGGGLVQAGGSLRLSCTASGSTFSFSNYHMGW |
| | | YRQAPGKQRERVASISSRDSTYYSDSVKGRFTISRDTAR |
| | | NTVYLQMNSLEPEETAVYYCNARGRATGRDYWGQGTQ |
| | | VTVSSGGSGGEVQLLESGGGLVQPGGSLRLSCAASGFTF |
| | | SSYAMSWVRQAPGKGLEWVSAIIGSGASTYYADSVKGR |
| | | FTISRDNSKNTLYLQMNSLRAEDTAVYYCAKGWFGGFN |
| | | YWGQGTLVTVSSASVAAPSVFIFPPSDEQLKSGTASVVC |
| | | LLNNFYPREAKVQWKVDNALQSGNSQESVTEQDSKDST |
| | | YSLSSTLTLSKADYEKHKVYACEVTHQGLSSPVTKSFNR |
| | | GEC |
| P1AJ0672 knob chain | 103 | DKTHTCPPCPAPEAAGGPSVFLFPPKPKDTLMISRTPEVT |
| | | CVVVDVSHEDPEVKFNWYVDGVEVHNAKTKPREEQYN |
| | | STYRVVSVLTVLHQDWLNGKEYKCKVSNKALGAPIEKT |
| | | ISKAKGQPREPQVYTLPPCRDELTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSPGGGSG |
| | | GGGSGGQVQLVQSGAEVKKPGASVKVSCKASGYTLTD |
| | | YNMDWVRQAPGQGLEWIGDIYPNTGGTIYNQKFKGRV |
| | | TMTIDTSTSTVYMELSSLRSEDTAVYYCTRFRGIHYAMD |
| | | YWGQGTTVTVSSASTKGPSVFPLAPSSKSTSGGTAALGC |
| | | LVEDYFPEPVTVSWNSGALTSGVHTFPAVLQSSGLYSLS |
| | | SVVTVPSSSLGTQTYICNVNHKPSNTKVDEKVEPKSCD |
| P1AJ0672 knob chain | 104 | EIVLTQSPGTLSLSPGERATLSCRASQSVTSSYLAWYQQ |
| | | KPGQAPRLLINVGSRRATGIPDRFSGSGSGTDFTLTISRLE |
| | | PEDFAVYYCQQGIMLPPTFGQGTKVEIKSSASTKGPSVFP |
| | | LAPSSKSTSGGTAALGCLVKDYFPEPVTVSWNSGALTSG |
| | | VHTFPAVLQSSGLYSLSSVVTVPSSSLGTQTYICNVNHKP |
| | | SNTKVDKKVEPKSCDKTHTCPPCPAPEAAGGPSVFLFPP |
| | | KPKDTLMASRTPEVTCVVVDVSHEDPEVKFNWYVDGV |
| | | EVHNAKTKPREEQYNSTYRVVSVLTVLAQDWLNGKEY |
| | | KCKVSNKALGAPIEKTISKAKGQPREPQVCTLPPSRDELT |
| | | KNQVSLSCAVKGFYPSDIAVEWESNGQPENNYKTTPPV |
| | | LDSDGSFFLVSKLTVDKSRWQQGNVFSCSVMHEALHNA |
| | | YTQKSLSLSPG |
| P1AJ0700 LC1 | 105 | QVQLQEFGGGLVQAGEALRLSCVASKTIFNTMPMGWY |
| | | RQAPGKERELVATITSSGVVNSADSVKGRFTISRDSAKR |
| | | TAYLQMNNLKPEDTAVYYCAAMFKPGIPEYWGRGTQV |
| | | TVSSGGSGGEIVLTQSPGTLSLSPGERATLSCRASQSVTS |
| | | SYLAWYQQKPGQAPRLLINVGSRRATGIPDRFSGSGSGT |
| | | DFTLTISRLEPEDFAVYYCQQGIMLPPTFGQGTKVEIKRT |
| | | VAAPSVFIFPPSDRKLKSGTASVVCLLNNFYPREAKVQW |
| | | KVDNALQSGNSQESVTEQDSKDSTYSLSSTLTLSKADYE |
| | | KHKVYACEVTHQGLSSPVTKSFNRGEC |
| P1AJ0700 LC2 | 106 | QVQLQQSGGGLVQAGGSLRLSCTASGSTFSFSNYHMGW |
| | | YRQAPGKQRERVASISSRDSTYYSDSVKGRFTISRDTAR |
| | | NTVYLQMNSLEPEETAVYYCNARGRATGRDYWGQGTQ |
| | | VTVSSGGSGGEIVLTQSPATLSLSPGERATLSCRASESVD |
| | | NYGLSFINWFQQKPGQAPRLLIYGTSNRGSGIPARFSGSG |
| | | SGTDFTLTISSLEPEDFAVYFCQQSNEVPYTFGGGTKVEI |
| | | KSSASTKGPSVFPLAPSSKSTSGGTAALGCLVKDYFPEPV |
| | | TVSWNSGALTSGVHTFPAVLQSSGLYSLSSVVTVPSSSL |
| | | GTQTYICNVNHKPSNTKVDKKVEPKSCD |
| knob chain | 107 | DKTHTCPPCPAPEAAGGPSVFLFPPKPKDTLMISRTPEVT |
| (P1AJ0700, | | CVVVDVSHEDPEVKFNWYVDGVEVHNAKTKPREEQYN |
| P1AJ0744, | | STYRVVSVLTVLHQDWLNGKEYKCKVSNKALGAPIEKT |
| P1AJ0747) | | ISKAKGQPREPQVYTLPPCRDELTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSPGGGSG |
| | | GGGSGGEVQLLESGGGLVQPGGSLRLSCAASGFTFSSYA |
| | | MSWVRQAPGKGLEWVSAIIGSGASTYYADSVKGRFTIS |
| | | RDNSKNTLYLQMNSLRAEDTAVYYCAKGWFGGFNYW |
| | | GQGTLVTVSSASTKGPSVFPLAPSSKSTSGGTAALGCLV |
| | | EDYFPEPVTVSWNSGALTSGVHTFPAVLQSSGLYSLSSV |
| | | VTVPSSSLGTQTYICNVNHKPSNTKVDEKVEPKSCD |
| hole chain | 108 | QVQLVQSGAEVKKPGASVKVSCKASGYTLTDYNMDW |
| (P1AJ0700, | | VRQAPGQGLEWIGDIYPNTGGTIYNQKFKGRVTMTIDTS |
| P1AJ0747) | | TSTVYMELSSLRSEDTAVYYCTRFRGIHYAMDYWGQGT |
| | | TVTVSSASVAAPSVFIFPPSDEQLKSGTASVVCLLNNFYP |
| | | REAKVQWKVDNALQSGNSQESVTEQDSKDSTYSLSSTL |
| | | TLSKADYEKHKVYACEVTHQGLSSPVTKSFNRGECDKT |
| | | HTCPPCPAPEAAGGPSVFLFPPKPKDTLMASRTPEVTCV |
| | | VVDVSHEDPEVKFNWYVDGVEVHNAKTKPREEQYNST |
| | | YRVVSVLTVLAQDWLNGKEYKCKVSNKALGAPIEKTIS |
| | | KAKGQPREPQVCTLPPSRDELTKNQVSLSCAVKGFYPSD |
| | | IAVEWESNGQPENNYKTTPPVLDSDGSFFLVSKLTVDKS |
| | | RWQQGNVFSCSVMHEALHNAYTQKSLSLSPG |
| P1AJ0685 LC1 | 109 | QVQLQQSGGGLVQAGGSLRLSCTASGSTFSFSNYHMGW |
| | | YRQAPGKQRERVASISSRDSTYYSDSVKGRFTISRDTAR |
| | | NTVYLQMNSLEPEETAVYYCNARGRATGRDYWGQGTQ |
| | | VTVSSGGSGGGGSGGGGSGGGGSGGGGSGGQVQLVQS |
| | | GAEVKKPGASVKVSCKASGYTLTDYNMDWVRQAPGQ |
| | | GLEWIGDIYPNTGGTIYNQKFKGRVTMTIDTSTSTVYME |
| | | LSSLRSEDTAVYYCTRFRGIHYAMDYWGQGTTVTVSSA |
| | | STKGPSVFPLAPSSKSTSGGTAALGCLVEDYFPEPVTVS |
| | | WNSGALTSGVHTFPAVLQSSGLYSLSSVVTVPSSSLGTQ |
| | | TYICNVNHKPSNTKVDEKVEPKSCD |
| P1AJ0685 LC2 | 110 | QVQLQEFGGGLVQAGEALRLSCVASKTIFNTMPMGWY |
| | | RQAPGKERELVATITSSGVVNSADSVKGRFTISRDSAKR |
| | | TAYLQMNNLKPEDTAVYYCAAMFKPGIPEYWGRGTQV |
| | | TVSSGGSGGGGSGGGGSGGGGSGGGGSGGEIVLTQSPG |
| | | TLSLSPGERATLSCRASQSVTSSYLAWYQQKPGQAPRLL |
| | | INVGSRRATGIPDRFSGSGSGTDFTLTISRLEPEDFAVYYC |
| | | QQGIMLPPTFGQGTKVEIKSSASTKGPSVFPLAPSSKSTS |
| | | GGTAALGCLVKDYFPEPVTVSWNSGALTSGVHTFPAVL |
| | | QSSGLYSLSSVVTVPSSSLGTQTYICNVNHKPSNTKVDK |
| | | KVEPKSCD |
| P1AJ0744 LC1 | 111 | QVQLQESGGGLVQAGGSLRLSCTASGAYFIMGWFRQAP |
| | | GKQRELVARTIRDGTTDYADSVKGRFTISRNTAENTAYL |
| | | QMNSLKPEDTGVYYCAAGPLSKSFAPWDPAYWGQGTL |
| | | VTVSSGGSGGEIVLTQSPGTLSLSPGERATLSCRASQSVT |
| | | SSYLAWYQQKPGQAPRLLINVGSRRATGIPDRFSGSGSG |
| | | TDFTLTISRLEPEDFAVYYCQQGIMLPPTFGQGTKVEIKR |
| | | TVAAPSVFIFPPSDRKLKSGTASVVCLLNNFYPREAKVQ |
| | | WKVDNALQSGNSQESVTEQDSKDSTYSLSSTLTLSKAD |
| | | YEKHKVYACEVTHQGLSSPVTKSFNRGEC |
| P1AJ0744 LC2 | 112 | QVQLQQFGGGLVQPGGSLRLSCAVSRSIFSIDAMAWYR |
| | | QAPGKQRNLVGTITSDGTTNYVDSAKGRFTISRDNAKNT |
| | | VYLQMNSLKPEDTAVYFCNAGAVSRTGGYRPSGYWGQ |
| | | GTLVTVSSGGSGGQVQLVQSGAEVKKPGASVKVSCKAS |
| | | GYTLTDYNMDWVRQAPGQGLEWIGDIYPNTGGTIYNQ |
| | | KFKGRVTMTIDTSTSTVYMELSSLRSEDTAVYYCTRFRG |
| | | IHYAMDYWGQGTTVTVSSASVAAPSVFIFPPSDEQLKSG |
| | | TASVVCLLNNFYPREAKVQWKVDNALQSGNSQESVTE |
| | | QDSKDSTYSLSSTLTLSKADYEKHKVYACEVTHQGLSSP |
| | | VTKSFNRGEC |
| P1AJ0744 hole chain | 113 | EIVLTQSPATLSLSPGERATLSCRASESVDNYGLSFINWF |
| | | QQKPGQAPRLLIYGTSNRGSGIPARFSGSGSGTDFTLTISS |
| | | LEPEDFAVYFCQQSNEVPYTFGGGTKVEIKSSASTKGPS |
| | | VFPLAPSSKSTSGGTAALGCLVKDYFPEPVTVSWNSGAL |
| | | TSGVHTFPAVLQSSGLYSLSSVVTVPSSSLGTQTYICNVN |
| | | HKPSNTKVDKKVEPKSCDKTHTCPPCPAPEAAGGPSVFL |
| | | FPPKPKDTLMASRTPEVTCVVVDVSHEDPEVKFNWYVD |
| | | GVEVHNAKTKPREEQYNSTYRVVSVLTVLAQDWLNGK |
| | | EYKCKVSNKALGAPIEKTISKAKGQPREPQVCTLPPSRD |
| | | ELTKNQVSLSCAVKGFYPSDIAVEWESNGQPENNYKTTP |
| | | PVLDSDGSFFLVSKLTVDKSRWQQGNVFSCSVMHEALH |
| | | NAYTQKSLSLSPG |
| P1AJ0735 LC1 | 114 | QVQLQQFGGGLVQPGGSLRLSCAVSRSIFSIDAMAWYR |
| | | QAPGKQRNLVGTITSDGTTNYVDSAKGRFTISRDNAKNT |
| | | VYLQMNSLKPEDTAVYFCNAGAVSRTGGYRPSGYWGQ |
| | | GTLVTVSSGGSGGGGSGGGGSGGQVQLVQSGAEVKKP |
| | | GASVKVSCKASGYTLTDYNMDWVRQAPGQGLEWIGDI |
| | | YPNTGGTIYNQKFKGRVTMTIDTSTSTVYMELSSLRSED |
| | | TAVYYCTRFRGIHYAMDYWGQGTTVTVSSASTKGPSVF |
| | | PLAPSSKSTSGGTAALGCLVEDYFPEPVTVSWNSGALTS |
| | | GVHTFPAVLQSSGLYSLSSVVTVPSSSLGTQTYICNVNH |
| | | KPSNTKVDEKVEPKSCD |
| P1AJ0735 LC2 | 115 | QVQLQESGGGLVQAGGSLRLSCTASGAYFIMGWFRQAP |
| | | GKQRELVARTIRDGTTDYADSVKGRFTISRNTAENTAYL |
| | | QMNSLKPEDTGVYYCAAGPLSKSFAPWDPAYWGQGTL |
| | | VTVSSGGSGGGGSGGGGSGGEIVLTQSPGTLSLSPGERA |
| | | TLSCRASQSVTSSYLAWYQQKPGQAPRLLINVGSRRATG |
| | | IPDRFSGSGSGTDFTLTISRLEPEDFAVYYCQQGIMLPPTF |
| | | GQGTKVEIKSSASTKGPSVFPLAPSSKSTSGGTAALGCLV |
| | | KDYFPEPVTVSWNSGALTSGVHTFPAVLQSSGLYSLSSV |
| | | VTVPSSSLGTQTYICNVNHKPSNTKVDKKVEPKSCD |
| P1AJ0747 LC1 | 116 | QVQLQQFGGGLVQPGGSLRLSCAVSRSIFSIDAMAWYR |
| | | QAPGKQRNLVGTITSDGTTNYVDSAKGRFTISRDNAKNT |
| | | VYLQMNSLKPEDTAVYFCNAGAVSRTGGYRPSGYWGQ |
| | | GTLVTVSSGGSGGEIVLTQSPGTLSLSPGERATLSCRASQ |
| | | SVTSSYLAWYQQKPGQAPRLLINVGSRRATGIPDRFSGS |
| | | GSGTDFTLTISRLEPEDFAVYYCQQGIMLPPTFGQGTKVE |
| | | IKRTVAAPSVFIFPPSDRKLKSGTASVVCLLNNFYPREAK |
| | | VQWKVDNALQSGNSQESVTEQDSKDSTYSLSSTLTLSK |
| | | ADYEKHKVYACEVTHQGLSSPVTKSFNRGEC |
| P1AJ0747 LC2 | 117 | QVQLQESGGGLVQAGGSLRLSCTASGAYFIMGWFRQAP |
| | | GKQRELVARTIRDGTTDYADSVKGRFTISRNTAENTAYL |
| | | QMNSLKPEDTGVYYCAAGPLSKSFAPWDPAYWGQGTL |
| | | VTVSSGGSGGEIVLTQSPATLSLSPGERATLSCRASESVD |
| | | NYGLSFINWFQQKPGQAPRLLIYGTSNRGSGIPARFSGSG |
| | | SGTDFTLTISSLEPEDFAVYFCQQSNEVPYTFGGGTKVEI |
| | | KSSASTKGPSVFPLAPSSKSTSGGTAALGCLVKDYFPEPV |
| | | TVSWNSGALTSGVHTFPAVLQSSGLYSLSSVVTVPSSSL |
| | | GTQTYICNVNHKPSNTKVDKKVEPKSCD |
| (G4S)₁ peptide linker | 118 | GGGGS |
| (G4S)₂ peptide linker | 119 | GGGGSGGGGS |
| (G4S)₃ peptide linker | 120 | GGGGSGGGGSGGGGS |
| (G4S)₅ peptide linker | 121 | GGGGSGGGGSGGGGSGGGGSGGGGS |
| LC1 (P1AI5057, | 122 | QVQLQQSGGGLVQPGGSLTLSCVASGSILEMSRMAWYR |
| P1AM4293) | | QAPGKMRELVASITSIGSIVYADSVKGRFTLSRDNTKNT |
| | | VYLQMNSLKPEDTAVYRCNLLGTWSDETYWGQGTQVT |
| | | VSSGGSGGGGSGGGGSGGGGSGGGGSGGDIVMTQSPDS |
| | | LAVSLGERATINCKASESVDTSDNSFIHWYQQKPGQSPK |
| | | LLIYRSSTLESGVPDRFSGSGSGTDFTLTISSLQAEDVAV |
| | | YYCQQNYDVPWTFGQGTKVEIKRTVAAPSVFIFPPSDRK |
| | | LKSGTASVVCLLNNFYPREAKVQWKVDNALQSGNSQES |
| | | VTEQDSKDSTYSLSSTLTLSKADYEKHKVYACEVTHQG |
| | | LSSPVTKSFNRGEC |
| knob chain | 123 | DKTHTCPPCPAPEAAGGPSVFLFPPKPKDTLMISRTPEVT |
| (P1AI5057, | | CVVVDVSHEDPEVKFNWYVDGVEVHNAKTKPREEQYN |
| P1AM4293) | | STYRVVSVLTVLHQDWLNGKEYKCKVSNKALGAPIEKT |
| | | ISKAKGQPREPQVYTLPPCRDELTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSPGGGSG |
| | | GGGSGGEVQLLESGGGLVQPGGSLRLSCAASGFSFSSYT |
| | | MSWVRQAPGKGLEWVATISGGGRDIYYPDSVKGRFTIS |
| | | RDNSKNTLYLQMNSLRAEDTAVYYCVLLTGRVYFALDS |
| | | WGQGTLVTVSSASTKGPSVFPLAPSSKSTSGGTAALGCL |
| | | VEDYFPEPVTVSWNSGALTSGVHTFPAVLQSSGLYSLSS |
| | | VVTVPSSSLGTQTYICNVNHKPSNTKVDEKVEPKSC |
| hole chain (P1AI5057, | 124 | DIQMTQSPSSLSASVGDRVTITCRASQSISSYLNWYQQKP |
| P1AM4294) | | GKAPKLLIYTASSLQSGVPSRFSGSGSGTDFTLTISSLQPE |
| | | DFATYYCQQSYSTPLTFGGGTKVEIKSSASTKGPSVFPLA |
| | | PSSKSTSGGTAALGCLVKDYFPEPVTVSWNSGALTSGVH |
| | | TFPAVLQSSGLYSLSSVVTVPSSSLGTQTYICNVNHKPSN |
| | | TKVDKKVEPKSCDKTHTCPPCPAPEAAGGPSVFLFPPKP |
| | | KDTLMISRTPEVTCVVVDVSHEDPEVKFNWYVDGVEVH |
| | | NAKTKPREEQYNSTYRVVSVLTVLHQDWLNGKEYKCK |
| | | VSNKALGAPIEKTISKAKGQPREPQVCTLPPSRDELTKNQ |
| | | VSLSCAVKGFYPSDIAVEWESNGQPENNYKTTPPVLDSD |
| | | GSFFLVSKLTVDKSRWQQGNVFSCSVMHEALHNHYTQ |
| | | KSLSLSPG |
| LC2 (P1AI5057, | 125 | QVQLQESGGGLVQAGGSLRLSCAASISISKIDLMGWYRQ |
| P1AM4294) | | APGRQRELVARVINGGDSYYSTSVKGRFTISRDNAKNT |
| | | LYLQMNSLKPEDTAVYYCYGVPESLAAFHWGQGTLVT |
| | | VSSGGSGGGGSGGGGSGGGGSGGGGSGGEVQLLESGG |
| | | GLVQPGGSLRLSCAASGFTFSSYAMSWVRQAPGKGLEW |
| | | VSAITGSGGSTYYADSVKGRFTISRDNSRNTLYLQMNSL |
| | | RAEDTAVYYCAKGEGYAGSSYFRASDIWGQGTMVTVS |
| | | SASVAAPSVFIFPPSDEQLKSGTASVVCLLNNFYPREAKV |
| | | QWKVDNALQSGNSQESVTEQDSKDSTYSLSSTLTLSKA |
| | | DYEKHKVYACEVTHQGLSSPVTKSFNRGEC |
| hole chain P1AM4293 | 126 | DIVMTQSPDSLAVSLGERATINCKASESVDTSDNSFIHW |
| | | YQQKPGQSPKLLIYRSSTLESGVPDRFSGSGSGTDFTLTIS |
| | | SLQAEDVAVYYCQQNYDVPWTFGQGTKVEIKSSASTKG |
| | | PSVFPLAPSSKSTSGGTAALGCLVKDYFPEPVTVSWNSG |
| | | ALTSGVHTFPAVLQSSGLYSLSSVVTVPSSSLGTQTYICN |
| | | VNHKPSNTKVDKKVEPKSCDKTHTCPPCPAPEAAGGPS |
| | | VFLFPPKPKDTLMISRTPEVTCVVVDVSHEDPEVKFNWY |
| | | VDGVEVHNAKTKPREEQYNSTYRVVSVLTVLHQDWLN |
| | | GKEYKCKVSNKALGAPIEKTISKAKGQPREPQVCTLPPS |
| | | RDELTKNQVSLSCAVKGFYPSDIAVEWESNGQPENNYK |
| | | TTPPVLDSDGSFFLVSKLTVDKSRWQQGNVFSCSVMHE |
| | | ALHNHYTQKSLSLSPG |
| LC2 P1AM4293 | 127 | QVQLQESGGGLVQAGGSLRLSCAASISISKIDLMGWYRQ |
| | | APGRQRELVARVTNGGDSYYSTSVKGRFTISRDNAKNT |
| | | LYLQMNSLKPEDTAVYYCYGVPESLAAFHWGQGTLVT |
| | | VSSGGSGGGGSGGGGSGGGGSGGGGSGGEVQLLESGG |
| | | GLVQPGGSLRLSCAASGFSFSSYTMSWVRQAPGKGLEW |
| | | VATISGGGRDIYYPDSVKGRFTISRDNSKNTLYLQMNSL |
| | | RAEDTAVYYCVLLTGRVYFALDSWGQGTLVTVSSASV |
| | | AAPSVFIFPPSDEQLKSGTASVVCLLNNFYPREAKVQWK |
| | | VDNALQSGNSQESVTEQDSKDSTYSLSSTLTLSKADYEK |
| | | HKVYACEVTHQGLSSPVTKSFNRGEC |
| knob chain | 128 | DKTHTCPPCPAPEAAGGPSVFLFPPKPKDTLMISRTPEVT |
| P1AM4294 | | CVVVDVSHEDPEVKFNWYVDGVEVHNAKTKPREEQYN |
| | | STYRVVSVLTVLHQDWLNGKEYKCKVSNKALGAPIEKT |
| | | ISKAKGQPREPQVYTLPPCRDELTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSPGGGSG |
| | | GGGSGGEVQLLESGGGLVQPGGSLRLSCAASGFTFSSYA |
| | | MSWVRQAPGKGLEWVSAITGSGGSTYYADSVKGRFTIS |
| | | RDNSRNTLYLQMNSLRAEDTAVYYCAKGEGYAGSSYF |
| | | RASDIWGQGTMVTVSSASTKGPSVFPLAPSSKSTSGGTA |
| | | ALGCLVEDYFPEPVTVSWNSGALTSGVHTFPAVLQSSGL |
| | | YSLSSVVTVPSSSLGTQTYICNVNHKPSNTKVDEKVEPK |
| | | SC |
| LC1 P1AM4294 | 129 | QVQLQQSGGGLVQPGGSLTLSCVASGSILEMSRMAWYR |
| | | QAPGKMRELVASITSIGSIVYADSVKGRFTLSRDNTKNT |
| | | VYLQMNSLKPEDTAVYRCNLLGTWSDETYWGQGTQVT |
| | | VSSGGSGGGGSGGGGSGGGGSGGGGSGGDIQMTQSPSS |
| | | LSASVGDRVTITCRASQSISSYLNWYQQKPGKAPKLLIY |
| | | TASSLQSGVPSRFSGSGSGTDFTLTISSLQPEDFATYYCQ |
| | | QSYSTPLTFGGGTKVEIKRTVAAPSVFIFPPSDRKLKSGT |
| | | ASVVCLLNNFYPREAKVQWKVDNALQSGNSQESVTEQ |
| | | DSKDSTYSLSSTLTLSKADYEKHKVYACEVTHQGLSSPV |
| | | TKSFNRGEC |
| P1AK2599 knob | 130 | QVQLQESGGGLVQAGGSLRLSCAASISISKIDLMGWYRQ |
| chain | | APGRQRELVARVINGGDSYYSTSVKGRFTISRDNAKNT |
| | | LYLQMNSLKPEDTAVYYCYGVPESLAAFHWGQGTLVT |
| | | VSSGGSGGGGSGGGGSGGGGSGGGGSGGEVQLLESGG |
| | | GLVQPGGSLRLSCAASGFTFSSYAMSWVRQAPGKGLEW |
| | | VSAITGSGGSTYYADSVKGRFTISRDNSRNTLYLQMNSL |
| | | RAEDTAVYYCAKGEGYAGSSYFRASDIWGQGTMVTVS |
| | | SASTKGPSVFPLAPSSKSTSGGTAALGCLVKDYFPEPVTV |
| | | SWNSGALTSGVHTFPAVLQSSGLYSLSSVVTVPSSSLGT |
| | | QTYICNVNHKPSNTKVDKKVEPKSCDKTHTCPPCPAPEA |
| | | AGGPSVFLFPPKPKDTLMISRTPEVTCVVVDVSHEDPEV |
| | | KFNWYVDGVEVHNAKTKPREEQYNSTYRVVSVLTVLH |
| | | QDWLNGKEYKCKVSNKALGAPIEKTISKAKGQPREPQV |
| | | YTLPPCRDELTKNQVSLWCLVKGFYPSDIAVEWESNGQ |
| | | PENNYKTTPPVLDSDGSFFLYSKLTVDKSRWQQGNVFS |
| | | CSVMHEALHNHYTQKSLSLSPG |
| P1AK2798 LC | 131 | QVQLQQSGGGLVQPGGSLTLSCVASGSILEMSRMAWYR |
| | | QAPGKMRELVASITSIGSIVYADSVKGRFTLSRDNTKNT |
| | | VYLQMNSLKPEDTAVYRCNLLGTWSDETYWGQGTQVT |
| | | VSSGGSGGGGSGGGGSGGGGSGGGGSGGDIQMTQSPSS |
| | | LSASVGDRVTITCRASQSISSYLNWYQQKPGKAPKLLIY |
| | | TASSLQSGVPSRFSGSGSGTDFTLTISSLQPEDFATYYCQ |
| | | QSYSTPLTFGGGTKVEIKRTVAAPSVFIFPPSDEQLKSGT |
| | | ASVVCLLNNFYPREAKVQWKVDNALQSGNSQESVTEQ |
| | | DSKDSTYSLSSTLTLSKADYEKHKVYACEVTHQGLSSPV |
| | | TKSFNRGEC |
| P1AK2799 LC | 132 | QVQLQESGGGLVQAGGSLRLSCAASISISKIDLMGWYRQ |
| | | APGRQRELVARVINGGDSYYSTSVKGRFTISRDNAKNT |
| | | LYLQMNSLKPEDTAVYYCYGVPESLAAFHWGQGTLVT |
| | | VSSGGSGGGGGGGGSGGGGSGGGGSGGDIQMTQSPSS |
| | | LSASVGDRVTITCRASQSISSYLNWYQQKPGKAPKLLIY |
| | | TASSLQSGVPSRFSGSGSGTDFTLTISSLQPEDFATYYCQ |
| | | QSYSTPLTFGGGTKVEIKRTVAAPSVFIFPPSDEQLKSGT |
| | | ASVVCLLNNFYPREAKVQWKVDNALQSGNSQESVTEQ |
| | | DSKDSTYSLSSTLTLSKADYEKHKVYACEVTHQGLSSPV |
| | | TKSFNRGEC |
| P1AK2802 LC | 133 | QVQLQQSGGGLVQPGGSLTLSCVASGSILEMSRMAWYR |
| | | QAPGKMRELVASITSIGSIVYADSVKGRFTLSRDNTKNT |
| | | VYLQMNSLKPEDTAVYRCNLLGTWSDETYWGQGTQVT |
| | | VSSGGSGGGGSGGGGSGGGGSGGGGSGGDIQMTQSPSS |
| | | LSASVGDRVTITCRASQGISSWLAWYQQKPGKAPKLLIY |
| | | EASSLQSGVPSRFSGSGSGTDFTLTISSLQPEDFATYYCQ |
| | | QANQFPFTFGPGTKVDIKRTVAAPSVFIFPPSDEQLKSGT |
| | | ASVVCLLNNFYPREAKVQWKVDNALQSGNSQESVTEQ |
| | | DSKDSTYSLSSTLTLSKADYEKHKVYACEVTHQGLSSPV |
| | | TKSFNRGEC |
| P1AK2802, | 134 | EVQLLESGGGLVQPGGSLRLSCAASGFTFSSYWMSWVR |
| P1AK2803 knob | | QAPGKGLEWVSAISGSGGSRYYAESVKGRFTISRDNSKN |
| chain | | TLYLQMNSLRAEDTAVYYCASSPLQWIDVWGQGTTVT |
| | | VSSASTKGPSVFPLAPSSKSTSGGTAALGCLVKDYFPEPV |
| | | TVSWNSGALTSGVHTFPAVLQSSGLYSLSSVVTVPSSSL |
| | | GTQTYICNVNHKPSNTKVDKKVEPKSCDKTHTCPPCPAP |
| | | EAAGGPSVFLFPPKPKDTLMISRTPEVTCVVVDVSHEDP |
| | | EVKFNWYVDGVEVHNAKTKPREEQYNSTYRVVSVLTV |
| | | LHQDWLNGKEYKCKVSNKALGAPIEKTISKAKGQPREP |
| | | QVYTLPPCRDELTKNQVSLWCLVKGFYPSDIAVEWESN |
| | | GQPENNYKTTPPVLDSDGSFFLYSKLTVDKSRWQQGNV |
| | | FSCSVMHEALHNHYTQKSLSLSPG |
| P1AK2803 LC | 135 | QVQLQESGGGLVQAGGSLRLSCAASISISKIDLMGWYRQ |
| | | APGRQRELVARVTNGGDSYYSTSVKGRFTISRDNAKNT |
| | | LYLQMNSLKPEDTAVYYCYGVPESLAAFHWGQGTLVT |
| | | VSSGGSGGGGSGGGGSGGGGSGGGGSGGDIQMTQSPSS |
| | | LSASVGDRVTITCRASQGISSWLAWYQQKPGKAPKLLIY |
| | | EASSLQSGVPSRFSGSGSGTDFTLTISSLQPEDFATYYCQ |
| | | QANQFPFTFGPGTKVDIKRTVAAPSVFIFPPSDEQLKSGT |
| | | ASVVCLLNNFYPREAKVQWKVDNALQSGNSQESVTEQ |
| | | DSKDSTYSLSSTLTLSKADYEKHKVYACEVTHQGLSSPV |
| | | TKSFNRGEC |
| P1AK2806 knob | 136 | QVQLQQSGGGLVQPGGSLTLSCVASGSILEMSRMAWYR |
| chain | | QAPGKMRELVASITSIGSIVYADSVKGRFTLSRDNTKNT |
| | | VYLQMNSLKPEDTAVYRCNLLGTWSDETYWGQGTQVT |
| | | VSSGGSGGGGSGGGGSGGGGSGGGGSGGEVQLLESGG |
| | | GLVQPGGSLRLSCAASGFTFSSYAMSWVRQAPGKGLEW |
| | | VSAITGSGGSTYYADSVKGRFTISRDNSRNTLYLQMNSL |
| | | RAEDTAVYYCAKGEGYAGSSYFRASDIWGQGTMVTVS |
| | | SASTKGPSVFPLAPSSKSTSGGTAALGCLVKDYFPEPVTV |
| | | SWNSGALTSGVHTFPAVLQSSGLYSLSSVVTVPSSSLGT |
| | | QTYICNVNHKPSNTKVDKKVEPKSCDKTHTCPPCPAPEA |
| | | AGGPSVFLFPPKPKDTLMISRTPEVTCVVVDVSHEDPEV |
| | | KFNWYVDGVEVHNAKTKPREEQYNSTYRVVSVLTVLH |
| | | QDWLNGKEYKCKVSNKALGAPIEKTISKAKGQPREPQV |
| | | YTLPPCRDELTKNQVSLWCLVKGFYPSDIAVEWESNGQ |
| | | PENNYKTTPPVLDSDGSFFLYSKLTVDKSRWQQGNVFS |
| | | CSVMHEALHNHYTQKSLSLSPG |
| P1AK2809, | 137 | DIQMTQSPSSLSASVGDRVTITCRASQGISSWLAWYQQK |
| P1AK2810 LC | | PGKAPKLLIYEASSLQSGVPSRFSGSGSGTDFTLTISSLQP |
| | | EDFATYYCQQANQFPFTFGPGTKVDIKRTVAAPSVFIFPP |
| | | SDEQLKSGTASVVCLLNNFYPREAKVQWKVDNALQSG |
| | | NSQESVTEQDSKDSTYSLSSTLTLSKADYEKHKVYACEV |
| | | THQGLSSPVTKSFNRGEC |
| P1AK2809 knob | 138 | QVQLQQSGGGLVQPGGSLTLSCVASGSILEMSRMAWYR |
| chain | | QAPGKMRELVASITSIGSIVYADSVKGRFTLSRDNTKNT |
| | | VYLQMNSLKPEDTAVYRCNLLGTWSDETYWGQGTQVT |
| | | VSSGGSGGGGSGGGGSGGGGSGGGGSGGEVQLLESGG |
| | | GLVQPGGSLRLSCAASGFTFSSYWMSWVRQAPGKGLE |
| | | WVSAISGSGGSRYYAESVKGRFTISRDNSKNTLYLQMNS |
| | | LRAEDTAVYYCASSPLQWIDVWGQGTTVTVSSASTKGP |
| | | SVFPLAPSSKSTSGGTAALGCLVKDYFPEPVTVSWNSGA |
| | | LTSGVHTFPAVLQSSGLYSLSSVVTVPSSSLGTQTYICNV |
| | | NHKPSNTKVDKKVEPKSCDKTHTCPPCPAPEAAGGPSVF |
| | | LFPPKPKDTLMISRTPEVTCVVVDVSHEDPEVKFNWYV |
| | | DGVEVHNAKTKPREEQYNSTYRVVSVLTVLHQDWLNG |
| | | KEYKCKVSNKALGAPIEKTISKAKGQPREPQVYTLPPCR |
| | | DELTKNQVSLWCLVKGFYPSDIAVEWESNGQPENNYKT |
| | | TPPVLDSDGSFFLYSKLTVDKSRWQQGNVFSCSVMHEA |
| | | LHNHYTQKSLSLSPG |
| P1AK2810 knob | 139 | QVQLQESGGGLVQAGGSLRLSCAASISISKIDLMGWYRQ |
| chain | | APGRQRELVARVTNGGDSYYSTSVKGRFTISRDNAKNT |
| | | LYLQMNSLKPEDTAVYYCYGVPESLAAFHWGQGTLVT |
| | | VSSGGSGGGGSGGGGSGGGGSGGGGSGGEVQLLESGG |
| | | GLVQPGGSLRLSCAASGFTFSSYWMSWVRQAPGKGLE |
| | | WVSAISGSGGSRYYAESVKGRFTISRDNSKNTLYLQMNS |
| | | LRAEDTAVYYCASSPLQWIDVWGQGTTVTVSSASTKGP |
| | | SVFPLAPSSKSTSGGTAALGCLVKDYFPEPVTVSWNSGA |
| | | LTSGVHTFPAVLQSSGLYSLSSVVTVPSSSLGTQTYICNV |
| | | NHKPSNTKVDKKVEPKSCDKTHTCPPCPAPEAAGGPSVF |
| | | LFPPKPKDTLMISRTPEVTCVVVDVSHEDPEVKFNWYV |
| | | DGVEVHNAKTKPREEQYNSTYRVVSVLTVLHQDWLNG |
| | | KEYKCKVSNKALGAPIEKTISKAKGQPREPQVYTLPPCR |
| | | DELTKNQVSLWCLVKGFYPSDIAVEWESNGQPENNYKT |
| | | TPPVLDSDGSFFLYSKLTVDKSRWQQGNVFSCSVMHEA |
| | | LHNHYTQKSLSLSPG |
| PD-1 binder 3 7G12 | 140 | EVQLLESGGGLVQPGGSLRLSCAASGFTFSSYWMSWVR |
| VH | | QAPGKGLEWVSAISGSGGSRYYAESVKGRFTISRDNSKN |
| | | TLYLQMNSLRAEDTAVYYCASSPLQWIDVWGQGTTVT |
| | | VSS |
| PD-1 binder 3 7G12 | 141 | DIQMTQSPSSLSASVGDRVTITCRASQGISSWLAWYQQK |
| VL | | PGKAPKLLIYEASSLQSGVPSRFSGSGSGTDFTLTISSLQP |
| | | EDFATYYCQQANQFPFTFGPGTKVDIK |

1. An antigen binding molecule comprising
i) a first target-binding domain,
ii) a second target-binding domain,
iii) a first cytokine receptor-binding domain,
iv) a second cytokine receptor-binding domain, and
v) a Fc domain,
wherein the first target-binding domain is capable of binding a first epitope on the target antigen and the second target-binding domain is capable of binding a second epitope on the target antigen, wherein the first and the second target-binding domains do not compete for binding on the tumor-associated antigen; and
wherein the first cytokine receptor-binding domain is capable of binding a first cytokine receptor subunit and the second cytokine receptor-binding domain is capable of binding a second cytokine receptor subunit.

2. The antigen binding molecule according to claim 1, wherein first and second target-binding domains are antibody fragments, particularly Fv, Fab, scFv, scFab or single domain antibodies.

3. The antigen binding molecule according to claim 1, wherein the first and second target-binding domain are Fab molecules, wherein the first target-binding domain comprises a heavy chain variable domain (VH₁), a light chain variable domain (VL₁), a heavy chain constant domain (CH1₁) and a light chain constant domain (CL₁) and the second target-binding domain comprises a heavy chain variable domain (VH₂), a light chain variable domain (VL₂), a heavy chain constant domain (CH1₂) and a light chain constant domain (CL₂).

4. (canceled)

5. The antigen binding molecule according to claim 1, wherein the first target-binding domain and/or the second target-binding domain is a cross-Fab molecule.

6. The antigen binding molecule according to claim 1, wherein the first target-binding domain and/or the second target-binding domain comprise charge mutations.

7. The antigen binding molecule according to claim 1, wherein the first target-binding domain is a cross-Fab and the second target-binding domain comprises charge mutations or wherein the second target-binding domain is a cross-Fab and the first target-binding domain comprises charge mutations.

8. The antigen binding molecule according to claim 1, wherein the first target-binding domain and the second target-binding domain specifically bind to a tumor-associated antigen or a T cell antigen.

9. The antigen binding molecule according to claim 1, wherein the first target-binding domain and the second target-binding domain specifically bind to FAP, PD-1, Her2, Her3, LAG-3, CEA or EGFR.

10. The antigen binding molecule according to claim 1, wherein
a) the first target-binding domain comprises a VH₁ of SEQ ID NO: 20 and a VL₁ of SEQ ID NO: 21 and the second target-binding domain comprises a VH₂ of SEQ ID NO: 22 and a VL₂ of SEQ ID NO: 23, or
b) the first target-binding domain comprises a VH₁ of SEQ ID NO: 22 and a VL₁ of SEQ ID NO: 23 and the second target-binding domain comprises a VH₂ of SEQ ID NO: 20 and a VL₂ of SEQ ID NO: 21, or
c) the first target-binding domain comprises a VH₁ of SEQ ID NO: 80 and a VL₁ of SEQ ID NO: 81 and the second target-binding domain comprises a VH₂ of SEQ ID NO: 82 and a VL₂ of SEQ ID NO: 83, or
d) the first target-binding domain comprises a VH₁ of SEQ ID NO: 82 and a VL₁ of SEQ ID NO: 83 and the second target-binding domain comprises a VH₂ of SEQ ID NO: 80 and a VL₂ of SEQ ID NO: 81; or
e) the first target-binding domain comprises a VH₁ of SEQ ID NO: 82 and a VL₁ of SEQ ID NO: 83 and the second target-binding domain comprises a VH₂ of SEQ ID NO: 140 and a VL₂ of SEQ ID NO: 141; or
f) the first target-binding domain comprises a VH₁ of SEQ ID NO: 140 and a VL₁ of SEQ ID NO: 141 and the second target-binding domain comprises a VH₂ of SEQ ID NO: 82 and a VL₂ of SEQ ID NO: 83.

11. The antigen binding molecule according to claim 1, wherein both the first and second cytokine receptor subunits are subunits of the IFNγ receptor complex or IL-2 receptor complex.

12. The antigen binding molecule according to claim 1, wherein
a) the first cytokine receptor-binding domain is capable of binding IFNγR1 and the second cytokine receptor-binding domain is capable of binding IFNγR2, or
b) the first cytokine receptor-binding domain is capable of binding IFNγR2 and the second cytokine receptor-binding domain is capable of binding IFNγR1;
c) the first cytokine receptor-binding domain is capable of binding IL-2Rβ and the second cytokine receptor-binding domain is capable of binding IL-2Rγ; or
d) the first cytokine receptor-binding domain is capable of binding IL-2Rγ and the second cytokine receptor-binding domain is capable of binding IL-2Rβ.

13. The antigen binding molecule according to claim 1, wherein the first and second cytokine receptor-binding domains are antibody fragments, particularly Fv, Fab, scFv, scFab, single domain antibodies or VHH domains.

14. (canceled)

15. The antigen binding molecule according to claim 1, wherein
a) the first cytokine receptor-binding domain comprises an amino acid sequence selected from SEQ ID NO: 1, SEQ ID NO: 2, SEQ ID NO: 3 and SEQ ID NO: 5, and the second cytokine receptor-binding domain comprises an amino acid sequence selected from SEQ ID NO: 6, SEQ ID NO: 7 and SEQ ID NO: 9, or
b) the first cytokine receptor-binding domain comprises an amino acid sequence selected from SEQ ID NO: 6, SEQ ID NO: 7 and SEQ ID NO: 9, and the second cytokine receptor-binding domain comprises an amino acid sequence selected from SEQ ID NO: 1, SEQ ID NO: 2, SEQ ID NO: 3 and SEQ ID NO: 5; or
c) the first cytokine receptor-binding domain comprises an amino acid sequence selected from SEQ ID NO: 60, SEQ ID NO: 61, SEQ ID NO: 62 and SEQ ID NO: 64, and the second cytokine receptor-binding domain comprises an amino acid sequence selected from SEQ ID NO: 66, SEQ ID NO: 67, SEQ ID NO: 68 and SEQ ID NO: 69, or
d) the first cytokine receptor-binding domain comprises an amino acid sequence selected from SEQ ID NO: 66, SEQ ID NO: 67, SEQ ID NO: 68 and SEQ ID NO: 69, and the second cytokine receptor-binding domain comprises an amino acid sequence selected from SEQ ID NO: 60, SEQ ID NO: 61, SEQ ID NO: 62 and SEQ ID NO: 64.

16. The antigen binding molecule according to claim 1, wherein the Fc domain comprises a first Fc domain subunit and a second Fc domain subunit.

17. The antigen binding molecule according to claim 1, wherein the Fc domain is an IgG, particularly an IgG₁, Fc domain.

18. The antigen binding molecule according to claim 1, wherein the Fc domain is a human Fc domain.

19. The antigen binding molecule according to claim 1, wherein the Fc domain comprises a modification promoting the association of the first and the second subunit of the Fc domain.

20. The antigen binding molecule according to claim 1, wherein the Fc domain comprises one or more amino acid substitution that reduces binding to an Fc receptor and/or effector function.

21. The antigen binding molecule according to claim 1, wherein
the first cytokine receptor-binding domain is fused at its C-terminus to the N-terminus of VH₁ or VL₁ of the first target-binding domain, and the first target-binding domain is fused at its N-terminus of VH₁ or VL₁ to the C-terminus of the first Fc domain subunit, and the second cytokine receptor-binding domain is fused at its C-terminus to the N-terminus of VH₂ or VL₂ of the second target-binding domain, and the second target-binding domain is fused at its C-terminus of CH1₂ or CL₁ to the N-terminus of the second Fc domain subunit.

22. The antigen binding molecule according to claim 1, comprising
a) a first polypeptide comprising in order from the N-terminus to C-terminus a first Fc domain subunit, a VH₁ and a CH1₁, a second polypeptide comprising in order from the N-terminus to C-terminus a the first cytokine receptor-binding domain, a VL₁ and a CL₁, a third polypeptide comprising in order from the N-terminus to C-terminus a VL₂, a CH1₂ and a second Fc domain subunit, and a fourth polypeptide comprising in order from the N-terminus to C-terminus a the second cytokine receptor-binding domain, a VH₂ and a CL₂; or
b) a first polypeptide comprising in order from the N-terminus to C-terminus a first Fc domain subunit, a VL₁ and a CH1₁, a second polypeptide comprising in order from the N-terminus to C-terminus a the first cytokine receptor-binding domain, a VH₁ and a CL₁, a third polypeptide comprising in order from the N-terminus to C-terminus a VH₂, a CH1₂ and a second Fc domain subunit, and a fourth polypeptide comprising in order from the N-terminus to C-terminus a the second cytokine receptor-binding domain, a VL₂ and a CL₂; or
c) a first polypeptide comprising in order from the N-terminus to C-terminus a first Fc domain subunit, a VL₁ and a CL₁, a second polypeptide comprising in order from the N-terminus to C-terminus a the first cytokine receptor-binding domain, a VH₁ and a CH1₁, a third polypeptide comprising in order from the N-terminus to C-terminus a VL₂, a CH1₂ and a second Fc domain subunit, and a fourth polypeptide comprising in order from the N-terminus to C-terminus a the second cytokine receptor-binding domain, a VH₂ and a CL₂; or
d) a first polypeptide comprising in order from the N-terminus to C-terminus a first Fc domain subunit, a VH₁ and a CL₁, a second polypeptide comprising in order from the N-terminus to C-terminus a the first cytokine receptor-binding domain, a VL₁ and a CH1₁, a third polypeptide comprising in order from the N-terminus to C-terminus a VH₂, a CH1₂ and a second Fc domain subunit, and a fourth polypeptide comprising in order from the N-terminus to C-terminus a the second cytokine receptor-binding domain, a VL₂ and a CL₂; or
e) a first polypeptide comprising in order from the N-terminus to C-terminus a first Fc domain subunit, a VH₁ and a CH1₁, a second polypeptide comprising in order from the N-terminus to C-terminus a the first cytokine receptor-binding domain, a VL₁ and a CL₁, a third polypeptide comprising in order from the N-terminus to C-terminus a the second cytokine receptor-binding domain, a VL₂, a CH1₂ and a second Fc domain subunit, and a fourth polypeptide comprising in order from the N-terminus to C-terminus a VH₂ and a CL₂; or
f) a first polypeptide comprising in order from the N-terminus to C-terminus a first Fc domain subunit, a VL₁ and a CH1₁, a second polypeptide comprising in order from the N-terminus to C-terminus a the first cytokine receptor-binding domain, a VH₁ and a CL₁, a third polypeptide comprising in order from the N-terminus to C-terminus a the second cytokine receptor-binding domain, a VH₂, a CH1₂ and a second Fc domain subunit, and a fourth polypeptide comprising in order from the N-terminus to C-terminus a VL₂ and a CL₂; or
g) a first polypeptide comprising in order from the N-terminus to C-terminus a first Fc domain subunit, a VL₁ and a CL₁, a second polypeptide comprising in order from the N-terminus to C-terminus a the first cytokine receptor-binding domain, a VH₁ and a CH1₁, a third polypeptide comprising in order from the N-terminus to C-terminus a the second cytokine receptor-binding domain, a VL₂, a CH1₂ and a second Fc domain subunit, and a fourth polypeptide comprising in order from the N-terminus to C-terminus a VH₂ and a CL₂; or
h) a first polypeptide comprising in order from the N-terminus to C-terminus a first Fc domain subunit, a VH₁ and a CL₁, a second polypeptide comprising in order from the N-terminus to C-terminus a the first cytokine receptor-binding domain, a VL₁ and a CH1₁, a third polypeptide comprising in order from the N-terminus to C-terminus a the second cytokine receptor-binding domain, a VH₂, a CH1₂ and a second Fc domain subunit, and a fourth polypeptide comprising in order from the N-terminus to C-terminus a VL₂ and a CL₂;
wherein VH₁, VL₁, CH1₁, and CL₁ form the first target-binding domain and VH₂, VL₂, CH1₂, and CL₂ form the second target-binding domain.

23. The antigen binding molecule according to claim 1, wherein the first target-binding domain and the second target-binding domain specifically bind to FAP and the first and second cytokine receptor subunits are subunits of the IFNγ receptor complex.

24. The antigen binding molecule according to claim 1, comprising
a) a first polypeptide comprising the amino acid sequence of SEQ ID NO: 50, a second polypeptide comprising the amino acid sequence of SEQ ID NO: 48, a third polypeptide comprising the amino acid sequence of SEQ ID NO: 51 and a fourth polypeptide comprising the amino acid sequence of SEQ ID NO: 49; or
b) a first polypeptide comprising an amino acid sequence of SEQ ID NO: 99, a second polypeptide comprising an amino acid sequence of SEQ ID NO: 97, a third polypeptide comprising an amino acid sequence of SEQ ID NO: 100, and a fourth polypeptide comprising an amino acid sequence of SEQ ID NO: 98; or
c) a first polypeptide comprising an amino acid sequence of SEQ ID NO: 103, a second polypeptide comprising an amino acid sequence of SEQ ID NO: 101, a third polypeptide comprising an amino acid sequence of SEQ ID NO: 104, and a fourth polypeptide comprising an amino acid sequence of SEQ ID NO: 102; or
d) a first polypeptide comprising an amino acid sequence of SEQ ID NO: 107, a second polypeptide comprising an amino acid sequence of SEQ ID NO: 105, a third polypeptide comprising an amino acid sequence of SEQ ID NO: 108, and a fourth polypeptide comprising an amino acid sequence of SEQ ID NO: 106; or
e) a first polypeptide comprising an amino acid sequence of SEQ ID NO: 99, a second polypeptide comprising an amino acid sequence of SEQ ID NO: 109, a third polypeptide comprising an amino acid sequence of SEQ ID NO: 100, and a fourth polypeptide comprising an amino acid sequence of SEQ ID NO: 110; or
f) a first polypeptide comprising an amino acid sequence of SEQ ID NO: 107, a second polypeptide comprising an amino acid sequence of SEQ ID NO: 111, a third polypeptide comprising an amino acid sequence of SEQ ID NO: 113, and a fourth polypeptide comprising an amino acid sequence of SEQ ID NO: 112; or
g) a first polypeptide comprising an amino acid sequence of SEQ ID NO: 99, a second polypeptide comprising an amino acid sequence of SEQ ID NO: 114, a third polypeptide comprising an amino acid sequence of SEQ ID NO: 100, and a fourth polypeptide comprising an amino acid sequence of SEQ ID NO: 115; or
h) a first polypeptide comprising an amino acid sequence of SEQ ID NO: 107, a second polypeptide comprising an amino acid sequence of SEQ ID NO: 116, a third polypeptide comprising an amino acid sequence of SEQ ID NO: 108, and a fourth polypeptide comprising an amino acid sequence of SEQ ID NO: 117.

25. The antigen binding molecule according to claim 1, wherein the first target-binding domain and the second target-binding domain specifically bind to PD-1 and the first and second cytokine receptor subunits are subunits of the IL-2 receptor complex.

26. The antigen binding molecule according to claim 1, comprising a first polypeptide comprising an amino acid sequence of SEQ ID NO: 123, a second polypeptide comprising an amino acid sequence of SEQ ID NO: 122, a third polypeptide comprising an amino acid sequence of SEQ ID NO: 124, and a fourth polypeptide comprising an amino acid sequence of SEQ ID NO: 125.
